SEQUENCE LISTING (12 sequences)

SEQ ID NO: 1 - RNA, 21 nt, synthetic construct
  source 1..21: mol_type = other RNA
  modified_base 1: mod_base = OTHER; note = Carbocyclic 2'-O-methyl modified nucleotide e-vinyl phosphonate with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgcctaatgagccacactttt

SEQ ID NO: 2 - RNA, 21 nt, synthetic construct
  source 1..21: mol_type = other RNA
  modified_base 1: mod_base = OTHER; note = Carbocyclic 2'-O-methyl modified nucleotide e-vinyl phosphonate with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide with phosphorothioate linkage
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = Adenosine unlocked nucleic acid
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-O-methyl modified nucleotide with phosphorothioate linkage
  modified_base 21: mod_base = OTHER; note = 2'-O-methyl modified nucleotide
ttgcgaaacaattcgatatga

SEQ ID NO: 3 - RNA, 21 nt, synthetic construct
  source 1..21: mol_type = other RNA
  modified_base 1: mod_base = OTHER; note = R,S-dimethylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgcctaatgagccacactttt

SEQ ID NO: 4 - RNA, 21 nt, synthetic construct
  source 1..21: mol_type = other RNA
  modified_base 1: mod_base = OTHER; note = S,R-dimethylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgcctaatgagccacactttt

SEQ ID NO: 5 - RNA, 21 nt, synthetic construct
  source 1..21: mol_type = other RNA
  modified_base 1: mod_base = OTHER; note = R,R-dimethylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgcctaatgagccacactttt

SEQ ID NO: 6 - RNA, 21 nt, synthetic construct
  source 1..21: mol_type = other RNA
  modified_base 1: mod_base = OTHER; note = S,S-dimethylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgcctaatgagccacactttt

SEQ ID NO: 7 - RNA, 21 nt, synthetic construct
  source 1..21: mol_type = other RNA
  modified_base 1: mod_base = OTHER; note = cis-cyclobutylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide with phosphorothioate linkage
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = Adenosine unlocked nucleic acid
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-O-methyl modified nucleotide with phosphorothioate linkage
  modified_base 21: mod_base = OTHER; note = 2'-O-methyl modified nucleotide
ttgcgaaacaattcgatatga

SEQ ID NO: 8 - RNA, 21 nt, synthetic construct
  source 1..21: mol_type = other RNA
  modified_base 1: mod_base = OTHER; note = cis-cyclobutylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgcctaatgagccacactttt

SEQ ID NO: 9 - RNA, 21 nt, synthetic construct
  source 1..21: mol_type = other RNA
  modified_base 1: mod_base = OTHER; note = trans-cyclobutylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide with phosphorothioate linkage
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = Adenosine unlocked nucleic acid
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-O-methyl modified nucleotide with phosphorothioate linkage
  modified_base 21: mod_base = OTHER; note = 2'-O-methyl modified nucleotide
ttgcgaaacaattcgatatga

SEQ ID NO: 10 - RNA, 21 nt, synthetic construct
  source 1..21: mol_type = other RNA
  modified_base 1: mod_base = OTHER; note = trans-cyclobutylphosphonate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide
tgcctaatgagccacactttt

SEQ ID NO: 11 - RNA, 23 nt, synthetic construct
  source 1..23: mol_type = other RNA
  modified_base 1: mod_base = OTHER; note = Inverted abasic position with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-[15-(adamantly-1)pentadecyl]cytidine
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-fluoro modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with phosphorothioate linkage
  modified_base 22: mod_base = OTHER; note = 2'-O-methyl modified nucleotide with phosphorothioate linkage
  modified_base 23: mod_base = OTHER; note = Inverted abasic position
ntcatatcgaattgtttcgcaan

SEQ ID NO: 12 - RNA, 21 nt, synthetic construct
  source 1..21: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-fluoro modified nucleotide
  modified_base 4: mod_base = OTHER; note = 2'-O-methyl modified nucleotide
  modified_base 5: mod_base = OTHER; note = 2'-fluoro modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-fluoro modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-O-methyl modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-fluoro modified nucleotide
  modified_base 18: mod_base = OTHER; note = 2'-O-methyl modified nucleotide
  modified_base 19: mod_base = OTHER; note = 2'-fluoro modified nucleotide with phosphorothioate linkage
  modified_base 20: mod_base = OTHER; note = 2'-O-methyl modified nucleotide with phosphorothioate linkage
  modified_base 21: mod_base = OTHER; note = 2'-O-methyl modified nucleotide triethylene glycol cholesterol
aagtgtggctcattaggcatt

(19) United States
(12) Patent Application Publication
(10) Pub. No.: US 2026/0366019 A1
(43) Pub. Date: Nov. 19, 2026

(54) NOVEL STABILIZED PHOSPHATES

(71) Applicant: Janssen Biotech, Inc., Horsham, PA (US)

(72) Inventors: Marija Prhavc, Encinitas, CA (US); Yi Jin, Roseville, CA (US); Mahesh Ramaseshan, Fremont, CA (US); Renee Desjarlais, Spring House, PA (US); Ferdinand H. Lutter, Beerse (BE)

(21) Appl. No.: 19/113,445

(22) PCT Filed: Sep. 21, 2023
(86) PCT No.: PCT/IB2023/059349

§ 371 Date: Mar. 20, 2025

Related U.S. Application Data
(60) Provisional application No. 63/376,579, filed on Sep. 21, 2022.

(51) Int. Cl. C12N 15/113

(52) U.S. Cl. CPC C12N 15/113; C12N 2310/14; C12N 2310/3125

(57) ABSTRACT

The present disclosure includes oligonucleotides and nucleic acid compositions having stabilized phosphate moieties. Formula (Ia) and Formula (Ib), wherein: X is selected from a 3- to 5-membered cycloalkyl, —CHCH—, a 3- to 5-membered heterocycle, and —CH³CH³—.

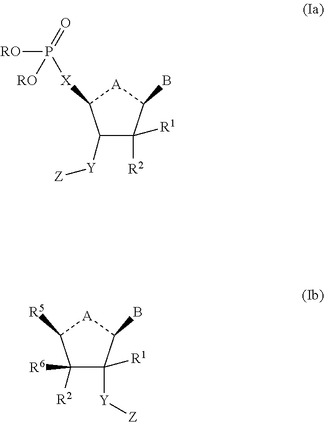

### CROSS REFERENCE TO RELATED APPLICATIONS

This application claims priority from U.S. Provisional Application No. 63/376,579 filed on Sep. 21, 2022, the entire contents of which are hereby incorporated by reference in their entirety.

### FIELD OF INVENTION

The present disclosure relates generally to the field of chemistry, molecular biology and genetic engineering, including oligonucleotides and nucleic acid moieties and molecules useful for stabilizing oligonucleotides, and the synthesis and use of the oligonucleotides and nucleic acid moieties and molecules.

### BACKGROUND

Efficient gene silencing by RNAi in vivo requires the recognition and binding of the 5′-phosphate of the guide strand of an siRNA by the Argonaute protein. It has been demonstrated that incorporation of a 5′-E-vinyl-phosphonate 5′-(E)-VP increases siRNA accumulation, extends duration of silencing, and protects siRNA from 5′-3′-exonuclease. ACS Chem. Biol. 2013, 8:7 1402-1406. Structural studies show that the 5′-binding site in the MID domain of human Argonaute-2 (hAgo-2) is able to adjust the key binding residues to compensate for the change introduced by the modified nucleotide. Nucleic Acids Res. 2017, 45:6 3528-3536.

There still exists a high unmet need for alternatives to 5′-(E)-VP.

### SUMMARY OF THE INVENTION

Accordingly, there is an unmet need for alternatives to 5′-(E)-VP. The invention satisfies this need by providing novel alternatives for 5′-(E)-VP.

In a general aspect, the present disclosure relates compounds represented by formula (Ia) or (Ib):

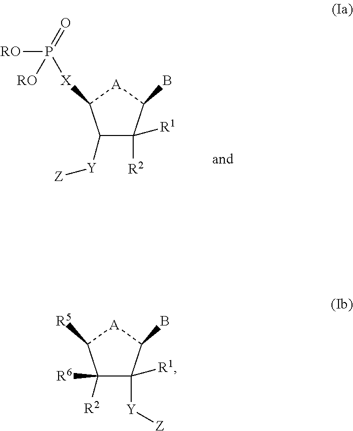

wherein: X is selected from a 3- to 5-membered cycloalkyl, —CHCH—, a 3- to 5-membered heterocycle, and —CHR³CHR³—; Y is selected from O or NR′; R′ is a counterion, H or a protecting group; Z is selected from H, a counterion, an activating group, and an oligonucleotide; A and the dashed lines connected are optional, and when present, A is selected from O, S, and CR⁴R⁴; B is a nucleobase; each R is independently selected from an oligonucleotide, a counterion, H and a protecting group, such as C₁-C₅ alkyl and POM or C₁-C₅ alkyl; each R¹ and R² is independently selected from H, F, OH, and an optionally substituted O-alkyl, or R¹ and R² form an optionally substituted oxetine; each R³ is independently selected from a C₁-C₃ alkyl; each R⁴ is independently selected from H, F and C₁-C₅ alkyl; one of R⁵ and R⁶ is XP(O)(OR)2 and the other is H; provided that when A is O, then X is not —CHCH— and when X is cyclopropyl, then at least one of A or Y is not O. In some embodiments, when A is present, and X is selected from a 3- to 5-membered cycloalkyl, —CHCH—, a 3- to 5-membered heterocycle, and —CHR³CHR³— then R¹ and R² are not each H.

In some embodiments, the compounds represented by the compound of formula (Ia) and (Ib) are represented by the following:

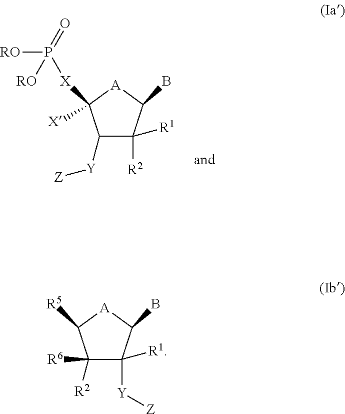

In some embodiments, the compound of formula (Ia) and (Ib) are represented by the following:

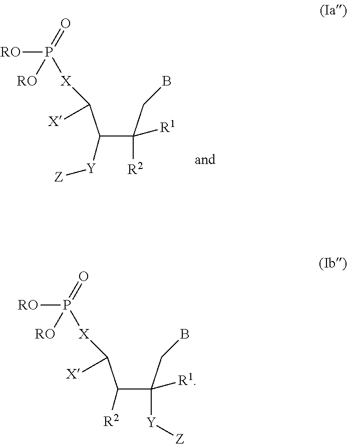

In some embodiments, the compound of formula (Ia) is represented by the following:

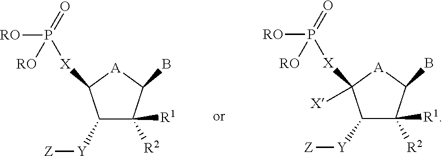

In some embodiments, X is a cyclobutyl. In some embodiments, the cyclobutyl is represented by the following:

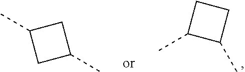

wherein the dashed lines represent connection points to adjacent atoms. In some embodiments, X is —CHR³CHR³—, and R³ is a methylene. In some embodiments, X is selected from the following:

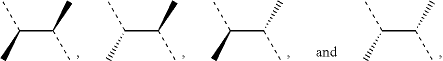

wherein the dashed lines represent connection points to adjacent atoms. In some embodiments, X is a 3- to 5-membered heterocycle selected from:

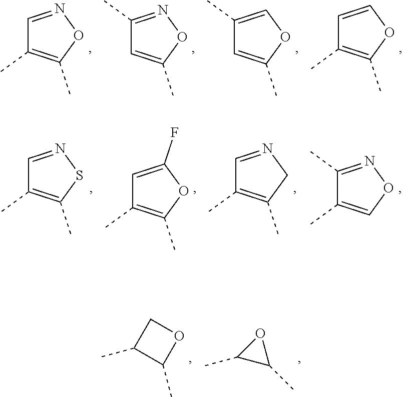

wherein the dashed lines represent connection points to adjacent atoms. In some embodiments, R² is an optionally substituted oxetine. In some embodiments, X is —CHCH—. In some embodiments, Y is O. In some embodiments, Z is an activating group. In some embodiments, the activating group is represented by:

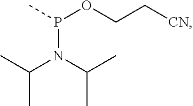

wherein the dashed line represents a connection point to the adjacent atom. In some embodiments, Z is an oligonucleotide. In some embodiments, the oligonucleotide is an antisense strand of RNA, preferably an antisense strand of siRNA. In some embodiments, the 5′-end of the oligonucleotide is connected to Y. In some embodiments, A is O. In some embodiments, A is CH₂ or CHF. In some embodiments, X is —CHCH—. In some embodiments, B is a uracil or thymine. In some embodiments, R¹ is H and R² is OMe, OEt, MOE, or F. In some embodiments, R is a protecting group, such as POM, Et, and Z is an activating group. In some embodiments, R is H and Z is an oligonucleotide. In other embodiments, R and Z are each oligonucleotide, e.g., the compound of the present disclosure is not at terminal end of an oligonucleotide. In some embodiments, R⁵ is XP(O)(OR)₂. In other embodiments, R⁶ is XP(O)(OR)₂.

In some embodiments, the compound is selected from:

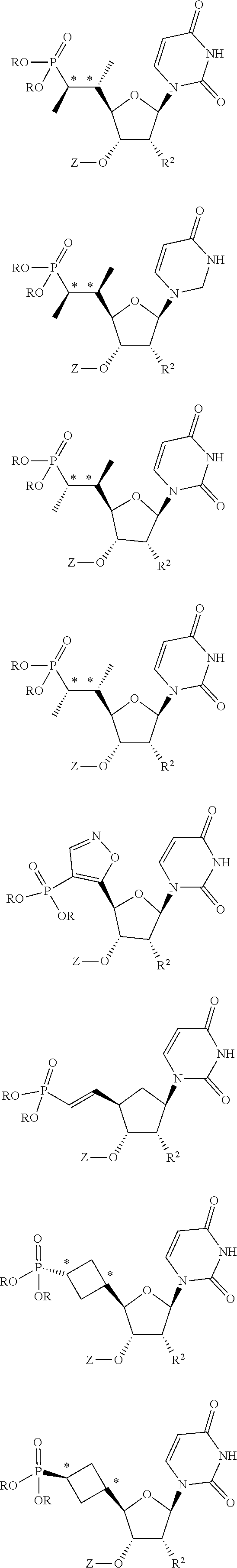

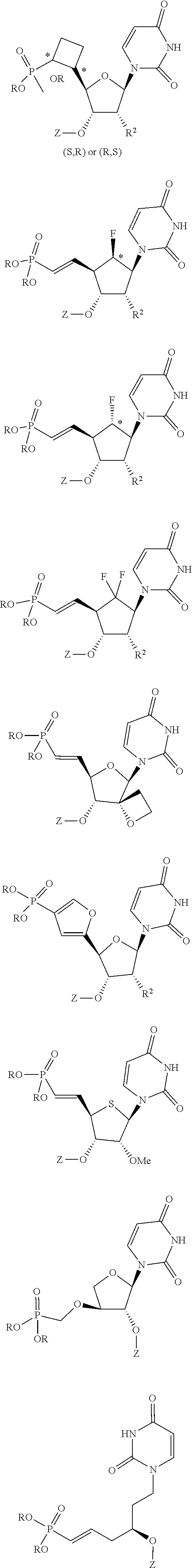

or a compound of the table wherein the nucleobase is thymine or cytosine. In some embodiments, R² is —OMe. In some embodiments, R is Et and Z is an activating group. In some embodiments, R is H or counterion and Z is an oligonucleotide.

Other aspects, features and advantages of the invention will be apparent from the following disclosure, including the detailed description of the invention and its preferred embodiments and the appended claims.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Unless otherwise stated, any numerical value, such as a % sequence identity or a % sequence identity range described herein, are to be understood as being modified in all instances by the term “about.” Thus, a numerical value typically includes ±10% of the recited value. For example, a dosage of 10 mg includes 9 mg to 11 mg. As used herein, the use of a numerical range expressly includes all possible subranges, all individual numerical values within that range, including integers within such ranges and fractions of the values unless the context clearly indicates otherwise.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of” or “consisting essentially of” to vary scopes of the disclosure.

As used herein “nucleobases” can include unmodified, natural or modified nucleobases. “Unmodified” or “natural” nucleobases include the purine bases adenine (A) and guanine (G), and the pyrimidine bases thymine (T), cytosine (C) and uracil (U). “Modified nucleobases” include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-am-oelhoxy)-H-pyrimido[5,4-b][1,4]benzoxazin-2(3H)-one), carbazole cytidine (2H-pyrimido[4,5-b]indol-2-one), pyridoindole cytidine (H-pyrido[3,2,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine, and 2-pyridone.

“Alkyl” refers to an optionally substituted saturated straight or branched chain hydrocarbon radical. Examples include without limitation methyl, ethyl, propyl, iso-propyl, butyl, iso-butyl, tert-butyl, n-pentyl and n-hexyl. “Cycloalkyl” refers to cyclic alkyl groups of from 3 to 10 carbon atoms having single or multiple cyclic rings including, by way of example, adamantyl, cyclopropyl, cyclobutyl, cyclopentyl, cyclohexyl, cyclooctyl and the like. In some embodiments, an optionally substituted alkyl is substituted with hone or more halogen, such as F.

As used herein “heterocycle” refers to a saturated, unsaturated, or aromatic ring system of 3 to 18 atoms that includes at least one N, O, S, or P. In some embodiments, the heterocycle includes one N, O, or S. In some embodiments, the heterocycle includes two N, O, or S.

As used herein, “protecting group” refers to a chemical modification of a functional group to obtain chemoselectivity in a subsequent chemical reaction. Examples of protecting groups include those disclosed, e.g., in Greene Wuts, Protective Groups in Organic Synthesis.

The term “activating group” includes a moiety that increases the capability of the group to form a covalent bond with another molecule. Activating groups include phosphite-triester, phosphotriester, H-phosphonate, or preferably phosphoramidite group on at least one of the oxygen atoms of the sugar moiety. Preferably, the activating group is on the C-3′ oxygen or C-5′ oxygen of the nucleic acid monomer. Typically, the activating group is on the C-3′ oxygen of the nucleic acid monomer, for synthesizing probes in the 3′→5′ direction, with the oligonucleotide attached to the support via the 3′-end. The activating group is on the C-5′ oxygen of the nucleic acid monomer, for synthesizing probes in the 5′→3′ (“reverse”) direction, with the oligonucleotide attached to the support via the 5′-end.

In an attempt to help the reader of the application, the description has been separated in various paragraphs or sections or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

### Compounds of the Present Disclosure

In a general aspect, the present disclosure relates compound represented by formula (Ia) or (Ib):

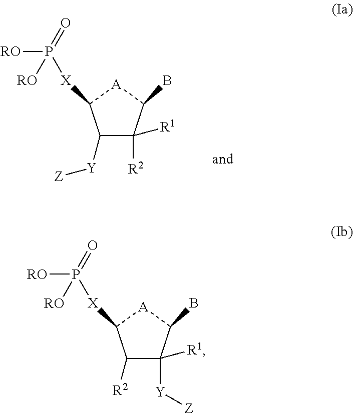

wherein: X is selected from a 3- to 5-membered cycloalkyl, —CHCH—, a 3- to 5-membered heterocycle, and —CHR³CHR³—; Y is selected from O or NR′; R′ is a counterion, H or a protecting group; Z is selected from H, a counterion, an activating group, and an oligonucleotide; A and the dashed lines connected are optional, and when present, A is selected from O, S, and CR⁴R⁴; B is a nucleobase; each R is independently selected from an oligonucleotide, a counterion, H and a protecting group, such as C₁-C₅ alkyl and POM or C₁-C₅ alkyl; each R¹ and R² is independently selected from H, F, OH, and an optionally substituted O-alkyl, or R¹ and R² form an optionally substituted oxetine; each R³ is independently selected from a C₁-C₃ alkyl; each R⁴ is independently selected from H, F and C₁-C₅ alkyl; provided that when A is O, then X is not —CHCH— and when X is cyclopropyl, then at least one of A or Y is not O. In some embodiments, when A is present, and X is selected from a 3- to 5-membered cycloalkyl, —CHCH—, a 3- to 5-membered heterocycle, and —CHR³CHR³— then R¹ and R² are not each H.

In some embodiments, the compound of formula (Ia) and (Ib) are represented by the following:

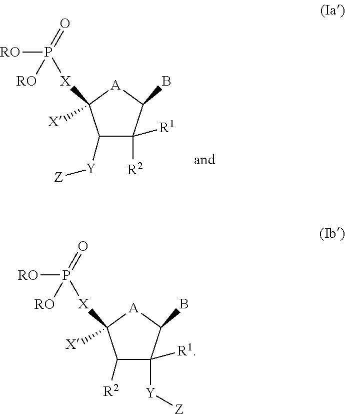

In some embodiments, the compound of formula (Ia) and (Ib) are represented by the following:

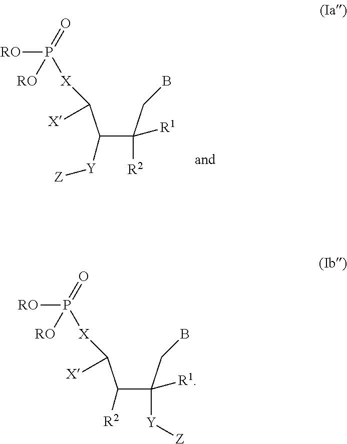

In some embodiments, the compound of formula (Ia) is represented by the following:

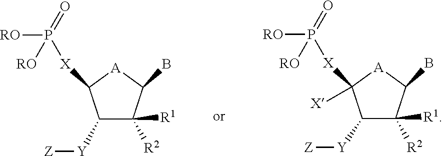

In some embodiments, X is a cyclobutyl. In some embodiments, the cyclobutyl is represented by the following:

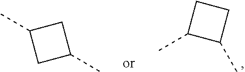

wherein the dashed lines represent connection points to adjacent atoms. In some embodiments, X is —CHR³CHR³—, and R³ is a methylene. In some embodiments, X is selected from the following:

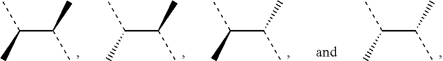

wherein the dashed lines represent connection points to adjacent atoms. In some embodiments, X is a 3- to 5-membered heterocycle has a structure:

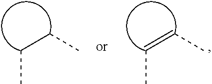

where the curved line includes one or more heteroatom (e.g., N, O, or S). In some embodiments, X is a 3- to 5-membered heterocycle selected from:

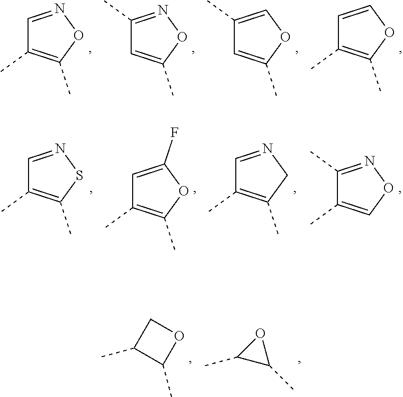

wherein the dashed lines represent connection points to adjacent atoms. In some embodiments, Y is O. In some embodiments, Y is O and X is not cyclopropyl.

In some embodiments, Z is an activating group. In some embodiments, Z is a phosphoramidite or functionally similar moiety. In some embodiments, the activating group is represented by:

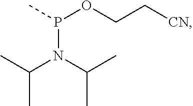

wherein the dashed line represents A connection point to the adjacent atom.

In some embodiments, Z is an oligonucleotide. In some embodiments, the oligonucleotide is an antisense strand of RNA, preferably an antisense strand of siRNA. In some embodiments, the 5′-end of the oligonucleotide is connected to Y.

In some embodiments, A is O. In some embodiments, A is O and X is not cyclopropyl. In some embodiments, A is CH₂ or CHF.

In some embodiments, R¹ and R² are independently selected from H, F, OH, and an optionally substituted O-alkyl, provided that when X is selected from a 3- to 5-membered cycloalkyl, —CHCH—, a 3- to 5-membered heterocycle, and —CHR³CHR³— then R¹ and R² are not each H, or R¹ and R² form an optionally substituted oxetine. In some embodiments, the substituted O-alkyl includes —OMe, —OEt, —CH₂CH₂OCH₃ (or MOE), —CF₂CH₂OCH₃, —CH₂CF₂OCH₃, —CH₂CH₂OCF₃, —CF₂CF₂OCH₃, —CH₂CF₂OCF₃, —CF₂CH₂OCF₃, —CF₂CF₂OCF₃, —CHFCH₂OCH₃, —CHFCHFOCH₃, —CHFCH₂OCFH₂, —CHFCH₂OCHF₂ and —CH₂CHFOCH₃, and the like.

In some embodiments, B is a uracil. In some embodiments, R¹ is H and R² is OMe, OEt, MOE, or F. In some embodiments, R is a protecting group, such as POM, C₁-C₅ alkyl (e.g., Et), and Z is an activating group. In some embodiments, R is H and Z is an oligonucleotide.

In some embodiments, the compound is selected from:

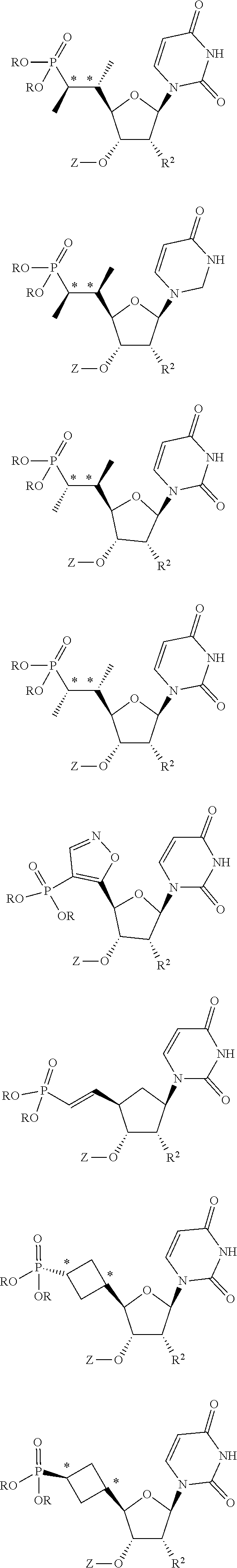

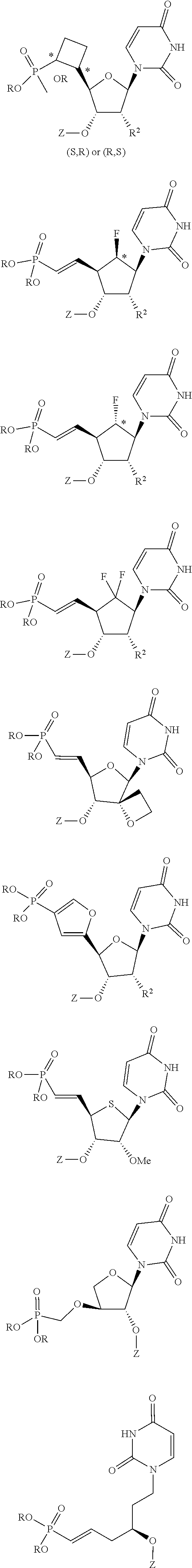

or a compound of the table wherein the nucleobase is thymine or cytosine.

In some embodiments, R² is selected from H, F, OH, and an optionally substituted O-alkyl, such as OMe, OEt, MOE, etc. In some embodiments, R² is —OMe. In some embodiments, each R is independently selected from an oligonucleotide, a counterion, H and a protecting group, such as POM or C₁-C₅ alkyl. In some embodiments, R is a protecting group, such as POM or C₁-C₅ alkyl (e.g., Et) and Z is an activating group, such as a phosphoramidite or functionally similar moiety. In some embodiments, the activating group is represented by:

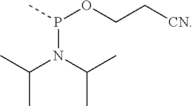

In some embodiments, R is H and Z is an oligonucleotide.

### EXAMPLES

The following examples are offered to illustrate but not to limit the invention(s) of the present disclosure. One of skill in the art will recognize that the following procedures may be modified using methods known to one of ordinary skill in the art.

### GLOSSARY

The following abbreviations are used herein:
m=2′-O-Methyl modified
f=2′-fluoro modified
VP=vinyl phosphonate
(invabasic)=inverse abasic
ps=phosphorothioate
TEG-Chol=Cholesterol-triethylene glycol
ecc); cc=carbocyclic; e refers to the geometric isomer of vinyl phosphonate
VP(ecc)mU=carbocyclic mU e-vinyl phosphonate
DMP(RS)=R,S-dimethylphosphonate
DMP(SR)=S,R-dimethylphosphonate
DMP(RR)=R,R-dimethylphosphonate
DMP(SS)=S,S-dimethylphosphonate
P(ccb)=cis-cyclobutylphosphonate
P(tcb)=trans-cyclobutylphosphonate
(UNAA)=should be (UNA-A)
(UNA-A)=adenosine unlocked nucleic acid
(J15AdaC)=should be J2-C15AdaC=2′-O-[15-(adamantly-1)pentadecyl]cytidine

### Example 1: Synthesis of Dimethyl Phosphonates

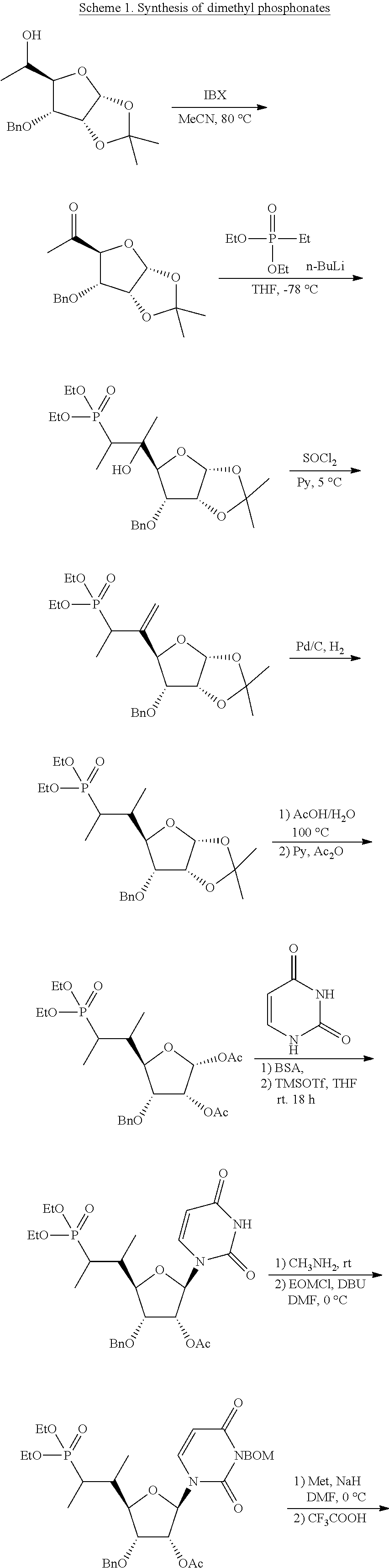

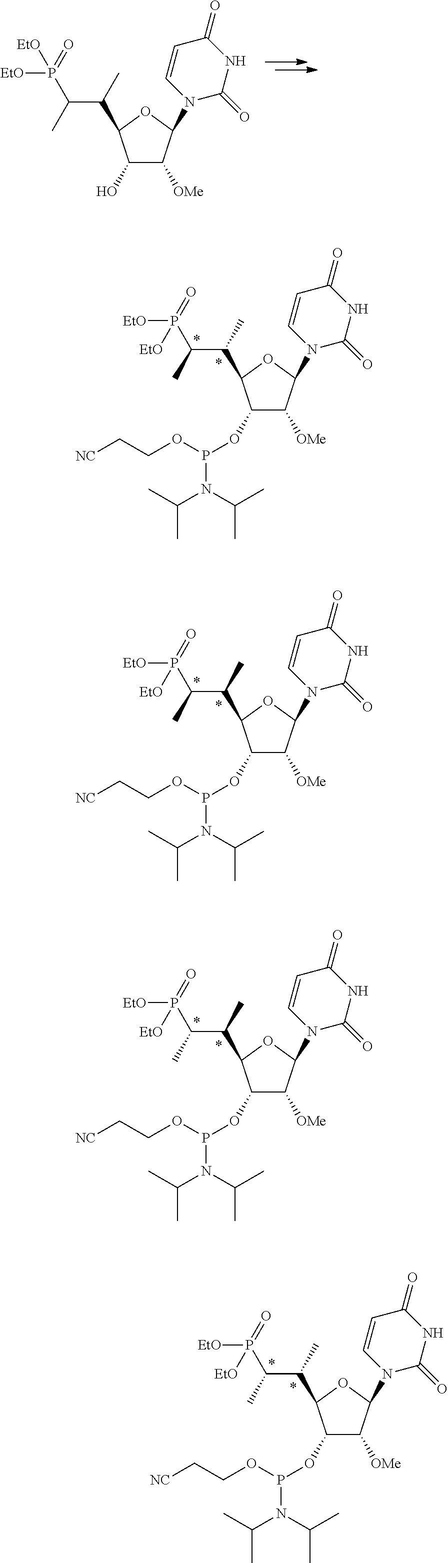 [SMILES: CCOP(=O)(OCC)C(C)C(C)[C@H]1O[C@@H](n2ccc(=O)[nH]c2=O)[C@H](OC)[C@@H]1O ; CCOP(=O)(OCC)[C@H](C)[C@H](C)[C@H]1O[C@@H](n2ccc(=O)[nH]c2=O)[C@H](OC)[C@@H]1OP(OCCC#N)N(C(C)C)C(C)C ; CCOP(=O)(OCC)[C@H](C)[C@@H](C)[C@H]1O[C@@H](n2ccc(=O)[nH]c2=O)[C@H](OC)[C@@H]1OP(OCCC#N)N(C(C)C)C(C)C ; CCOP(=O)(OCC)[C@@H](C)[C@@H](C)[C@H]1O[C@@H](n2ccc(=O)[nH]c2=O)[C@H](OC)[C@@H]1OP(OCCC#N)N(C(C)C)C(C)C ; CCOP(=O)(OCC)[C@@H](C)[C@H](C)[C@H]1O[C@@H](n2ccc(=O)[nH]c2=O)[C@H](OC)[C@@H]1OP(OCCC#N)N(C(C)C)C(C)C]

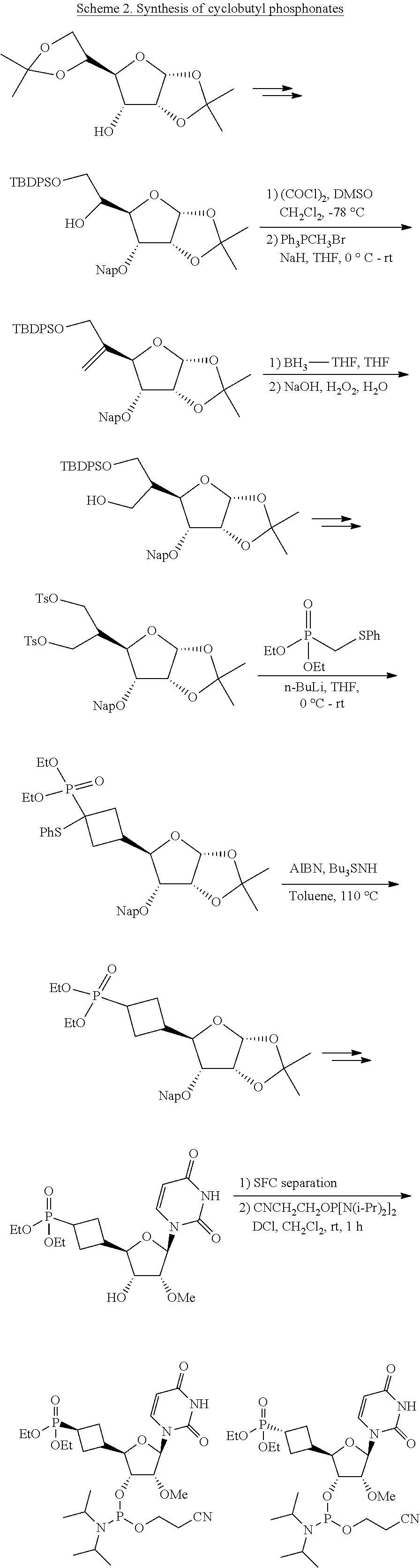

### Example 2: Synthesis of Phosphonates

### Example 2-1

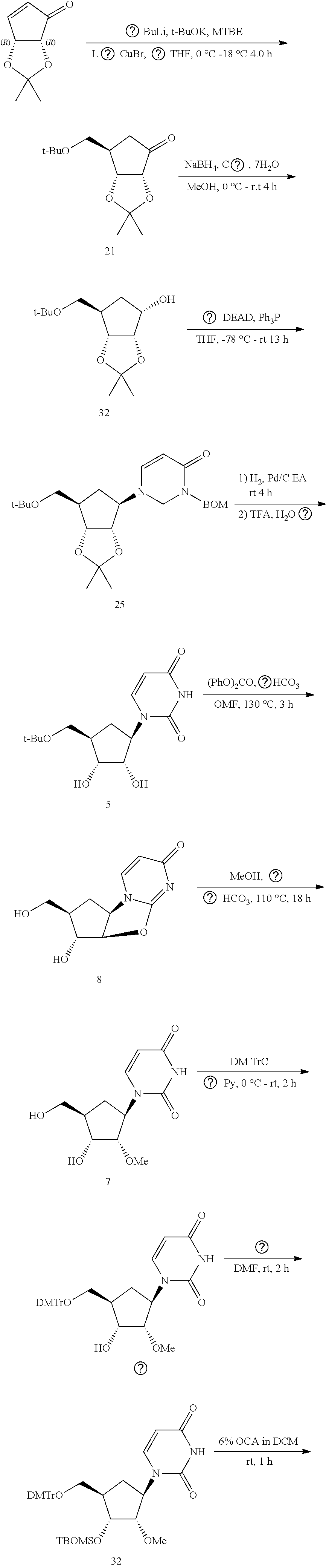

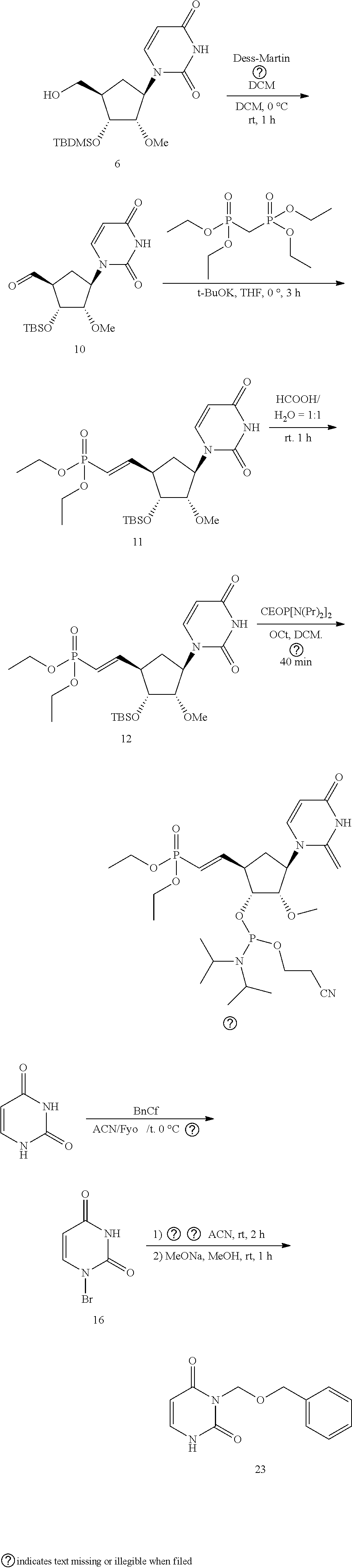

To a soltion of potasium tert-butoxide (65.5 g, 538.8 mmol, 3.0 equiv) in tert-Butyl methyl ether (1300 mL) with an inert atmosphere of argon was added sec-butylithium (450 mL, 583.8 mmol, 3.0 equiv) dropwise was stirring at −78 °C for 2.5 hours. The resulting solution was added Lithium romtoide (101.5 g, 1167.5 mmol, 6.0 equiv) in tetrahydrofuran (1000 mL) dropwise with stirring at −78′°C. The resulting solution was stirring for 30 min at −15 °C. The resulting solution was cooled to −78 °C. The resulting solution was added copper(I) bromide-dimethyl sulfide (60 g, 291.9 mmol, 1.5 equiv) in 6-O-(triisopropylsilyl)-d-galactal cyclic carbonate (420 mL) at −78 °C. The resulting solution was stirred at −78 °C for 1 hour. The resulting solution was added (3aR,6aR)-2,2-dimethyl-3a,6a-dihydro-4H-cyclopenta[d][1,3]dioxol-4-one (30 g, 194.6 mmol, 1.0 equiv) in tetrahydrofuran (300 mL) at −78 °C. The resulting solution was stirring at −30 °C for 30 min. The reaction mixture was diluted with 2000 mL of tert-butyl methyl ether. The reaction mixture was quenched by 100 mL of the addition of acetic acid and methanol (v/v=1:1). The organic layer was washed by 3×3000 mL of mixed aqueous ammonium chloride and ammonia solution (PH=9). The organic phase was dried over anhydrous sodium sulfate, filtered and concentrated under reduced pressure. The crude product was applied onto a silica gel column with petroleum ether/ethyl acetate (100:1-50:1). 35 g (74% yield) of 21 was obtained as a white solid. MS m/z [M+NH₄⁺]⁺ (ESI): 260.30. ¹H NMR (300 MHz, DMSO-d6) δ 4.60 (d, J=5.4 Hz, 1H), 4.11 (d, J=5.4, 1.1 Hz, 1H), 3.49 (dd, J=8.7, 2.7 Hz, 1H), 3.35-3.29 (m, 1H), 2.61 (dd, J=17.7, 8.9 Hz, 1H), 2.49-2.42 (m, 1H), 1.95-1.84 (m, 1H), 1.33 (s, 3H), 1.27 (s, 3H), 1.06 (s, 9H).

To a solution of 21 (20 g, 123.96 mmol, 1.0 equiv), cerium(III) chloride heptahydrate (46.172 g, 123.96 mmol, 1.0 equiv) in methanol (180 mL) with an inert atmosphere of argon was added borohydride (7.034 g, 185.94 mmol, 1.5 equiv) slowly at 0 °C. The resulting solution was stirring for 4 h at room temperature. The reaction mixture was quenched by the addition of water. The resulting solution was extracted with 300 mL of Ethyl acetate. The organic layers combined and was washed with 300 mL of brine. The organic layer was dried over anhydrous sodium sulfate, filtered and concentrated under vacuum. The crude product was applied onto a silica gel column with Petroleum ether/ethyl acetate (50:1-20:1). 18 g (89% yield) of 21 was obtained as a light-yellow liquid.

To a solution of pyrimidine-2,4(1H,3H)-dione (100 g, 892.85 mmol, 1.0 equiv) in 840 mL of acetonitrile and 160 mL of pyridine with an inert atmosphere of argon was added benzoyl chloride (114.05 mL, 982.14 mmol, 1.1 equiv) dropwise at 0 °C. The resulting solution was stirred at room temperature for 5 hours. The reaction mixture was quenched with 200 mL of 1 N Hydrochloric acid, and diluted with 500 mL of dichloromethane, and washed with 3×200 mL of saturated aqueous sodium chloride. The organic layers combined and dried over anhydrous sodium sulfate and concentrated under vacuum. The residue was applied onto a silica gel column with Petroleum ether/ethyl acetate (10:1-1:1). 60 g (31% yield) 19 was obtained as a white solid.

To a solution of 19 (60 g, 277.78 mmol, 1.0 equiv), 1,8-Diazabicyclo[5.4.0]undec-7-ene (48 mL) in acetonitrile (600 mL) with an inert atmosphere of argon was added benzylchloromethyl ether (52 g, 333.33 mmol, 1.2 equiv). The resulting solution was stirred at room temperature for 2 hours. The reaction was then concentrated under vacuum. The residue was extracted with ethyl acetate (250 ml) and washed with 1×200 mL of water, 1×200 mL of saturated aqueous sodium chloride. The organic layers were dried over anhydrous sodium sulfate and concentrated under vacuum. The residue was dissolved in 600 mL of sodium methoxide (2M sodium methoxide in methanol) and stirred for 1 h at room temperature with an inert atmosphere of argon. The reaction mixture was then evaporated to dryness. The residue was applied onto a silica gel column with Petroleum ether/ethyl acetate (2:1-1:3). 55 g (85% yield) of 20 was obtained as a white solid. MS m/z [M−H]⁻ (ESI): 231.00.

To a solution of 22 (28.5 g, 116.8 mmol, 1.0 equiv), 20 (40.6 g, 175.2 mmol, 1.5 equiv), triphenylphosphine (76.6 g, 292.0 mmol, 2.5 equiv) in tetrahydrofuran (1000 mL) with an inert atmosphere of argon was added dropwise diethyl azodicarboxylate (50.85 g, 292.0 mmol, 2.5 equiv) slowly at −78 °C. The resulting solution was stirring at −78 °C for 1 hour and was then slowly warmed up to room temperature, stirred for 16 h. The resulting mixture was concentrated under vacuum. The residue was purified by Flash-Prep-HPLC with the following conditions: Column, C18 silica gel; mobile phase, water and acetonitrile (30% acetonitrile up to 100% in 20 min), UV 254 nm. The fractions were diluted with dichloromethane, dried over anhydrous sodium sulfate and concentrated under reduced pressure. 36.5 g (68% yield) of 23 was obtained as a yellow oil. MS m/z [M+H]⁺ (ESI): 459.30. ¹H NMR (300 MHz, Chloroform-d) δ 7.32 (t, J=3.3 Hz, 5H), 7.20 (d, J=7.9 Hz, 1H), 5.71 (d, J=7.8 Hz, 1H), 5.39-5.15 (m, 3H), 5.03 (dt, J=6.8, 3.5 Hz, 1H), 4.61 (d, J=3.2 Hz, 2H), 4.52 (t, J=6.6 Hz, 1H), 3.62 (dd, J=8.7, 4.6 Hz, 1H), 3.37-3.25 (m, J=8.3, 5.9 Hz, 1H), 2.33-2.03 (m, 3H), 1.52 (s, 3H), 1.28 (s, 3H), 1.17 (s, 9H).

To a solution of 23 (35 g, 76.4 mmol, 1.0 equiv) in ethyl acetate (350 mL) with an inert atmosphere of hydrogen was added 10% Pd/C (wₜ/wₜ=10%, 3.5 g), trifluoroacetic acid (35 mL) in order at room temperature. The resulting solution was stirred at room temperature for 4 hours. The solution was filtered and concentrated under reduced pressure. The residue was dissolved in 350 mL of trifluoroacetic acid/water=1:1 at room temperature. The resulting solution was stirred for 2 hours with an inert atmosphere of argon at 50 °C. The solution was concentrated under reduced pressure. The residue was dissolved in the solution of 1N ammonium hydroxide in methanol (200 mL). The resulting mixture was concentrated under vacuum. The residue was purified by Flash-Prep-HPLC with the following conditions: Column, C18 silica gel; mobile phase, water and acetonitrile (0% acetonitrile up to 30% in 15 min), UV 254 nm. The fractions were concentrated under reduced pressure. 15.7 g (85% yield) of −5 was obtained as a light yellow oil. MS m/z [M+Na]⁺ (ESI): 265.00.

To a solution of 5 (17 g, 70.24 mmol, 1.0 equiv) in N,N-dimethylformamide (170 mL) with an inert atmosphere of argon was added diphenyl carbonate (22.57 g, 105.37 mmol, 1.5 equiv), sodium bicarbonate (590.38 mg, 7.02 mmol, 0.1 equiv) in order. The reaction mixture was heated at 150 °C for 3 hours until the solution had turned to a dark red. The reaction mixture was cooled to room temperature and poured slowly into 3000 mL of diethyl ether with stirring. The deposit was collected by filtered, redissolved in methanol, decolorized with charcoal, and filtered. The filtrate was concentrated under vacuum. 10.3 g (73% yield) of 6 was obtained as a yellow solid. MS m/z [M−H]⁻ (ESI): 223.00.

To a solution of 6 (14.7 g, 65.62 mmol, 1.0 equiv) in methanol (150 mL) was added trimethyl borate (13.6 g, 131.25 mmol, 2.0 equiv), trimethoxymethane (6.96 g, 65.6 mmol, 1.0 equiv), sodium bicarbonate (1.1 g, 13.1 mmol, 0.2 equiv) in order. The reaction mixture was at 150 °C stirred for 16 hours in sealed tube. The reaction mixture was cooled to room temperature and concentrated under vacuum. The residue was purified by Flash-Prep-HPLC with the following conditions: Column, C18 silica gel; mobile phase, water and acetonitrile (0% acetonitrile up to 40% in 20 min), UV 254 nm. The fractions were concentrated under reduced pressure. 7.7 g (45% yield) of 7 was obtained as a light yellow oil. MS m/z [M−H]⁻ (ESI): 255.00.

To a solution of 7 (5 g, 19.53 mmol, 1.0 equiv) in pyridine (50 mL) with an inert atmosphere of argon was added 4,4′-dimethoxytrityl chloride (6.94 g, 20.5 mmol, 1.05 equiv) at 0 °C. The resulting solution was stirred at room temperature for 2 hours. The reaction mixture was quenched by 10 mL of methanol, diluted with 200 mL of dichloromethane, washed with 2×150 mL of saturated aqueous sodium bicarbonate and 2×150 mL of saturated aqueous sodium chloride respectively. The organic layer was dried over anhydrous sodium sulfate, filtered and concentrated under reduced pressure. The residue was purified by Flash-Prep-HPLC with the following conditions: Column, C18 silica gel; mobile phase, water and acetonitrile (30% acetonitrile up to 100% in 20 min), UV 254 nm. The fractions were concentrated under reduced pressure. 8.4 g (77% yield) of 31 was obtained as a light yellow solid. MS m/z [M−H]⁻ (ESI): 557.10.1H NMR (300 MHz, DMSO-d6) δ 11.13 (s, 1H), 7.45-7.35 (m, 3H), 7.35-7.18 (m, 7H), 6.93-6.84 (m, 4H), 5.57 (d, J=7.5 Hz, 1H), 5.10-4.97 (m, 1H), 4.37 (d, J=7.3 Hz, 1H), 4.14-3.99 (m, 1H), 3.90 (t, 1H), 3.74 (s, 6H), 2.89 (t, J=7.9 Hz, 1H), 3.23-3.15 (m, 3H), 1.96-1.69 (m, 2H), 1.35-1.08 (m, 1H), 0.83 (t, J=7.3 Hz, 1H).

To a solution of 31 (8.4 g, 15.05 mmol, 1.0 equiv) in N,N-Dimethylformamide (85 mL) with an inert atmosphere of argon was added imidazole (2.55 g, 37.59 mmol, 2.5 equiv) and tert-butyldimethylsilyl chloride (5.66 g, 37.59 mmol, 2.5 equiv) in order at room temperature. The resulting solution was stirred at room temperature for 2 hours. The reaction mixture was diluted with 500 mL of dichloromethane, washed with 2×200 mL of saturated aqueous sodium bicarbonate and 1×200 mL of saturated aqueous sodium chloride respectively. The organic phase was dried over anhydrous sodium sulfate, filtered and concentrated under reduced pressure. The residue was purified by Flash-Prep-HPLC with the following conditions: Column, C18 silica gel; mobile phase, water and acetonitrile (30% acetonitrile up to 100% in 20 min), UV 254 nm. The fractions were concentrated under reduced pressure. 8.6 g (81% yield) of 32 was obtained as a light yellow oil. MS m/z [M−H]⁻ (ESI): 671.30.

To a solution of 32 (10.0 g, 14.88 mmol, 1.0 equiv) was dissolved in 100 mL of 6% dichloroacetic acid in dichloromethane with an inert atmosphere of argon was added triethylsilane(5.2 g, 44.64 mmol, 3.0 eq). The resulting solution was stirred at room temperature for 1 hour. The reaction mixture was diluted with 200 mL of dichloromethane, washed with 2×200 mL of saturated aqueous sodium bicarbonate, 1×100 mL of water and 1×200 mL of saturated aqueous sodium chloride respectively. The organic phase was dried over anhydrous sodium sulfate, filtered and concentrated under reduced pressure. The residue was purified by Flash-Prep-HPLC with the following conditions: Column, C18 silica gel; mobile phase, water and acetonitrile (30% acetonitrile up to 100% in 20 min), UV 254 nm. The fractions were concentrated under reduced pressure. 4.2 g (76%) of 31 was obtained as a white solid. MS m/z [M−H]⁻ (ESI): 369.15.1H NMR (300 MHz, DMSO-d6) δ 11.09 (d, J=5.8 Hz, 1H), 7.42 (dd, J=7.6, 5.8 Hz, 1H), 5.57 (dd, J=7.6, 1.5 Hz, 1H), 5.05 (td, J=9.7, 5.5 Hz, 1H), 4.52 (t, J=5.2 Hz, 1H), 4.26 (dd, J=6.7, 5.4 Hz, 1H), 4.02 (t, J=5.4 Hz, 1H), 3.49-3.59 (m, J=10.6, 5.3 Hz, 1H), 3.33-3.27 (m, 1H), 3.17 (s, 3H), 1.90-2.03 (m, J=7.5 Hz, 1H), 1.86-1.56 (m, 2H), 0.88 (s, 9H), 0.06 (d, 6H).

To a solution of 9 (2.5 g, 6.74 mmol, 1.0 equiv) in 25 mL of dichloromethane with an inert atmosphere of argon was added Dess-Martin periodinane (4.0 g, 10.12 mmol, 1.5 equiv) at 0 °C. The resulting solution was stirred at room temperature for 1 hour. The reaction mixture was diluted with 300 mL of dichloromethane, washed with 1×200 mL saturated aqueous sodium thiosulfate, 1×200 mL of saturated aqueous sodium bicarbonate, 2×100 mL of water and 1×200 mL of saturated aqueous sodium chloride respectively. The organic phase was dried over anhydrous sodium sulfate, filtered and concentrated under reduced pressure. 2.1 g (84%) of 10 was obtained as a white solid. MS m/z [M−H]⁻ (ESI): 367.05. It was used in next step without further purification.

To a solution of tetraethyl methylenebis(phosphonate) (2.13 g, 7.41 mmol, 1.3 equiv) in anhydrous tetrahydrofuran (20 mL) was added potassium tert-butoxide (2.13 g, 5.70 mmol, 1.35 equiv) at 0 °C. The reaction mixture was stirred at 0 °C for 10 min, then warmed up to room temperature for 30 min. Then a solution of 10 (2.10 g, 5.70 mmol, 1.0 equiv) in 20 mL of anhydrous tetrahydrofuran was added dropwise at 0 °C. The resulting solution was stirred at 0 °C for 3 hours. The reaction mixture was diluted with 200 mL of dichloromethane, washed with 2×200 mL of saturated aqueous sodium bicarbonate. The organic phase was dried over anhydrous sodium sulfate, filtered and concentrated under reduced pressure. The residue was purified by Flash-Prep-HPLC with the following conditions: Column, C18 silica gel; mobile phase, water and acetonitrile (30% acetonitrile up to 100% in 20 min), UV 254 nm. The fraction was dilute with equal volume of dichloromethane. The organic phase layer was separated. The aqueous phase was extracted with 3×20 mL of dichloromethane. The organic phase was combined and dried over anhydrous sodium sulfate. The solid was filtered out. The filtrate was concentrated at 25 °C. 2 g (68%) of 12 was obtained as a light yellow solid. MS m/z [M+H]⁺ (ESI): 503.20.1H NMR (300 MHz, DMSO-d₆) δ 11.18 (s, 1H), 7.45 (d, J=7.6 Hz, 1H), 6.65-6.48 (m, J=21.9, 17.1, 7.6 Hz, 1H), 5.90-5.81 (m, 1H), 5.61 (d, J=7.5 Hz, 1H), 5.06-4.95 (m, 1H), 4.40 (dd, J=9.8, 5.8 Hz, 1H), 4.00-3.89 (m, J=8.3, 7.0, 1.3 Hz, 4H), 3.81 (dd, J=5.9, 2.6 Hz, 1H), 3.21 (s, 3H), 2.75 (s, 1H), 1.93-0.73 (m, 2H), 1.22 (t, J=7.0 Hz, 6H), 0.87 (s, 9H), 0.03 (s, 6H). ³¹P NMR (121 MHz, DMSO-d6) δ 17.62.

The 11 (2.00 g, 5.15 mmol, 1.0 equiv) was dissolved in 20 mL of formic acid and H₂O (v/v=1:1) with an inert atmosphere of argon. The mixture solution was stirred at room temperature for 1 hour. The resulting solution was concentrated under vacuum. The residue was purified by Flash-Prep-HPLC with the following conditions: Column, C18 silica gel; mobile phase, water and acetonitrile (15% acetonitrile up to 35% in 20 min), UV 254 nm. The fractions were concentrated under reduced pressure. 1.3 g (84%) of 12 was obtained as a white solid. MS m/z [M+H]⁺ (ESI): 389.25. ¹H NMR (300 MHz, DMSO-d6) δ 11.17 (d, 1H), 7.48-7.38 (m, 1H), 6.76-6.55 (m, 1H), 5.87-5.72 (m, 1H), 5.60 (d, 1H), 5.02 (td, 1H), 4.74 (d, 1H), 4.17-4.07 (m, 1H), 4.02-3.90 (m, 4H), 3.88-3.83 (m, 1H), 3.26 (s, 3H), 2.75-2.58 (m, 1H), 1.94-1.72 (m, 2H), 1.23 (td, 6H). ³¹P NMR (121 MHz, DMSO-d6) δ 18.08.

To a solution of 12 (1.7 g, 4.38 mmol, 1.0 equiv) in dichloromethane (170 mL) was added opylamino)phosphinooxy)propanenitrile (1.72 g, 5.69 mmol, 1.3 equiv),4,5-Dicyanoimidazole (569 mg, 4.88 mmol, 1.1 equiv) in order. The resulting solution was stirred at room temperature for 40 min. The resulting solution was diluted with 500 mL of dichloromethane, washed with 1×300 mL of sodium bicarbonate and 1×300 mL of brine respectively. The organic layer was dried over anhydrous sodium sulfate, filtered and concentrated under vacuum. The crude product was purified by Flash-Prep-HPLC with the following conditions (IntelFlash-1): Column, C18 silica gel; mobile phase, water and acetonitrile (30% acetonitrile up to 100% in 10 min and hold 100% for 5 min); Detector, UV 254 nm. The fraction was dilute with equal volume of dichloromethane. The organic phase layer was separated. The aqueous phase was extracted with 3×50 mL of dichloromethane. The organic phase was combined and dried over anhydrous sodium sulfate. The solid was filtered out. The filtrate was concentrated at 25 °C, obtained the product which was dried for 4 hours under reduced pressure at 25 °C. 1.85 g of 0 was obtained as a white solid. MS m/z [M−H]⁻ (ESI): 587.10. ¹H NMR (300 MHz, DMSO-d6) δ 11.14 (s, 1H), 7.42 (dd, J=7.6, 1.1 Hz, 1H), 6.67-6.49 (m, 1H), 5.95-5.69 (m, 1H), 5.59 (d, J=7.6 Hz, 1H), 5.10-4.98 (m, 1H), 4.53-4.29 (m, 1H), 4.02-3.83 (m, 5H), 3.80-3.66 (m, 2H), 3.63-3.47 (m, 2H), 3.22 (d, J=3.0 Hz, 3H), 2.85 (s, 1H), 2.79-2.71 (m, 2H), 1.96-1.76 (m, 2H), 1.24-1.17 (m, 6H), 1.15-1.11 (m, 12H). ³¹P NMR (121 MHz, DMSO) δ 147.53, 146.78, 17.69, 17.50.

### Example 2-2

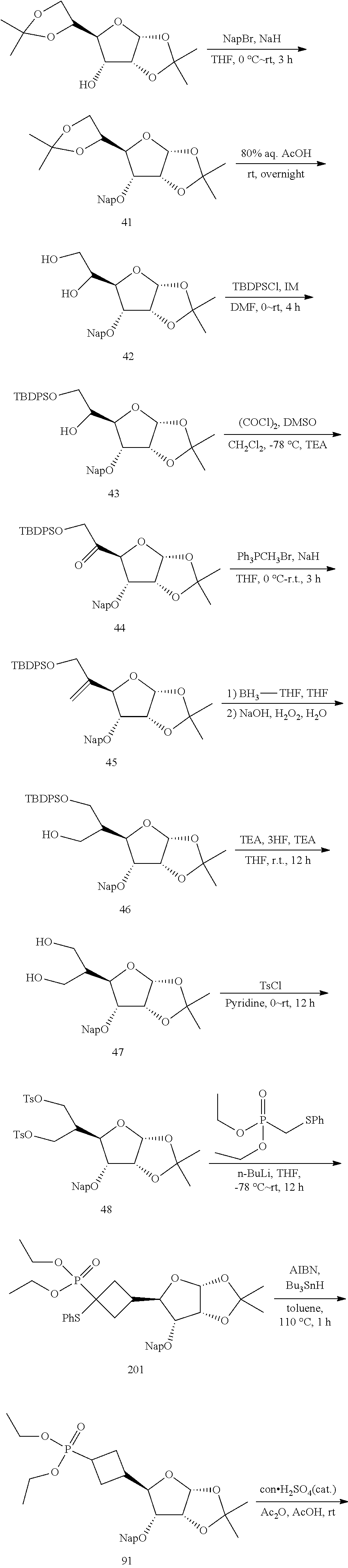

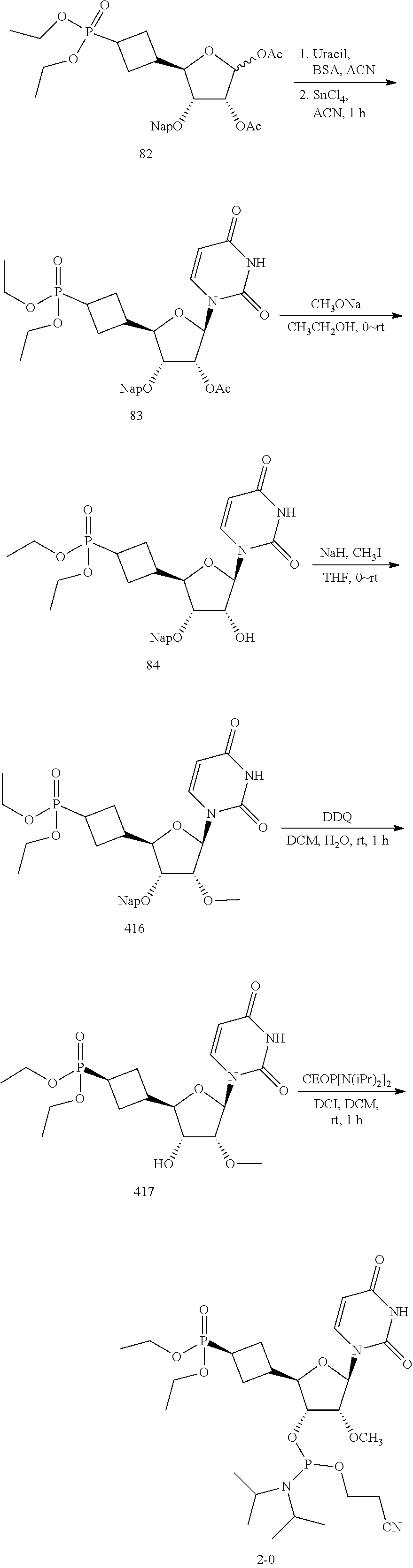

To a solution of (3aR,5S,6R,6aR)-5-((R)-2,2-dimethyl-1,3-dioxolan-4-yl)-2,2-dimethyltetrahydrofuro [2,3-d][1,3]dioxol-6-ol (400 g, 1.54 mol, 1.0 equiv) in 4 L of tetrahydrofuran with an inert atmosphere of nitrogen was added sodium hydride (55.2 g, 2.31 mol, 1.5 equiv) at 0 °C. The resulting solution was stirred for 20 min at 0 °C. Then 2-(bromomethyl) naphthalene (509.7 g, 2.31 mol, 1.5 equiv) was added dropwise with stirring at 0 °C. The reaction mixture was stirred at room temperature for 3 hours. The reaction mixture was quenched with saturated aqueous ammonium chloride. The resulting mixture was diluted with 4 L of ethyl acetate, washed with 2×4 L of water and 2×4 L of saturated aqueous sodium chloride respectively. The organic phase was dried over anhydrous sodium sulfate, filtered and concentrated under reduced pressure. The residue was applied onto a silica gel column with ethyl acetate/petroleum ether (1:5-1:3). 490 g (80%) of 41 was obtained as a yellow oil. MS m/z [M+Na]⁺ (ESI): 423.08.

41 (490 g, 1.23 mol, 1.0 equiv) was dissolved acetic acid (80% in water, 4900 mL) with an inert atmosphere of nitrogen and stirred overnight at room temperature. The resulting mixture was concentrated under reduced pressure and diluted with 5000 mL of ethyl acetate. The resulting mixture was washed with 3×1500 ml of saturated aq. sodium bicarbonate respectively. The organic phase was dried over anhydrous sodium sulfate, filtered and concentrated under reduced pressure. The residue was applied onto a silica gel column with ethyl acetate/petroleum ether (1:5-1:1). 390 g (85%) 42 was obtained as yellow oil. MS m/z [M+Na]⁺ (ESI): 383.21.

To a mixture solution of 42 (195 g, 0.54 mol, 1.0 equiv) in 2000 mL of dichloromethane with an inert atmosphere of argon was added imidazole (146.8 g, 2.16 mol, 4.0 equiv) and tert-butyldiphenylchlorosilane (156 g, 0.55 mol, 1.05 equiv) in order at 0 °C. The reaction mixture was warmed to room temperature and stirred at room temperature for 4 h. The resulting solution was diluted with 5000 mL of dichloromethane and washed with 3×1500 mL of water and 3×1500 mL of saturated aqueous sodium chloride respectively. The organic phase was dried over anhydrous sodium sulfate, filtered and concentrated under reduced pressure. The residue was applied onto a silica gel column with ethyl acetate/petroleum ether (1:10-1:5). 190 g (60%) of 43 was obtained as a yellow oil. MS m/z [M+Na]⁺ (ESI): 621.25. ¹H NMR (300 MHz, DMSO-d₆) δ 7.90-7.85 (m, 1H), 7.79-7.71 (m, 3H), 7.65-7.56 (m, 4H), 7.50-7.45 (m, 2H), 7.43-7.33 (m, 5H), 7.29-7.22 (m, 2H), 5.72 (d, J=3.7 Hz, 1H), 5.06 (d, J=4.8 Hz, 1H), 4.78-4.72 (m, 2H), 4.57 (d, J=11.9 Hz, 1H), 4.19-4.15 (m, 1H), 4.13-3.98 (m, 1H), 3.87-3.94 (m, 1H), 3.83-3.77 (m, 1H), 3.57 (d, J=6.5 Hz, 1H), 1.47 (s, 3H), 1.30 (s, 3H), 0.94 (s, 9H).

To a solution of oxalyl chloride(120.84 g, 0.953 mol, 1.5 equiv) in dichloromethane (2000 mL), was added of dimethyl sulfoxide (78.19 g, 0.95 mol, 3.0 equiv) dropwise with stirring at −78 °C. The resulting solution was stirred for 30 min at −78 °C and was added a solution of 43 (120 g, 158.86 mmol, 1.0 equiv) in dichloromethane (500 mL) dropwise with stirring at −78 °C. The resulting solution was allowed to react, with stirring, for an additional 1.5 hour at −78 °C. Then was added triethylamine (72.24 g, 714.75 mmol, 4.5 equiv) dropwise with stirring at −78 °C. The resulting solution was allowed to react, with stirring, for an additional 2 hours at −78 °C. The resulting solution was diluted with 500 mL of dichloromethane. The resulting mixture was washed with 1×500 mL saturated aqueous sodium bicarbonate, and 1×500 mL saturated aqueous sodium chloride respectively. The mixture was dried over anhydrous sodium sulfate and concentrated under reduced pressure. The residue was applied onto a silica gel column with ethyl acetate/petroleum ether (1:10-1:6). 80 g (85%) of 44 was obtained as a yellow oil. MS m/z [M+Na]⁺ (ESI): 619.57.

To a solution of methyltriphenylphosphonium bromide (96.2 g, 0.269 mol, 2.0 equiv) in tetrahydrofuran (500 mL), was added sodium hydride (6.5 g, 0.269 mol, 2.0 equiv) with an inert atmosphere of nitrogen. The resulting solution was stirred at room temperature for 30 min. The solution of 44 (80 g, 134.7 mmol, 1.00 equiv) in tetrahydrofuran (500 mL) was added. The resulting solution was allowed to react, with stirring, for an additional 3 h at room temperature. The reaction was then quenched by the addition of saturated aqueous ammoniumchloride. The resulting solution was extracted with 4000 mL of ethyl acetate and the organic layers combined. The resulting mixture was washed with water, and saturated aqueous sodium chloride. The mixture was dried over anhydrous sodium sulfate and concentrated under reduced pressure. The residue was applied onto a silica gel column with ethyl acetate/petroleum ether (1:20-1:5). 63 g (80%) of 45 was obtained as a yellow oil. MS m/z [M+Na]⁺ (ESI): 617.60.

To a solution of 45 (125 g, 210.4 mmol, 1.0 equiv) in tetrahydrofuran (1250 mL), was added Borane-methyl sulfide complex (10 M, 42.1 mL, 2.0 equiv) at 0 °C. The resulting solution was stirred 2 h at room temperature. Then was added sodium hydroxide (2N in water 630 mL, 6.0 equiv) at room temperature, added hydrogen peroxide (30%, 143.1 g, 6.0 equiv) dropwise with stirring at room temperature. The resulting solution was allowed to react, with stirring, for an additional 2 h at room temperature. The resulting solution was extracted with 2×2000 mL of dichloromethane and the organic layers combined. The mixture was dried over anhydrous sodium sulfate and concentrated under reduced pressure. The residue was applied onto a silica gel column with ethyl acetate/petroleum ether (1:20-1:5). 73 g (56%) of 46 was obtained as a yellow oil. MS m/z [M+Na]⁺ (ESI): 635.62.

To a solution of triethylamine trihydrofluoride (381.6 g, 2.37 mol, 10.0 equiv) in 730 mL of dry tetrahydrofuran with an inert atmosphere of argon was added triethylamine (391.5 g, 2.37 mol, 10.0 equiv) at room temperature. The resulting solution was stirred at room temperature for 10 min. A solution of 46 (145 g, 236.54 mmol, 1.0 equiv) in 730 mL of dry tetrahydrofuran was added to the resulting solution. The reaction mixture was stirred at room temperature for 12 hours and diluted with 3000 mL of dichloromethane, washed with 2×1000 mL of saturated aqueous sodium bicarbonate and 2×1000 mL of saturated aqueous sodium chloride respectively. The organic layer was dried over anhydrous sodium sulfate, filtered and concentrated under reduced pressure. The residue was purified by Flash-Prep-HPLC with the following conditions: Column, C18 silica gel; mobile phase, water (containing 0.1% FA) and acetonitrile (10% acetonitrile up to 100% in 12 min), UV 254 nm. The fractions were diluted with dichloromethane, dried over anhydrous sodium sulfate and concentrated under reduced pressure. 70 g (80%) of 47 was obtained as a light yellow oil. MS m/z [M+Na]⁺ (ESI): 397.15. 1H NMR (300 MHz, Chloroform-d) δ 7.93-7.80 (m, 4H), 7.57-7.46 (m, 3H), 5.77 (d, J=3.9 Hz, 1H), 5.00 (d, J=11.4 Hz, 1H), 4.77-4.64 (m, 2H), 4.23 (m, 1H), 3.92-3.73 (m, 5H), 2.29 (s, 3H), 2.03-1.87 (m, 1H), 1.64 (s, 3H), 1.40 (s, 3H).

To a solution of 47 (70 g, 187.17 mmol, 1.0 equiv) in 700 mL of dry pyridine with an inert atmosphere of nitrogen was added 4-methylbenzenesulfonyl chloride (107.12 g, 561.51 mmol, 3.0 equiv) at 0 °C. After then, the reaction mixture was stirred at room temperature for 12 hours. The reaction was quenched by the addition of water and concentrated under reduced pressure. The reaction mixture was diluted with 3000 mL of dichloromethane and washed with 1×1000 mL saturated sodium bicarbonate and 2×1000 mL saturated sodium chloride respectively. The organic phase was dried over anhydrous sodium sulfate. The solid was filtered out and concentrated under reduced pressure. The crude product was purified by Flash-Prep-HPLC with the following conditions (IntelFlash-1): Column, C18 silica gel; mobile phase, water (containing 0.04% NH₄HCO₃) and acetonitrile (10% acetonitrile up to 100% in 15 min and hold 100% for 5 min); Detector, UV 254 nm. 90 g (70%) of 48 was obtained as a light yellow solid. [M+Na+]⁺ (ESI): 705.81

To a solution of diethyl ((phenylthio)methyl)phosphonate (22.62 g, 87.00 mmol, 2.00 equiv) in 150 mL of dry tetrahydrofuran with an inert atmosphere of nitrogen, was added of butyllithium (2 M, 43.50 mL 2.00 equiv) dropwise with stirring at −78 °C. The resulting solution was stirred at −30 °C for 2 hours. The solution of 48 (30 g, 43.99 mmol, 1.00 equiv) in 150 mL of tetrahydrofuran was added dropwise with stirring at −78 °C. The resulting solution was stirred at room temperature for 12 hours. The resulting solution was extracted with ethyl acetate and the organic layers combined. The organic layer was washed with 1×500 mL water and 2×500 mL saturated sodium chloride respectively. The mixture was dried over anhydrous sodium sulfate and concentrated under reduced pressure. The crude product was purified by Flash-Prep-HPLC with the following conditions (IntelFlash-1): Column, C18 silica gel; mobile phase, water (containing 0.04% NH₄HCO₃) and acetonitrile (30% acetonitrile up to 100% in 10 min and hold 100% for 5 min); Detector, UV 254 nm. 12.5 g (50%) of 201 was obtained as a light yellow solid. [M+H+]⁺ (ESI): 599.66

To a solution of 201 (15 g, 25.08 mmol, 1.0 equiv) in 300 mL of toluene with an inert atmosphere of nitrogen was added 2,2′-Azobis(2-methylpropionitrile) (3.29 g, 20.07 mmol, 0.4 equiv) and tributylstannane (14.6 g, 50.16 mmol, 2.0 equiv) at room temperature. The resulting solution was stirred at 110 °C for 1 hours. The resulting mixture was concentrated under reduced pressure. The crude product was purified by Flash-Prep-HPLC with the following conditions: Column, C18 silica gel; mobile phase, water (containing 0.04% NH₄HCO₃) and acetonitrile (30% acetonitrile up to 100% in 10 min and hold 100% for 5 min); Detector, UV 254 nm. 8.5 g (65%) of 91 was obtained as a light yellow solid. [M+H+]⁺ (ESI): 491.15. 1H NMR (400 MHz, DMSO-d6) δ 7.96-7.82 (m, 4H), 7.57-7.43 (m, 3H), 5.80-5.71 (m, 1H), 4.85-4.72 (m, 2H), 4.64 (d, J=12.2 Hz, 1H), 4.02-3.78 (m, 5H), 3.51-3.44 (m, 1H), 2.65-2.58 (m, 2H), 2.21-1.85 (m, 4H), 1.48 (d, J=5.0 Hz, 3H), 1.31 (d, J=3.1 Hz, 3H), 1.19 (m, 6H). 31P NMR (162 MHz, DMSO) δ 33.08, 30.36.

To a solution of 91 (17 g, 34.69 mmol, 1.0 equiv) in 170 mL of acetic acid, with an inert atmosphere of nitrogen was added acetyl acetate (35.4 g, 346.94 mmol, 10.0 equiv), and sulfuric acid (679.9 mg, 6.94 mmol, 0.2 equiv). The resulting solution was stirred at room temperature for 2 hours. The reaction was then quenched by the addition of ice water. The resulting solution was extracted with ethyl acetate. The resulting mixture was washed with water and saturated aqueous sodium chloride. The mixture was dried over anhydrous sodium sulfate and concentrated under reduced pressure. The crude product was purified by Flash-Prep-HPLC with the following conditions: Column, C18 silica gel; mobile phase, water and acetonitrile (20% acetonitrile up to 100% in 10 min and hold 100% for 5 min); Detector, UV 254 nm. 14 g (75%) of 82 was obtained as a light yellow solid. MS m/z [M+H]⁺ (ESI):535.72.

To a solution of Uracil (5.87 g, 52.43 mmol, 2.0 equiv) in 140 mL of acetonitrile with an inert atmosphere of nitrogen, was added N,O-Bis(trimethylsilyl)acetamide (23.95 g, 117.98 mmol, 4.5 equiv) at room temperature. The resulting solution was stirred at 60 °C for 0.5 hour. To this was added 82 (14 g, 26.22 mmol, 1.00 equiv) at 0 °C, and then perchlorostannane (8.86 g, 34.08 mmol, 1.3 equiv) was added dropwise with stirring at 0 °C. The resulting solution was allowed to react, with stirring, for an additional 1 h at 60 °C. The reaction mixture was cooled to 0 °C, quenched by the addition of 100 mL saturated aqueous sodium bicarbonate. The resulting solution was extracted with 2×300 mL ethyl acetate and the organic layers combined and dried over anhydrous sodium sulfate and concentrated under reduced pressure. The crude product was purified by Flash-Prep-HPLC with the following conditions: Column, C18 silica gel; mobile phase, water (containing 0.04% NH₄HCO₃) and acetonitrile (45% acetonitrile up to 100% in 10 min and hold 100% for 5 min); Detector, UV 254 nm. 9 g (60%) of 83 was obtained as a white solid. MS m/z [M+H]⁺ (ESI):587.10. 1H NMR (300 MHz, DMSO-d6) δ 11.43 (s, 1H), 7.93 (d, J=8.7 Hz, 3H), 7.84 (s, 1H), 7.68 (d, J=13.4 Hz, 1H), 7.54 (d, J=9.3 Hz, 2H), 7.47 (d, J=8.5 Hz, 1H), 5.79 (dd, J=12.3, 3.8 Hz, 1H), 5.68 (d, J=8.0 Hz, 1H), 5.49-5.42 (m, 1H), 4.68 (d, J=2.7 Hz, 2H), 4.11 (t, J=6.3 Hz, 1H), 3.98-3.90 (m, 5H), 2.78-2.56 (m, 2H), 2.10 (s, 4H), 2.04-1.98 (m, 1H), 1.24 (d, J=7.0 Hz, 2H), 1.21-1.14 (m, 6H). 31P NMR (121 MHz, DMSO) δ 32.76, 30.94, 30.15, 30.14.

To a solution of 83 (9 g, 15.36 mmol, 1.0 equiv) in 90 mL of ethanol with an inert atmosphere of nitrogen, was added Sodium methanolate (1.24 g, 23.04 mmol, 1.5 equiv) at 0 °C. The resulting solution was stirred at room temperature for 2 hours. Then the mixture was adjusted to pH=7 by Amberlite IR-120 (H+). The resulting mixture was filtered and the filter cake washed with 3×50 mL ethanol. The filtrate was concentrated under reduced pressure. This 9 g crude product was used in the next step without further purification. MS m/z [M+H]⁺ (ESI):549.32.

To a solution of 84 (15.36 mmol, 1.0 equiv) in 70 mL of tetrahydrofuran with an inert atmosphere of nitrogen, was added sodium hydride (737.28 mg, 30.72 mmol, 2.0 equiv) at 0 °C. The mixture was stirred at 0 °C for 30 minute. A solution of methyl Iodide (4.33 g, 30.72 mmol, 2.0 equiv) in 20 mL tetrahydrofuran was added dropwise at 0 °C. The resulting solution was stirred at room temperature for 2 hours. The reaction was quenched with 50 mL water. The resulting solution was extracted with 200 mL ethyl acetate and the organic phase combined. The organic phase was washed with water then dried over anhydrous sodium sulfate and concentrated under reduced pressure. The crude product was purified by Flash-Prep-HPLC with the following conditions (IntelFlash-1): Column, C18 silica gel; mobile phase, water (containing 0.04% NH₄HCO₃) and acetonitrile (20% acetonitrile up to 100% in 25 min and hold 100% for 5 min); Detector, UV 254 nm. 5 g (60% over two steps) of 416 was obtained as a white solid. MS m/z [M+H]⁺ (ESI):559.41.

To a solution of 416 (6.0 g, 14.33 mmol, 1.0 equiv) in 72 mL of dichloromethane and 8 mL of water, with an inert atmosphere of nitrogen was added 2,3-Dichloro-5,6-dicyano-1,4-benzo-quinone (4.6 g, 20.06 mmol, 1.4 equiv) at room temperature. The resulting solution was stirred at room temperature for 1 hour. The reaction mixture was diluted with 40 mL of water and extracted with 200 mL dichloromethane. The water phase was concentrated under reduced pressure. The crude product was purified by Flash-Prep-HPLC with the following conditions: Column, C18 silica gel; mobile phase, water (containing 0.04% NH₄HCO₃) and acetonitrile (5% acetonitrile up to 50% in 15 min and hold 100% for 5 min); Detector, UV 254 nm. 3 g (60%) of the mixture was obtained as a white solid and separated by SFC. 1.5 g of 417 was obtained as white solid. MS m/z [M+H]⁺ (ESI):419.00. ³¹P NMR (162 MHz, DMSO) δ 30.27. ¹H NMR (400 MHz, DMSO-d6) δ 11.39 (d, J=2.3 Hz, 1H), 7.60 (d, J=8.0 Hz, 1H), 5.77-5.74 (m, 1H), 5.66 (dd, J=8.1, 2.2 Hz, 1H), 5.17 (s, 1H) 4.07-3.89 (m, 4H), 3.89-3.79 (m, 2H), 3.68 (dd, J=7.4, 5.1 Hz, 1H), 3.34 (s, 3H), 2.69-2.61 (m, 2H), 2.15-1.97 (m, 4H), 1.20 (t, J=7.1 Hz, 6H).

1.2 g of 417S (used for 100) was obtained as white solid. MS m/z [M+H]⁺ (ESI):419.00. ³¹P NMR (162 MHz, DMSO) δ 32.80 ¹H NMR (400 MHz, DMSO-d6) δ 11.39 (d, J=2.2 Hz, 1H), 7.57 (d, J=8.1 Hz, 1H), 5.79-5.76 (m, 1H), 5.67 (dd, J=8.0, 2.2 Hz, 1H), 5.17 (s, 1H), 4.07-3.90 (m, 4H), 3.90-3.77 (m, 3H), 3.34 (s, 3H), 2.63 (q, J=8.8, 7.4 Hz, 2H), 2.26-2.02 (m, 4H), 1.22 (td, J=7.1, 0.8 Hz, 6H).

To a solution of 417 (1.5 g, 3.6 mmol, 1.0 equiv) in 15 mL of dichloromethane with an inert atmosphere of argon was added 2-Cyanoethyl N,N,N′,N′-tetraisopropylphosphorodiamidite (1.41 g, 4.7 mmol, 1.3 equiv) and 4,5-Dicyanoimidazole (467.3 mg, 3.9 mmol, 1.1 equiv) in order at room temperature. The reaction mixture was stirred at room temperature for 1 hour. The reacting solution was diluted with 100 mL of dichloromethane, washed with 1×20 mL saturated aqueous sodium bicarbonate and 1×20 mL saturated aqueous sodium chloride respectively. The organic phase was dried over anhydrous sodium sulfate, filtered and concentrated till no residual solvent left under reduced pressure. The residue was purified by Flash-Prep-HPLC with the following conditions: Column, C18 silica gel; mobile phase, water (containing 0.04% NH₄HCO₃) and acetonitrile (20% acetonitrile up to 100% in 10 min and 100% acetonitrile hold 5 min). The organic phase layer was separated. The aqueous phase was extracted with 3×50 mL of dichloromethane. The organic phase was combined and dried over anhydrous sodium sulfate. The solid was filtered out. The filtrate was concentrated at 25 °C, obtained the product which was dried for 6 hours under reduced pressure at 25 °C. 1.38 g (64%) 2-0 was obtained as a white solid. MS m/z [M−H]⁻ (ESI):617.15. ¹H NMR (300 MHz, Chloroform-d) δ 9.24 (s, 1H), 7.40-7.36 (m, 1H), 5.82-5.72 (m, 2H), 4.14-4.03 (m, 5H), 4.01-3.81 (m, 3H), 3.76-3.56 (m, 3H), 3.51 (m, 3H), 2.80-2.59 (m, 4H), 2.33-2.22 (m, 4H), 1.33-1.28 (m, 6H), 1.22-1.17 (m, 12H). ³¹P NMR (121 MHz, CDCl₃) δ 149.84, 149.66, 29.89, 29.55.

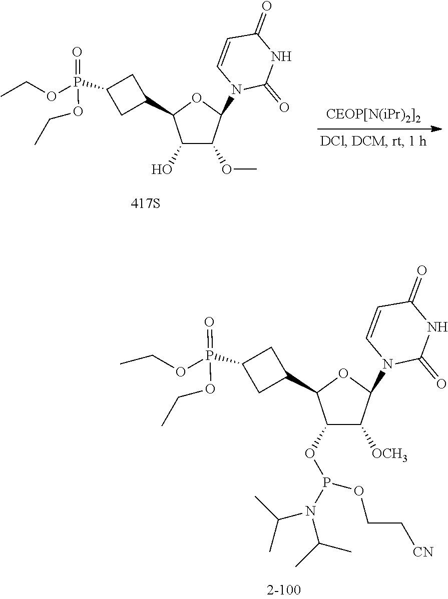

To a solution of 417 (1.2 g, 2.9 mmol, 1.0 equiv) in 20 mL of dichloromethane with an inert atmosphere of argon was added 2-Cyanoethyl N,N,N′,N′-tetraisopropylphosphorodiamidite (1.1 g, 3.8 mmol, 1.3 equiv) and 4,5-Dicyanoimidazole (376.4 mg, 3.2 mmol, 1.1 equiv) in order at room temperature. The reaction mixture was stirred at room temperature for 1 hour. The reacting solution was diluted with 20 mL of dichloromethane, washed with saturated aqueous sodium bicarbonate and saturated aqueous sodium chloride respectively. The organic phase was dried over anhydrous sodium sulfate, filtered and concentrated till no residual solvent left under reduced pressure. The residue was purified by Flash-Prep-HPLC with the following conditions: Column, C18 silica gel; mobile phase, water (containing 0.04% NH₄HCO₃) and acetonitrile (20% acetonitrile up to 100% in 10 min and 100% acetonitrile hold 5 min). The organic phase layer was separated. The aqueous phase was extracted with 3×50 mL of dichloromethane. The organic phase was combined and dried over anhydrous sodium sulfate. The solid was filtered out. The filtrate was concentrated at 25 °C, obtained the product which was dried for 4 hours under reduced pressure at 25 °C. 990.9 mg (70%) 2-100 was obtained as a white solid. MS m/z [M−H]⁻ (ESI):617.25. ¹H NMR (300 MHz, Chloroform-d) δ 9.28 (s, 1H), 7.30-7.23 (m, 1H), 5.85 (dd, J=7.7, 3.2 Hz, 1H), 5.77 (dd, J=8.1, 1.2 Hz, 1H), 4.19-4.05 (m, 5H), 4.02-3.55 (m, 6H), 3.51 (d, J=11.7 Hz, 3H), 2.85-2.71 (m, 1H), 2.69-2.58 (m, 3H), 2.56-2.18 (m, 4H), 1.37-1.27 (m, 6H), 1.25-1.11 (m, 12H). ³¹P NMR (121 MHz, CDCl₃) δ 149.70, 149.69, 32.96, 32.65.

### Example 2-3

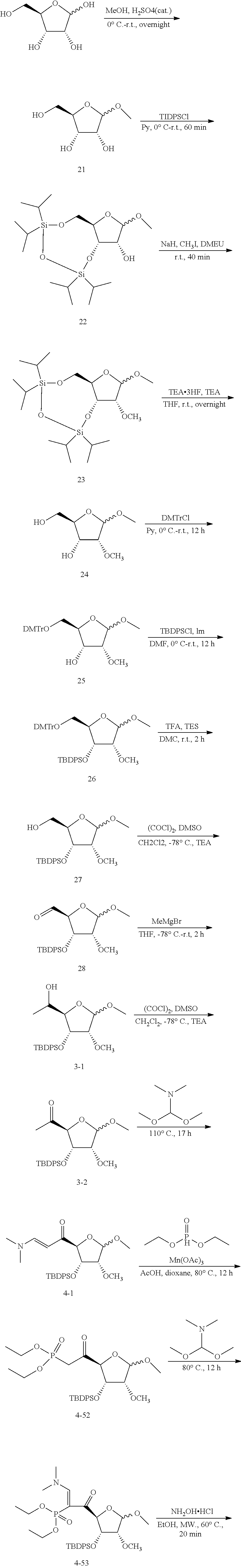

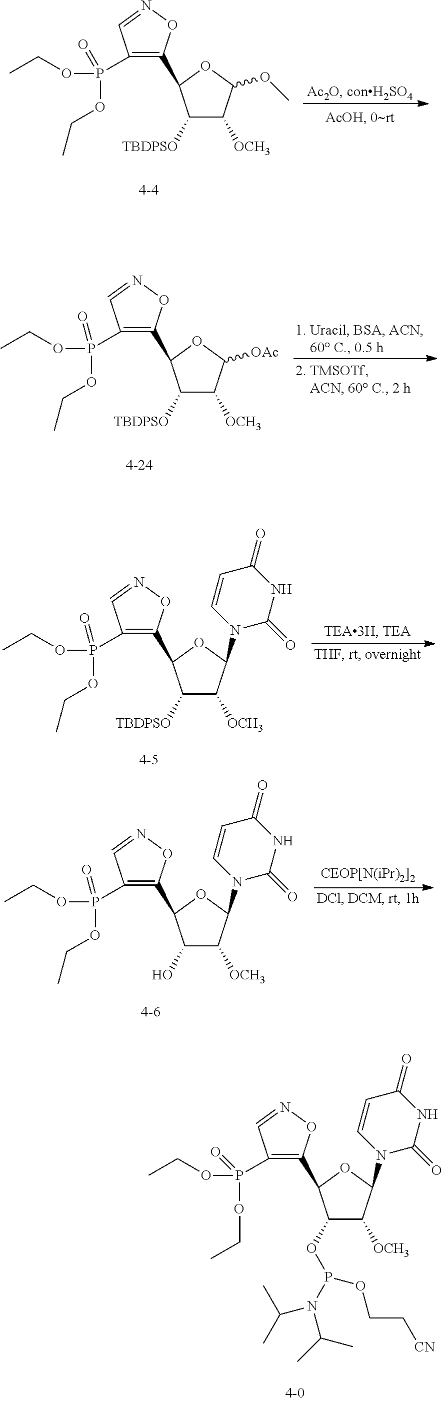

To a solution of (3R,4S,5R)-5-(hydroxymethyl) tetrahydrofuran-2,3,4-triol (600 g, 3.997 mol, 1.0 equiv) in 6 L of methanol with an inert atmosphere of argon was slowly added Sulfuric acid (39.19 g, 0.4 mol, 0.1 equiv) at 0 °C. The reaction mixture was allowed to warm to room temperature and stirred overnight. The mixture was adjusted to pH 7 with sodium bicarbonate, then filtered and concentrated under reduced pressure to afford 21 (520 g, crude) as yellow oil. MS m/z [M+H]⁺ (ESI): 165.07. This crude product was used in the next step without further purification.

To a solution of 21 (260 g, 1.584 mol, 1.0 equiv) in 2.6 L of pyridine with an inert atmosphere of argon was added 1,3-dichloro-1,1,3,3-tetraisopropyldisiloxane (549.5 g, 1.742 mol, 1.1 equiv) at 0 °C. The mixture was stirred at room temperature for 1 h. The resulting solution was extracted with 3×4 L of ethyl acetate and the organic layers combined. The organic layer was washed with 2×500 mL of water and 2×4 L of saturated sodium chloride respectively. The mixture was dried over anhydrous sodium sulfate and concentrated under reduced pressure. The residue was dissolved in 100 mL of ethyl acetate and evaporated to dryness. The residue was applied onto a silica gel column with ethyl acetate/petroleum ether (1:5-1:3). 195 g (33%) of 22 was obtained as a yellow oil. MS m/z [M+H]⁺ (ESI): 407.23.

To a mixture solution of 22 (195 g, 0.48 mol, 1.0 equiv) in 2 L of 1,3-Dimethyl-2-imidazolidinone and 1 L of iodomethane with an inert atmosphere of argon was added sodium hydride (23.1 g, 0.959 mol, 2.0 equiv). The resulting solution was stirred for 40 min at room temperature. The resulting solution was diluted with dichloromethane and washed with water. The organic layer was dried over anhydrous sodium sulfate, filtered and concentrated under reduced pressure. The crude product was purified by column chromatography (Silica gel, ethyl acetate/petroleum ether=1:5 to 1:1) to afford 23 (390 g, crude) as a yellow oil. MS m/z [M+H]⁺ (ESI):421.25. H NMR (400 MHz, Chloroform-d) δ 4.77 (s, 1H), 4.50 (m, 1H), 4.01 (m, 2H), 3.94-3.84 (m, 1H), 3.61 (d, J=4.3 Hz, 1H), 3.59 (s, 3H), 3.34 (s, 3H), 1.15-1.02 (m, 28H). This crude product was used in the next step without further purification.

To a solution of triethylamine trihydrofluoride (3 kg, 18.541 mol, 10.0 equiv) in 5 L of dry tetrahydrofuran with an inert atmosphere of argon was added triethylamine (3.7 kg, 37.082 mol, 20.0 equiv) at room temperature. The resulting solution was stirred at room temperature for 10 min. A solution of 23 (780 g, 1.854 mol, 1.0 equiv) in 1 L of dry tetrahydrofuran was added to the resulting solution. The reaction mixture was stirred overnight at room temperature and diluted with 3 L of dichloromethane, washed with 2×1 L of saturated aqueous sodium bicarbonate and 2×1 L of saturated aqueous sodium chloride respectively. The organic layer was dried over anhydrous sodium sulfate, filtered and concentrated under reduced pressure. The crude product was purified by column chromatography (Silica gel, ethyl acetate/petroleum ether=1:5 to 3:1) to afford 24 (280 g) as a yellow oil. MS m/z [M+H]⁺ (ESI): 179.11.

To a solution of crude 24 (140 g, 0.786 mol, 1.0 equiv) in 1.4 L of pyridine with an inert atmosphere of argon was added 4,4′-(chloro(phenyl)methylene)bis(methoxybenzene) (292.8 g, 0.865 mol, 1.1 equiv) at 0 °C. The mixture solution was stirred at room temperature for 12 hours. The resulting solution was diluted with 3000 mL dichloromethane and washed with 2×1000 mL water. The organic phase was dried over anhydrous sodium sulfate, filtered and concentrated under reduced pressure. The residue was applied onto a silica gel column with ethyl acetate/petroleum ether (1:50-1:1). 230 g (61%) of 25 was obtained as a yellow oil. MS m/z [M+H]⁺ (ESI): 481.23.

To a solution of 25 (460 g, 0.957 mol, 1.00 equiv) in 4.6 L of N,N-Dimethylformamide with an inert atmosphere of nitrogen was added tert-Butylchlorodiphenylsilane (289.4 g, 1.053 mol, 1.1 equiv) and imidazole (162.9 g, 2.393 mol, 2.5 equiv) at room temperature. The resulting solution was stirred at room temperature for 12 hours. The organic layer was washed with water and extracted with 3×4 L ethyl acetate. The organic layers were dried over anhydrous sodium sulfate and concentrated under reduced pressure. The residue was applied onto a silica gel column with ethyl acetate/petroleum ether (1:20-1:5). 520 g (72%) 26 as a yellow oil. MS m/z [M+H]⁺ (ESI):719.35.

To a solution of 26 (520 g, 166.910 mmol, 1.0 equiv) in 5.2 L dichloromethane was added triethylsilane (500 mL) at room temperature. Then trifluoroacetic acid (500 mL) was added at 0 °C and the resulting solution was stirred at room temperature for 2 hours. The reaction was quenched by the addition of 5 L saturated sodium bicarbonate and extracted with 3×2 L of dichloromethane. The organic layers were combined, dried over anhydrous sodium sulfate, filtered and concentrated under reduced pressure. The residue was applied onto a silica gel column with ethyl acetate/petroleum ether (1:10-5:1). The fractions of desired product were concentrated under reduced pressure. 260 g (82%) of 27 was obtained as a yellow oil. MS m/z [M+NH₄]⁺ (ESI): 434.35. H NMR (400 MHz, DMSO-d6) δ 7.81-7.34 (m, 10H), 4.79 (d, J=1.5 Hz, 1H), 4.65 (s, 1H), 4.16 (m, 1H), 3.91 (m, 1H), 3.44 (dd, J=11.6, 3.6 Hz, 1H), 3.22 (m, 1H), 3.19 (s, 3H), 3.11 (s, 3H), 3.03 (m, 1H), 1.03 (s, 9H).

To a solution of oxalyl chloride (47.3 g, 372.6 mmol, 2.0 equiv) in 1.3 L of dichloromethane with an inert atmosphere of nitrogen, was added Dimethyl sulfoxide (43.7 g, 558.9 mmol, 3.0 equiv) at −78 °C. The resulting solution was stirred for 0.5 h at −78 °C then added a solution of 27 (75 g, 186.3 mmol, 1.0 equiv) in 750 mL of dichloromethane dropwise with stirring at −78 °C. The resulting solution was stirred at −78 °C for 3 hours. Then triethylamine (84.8 g, 838.4 mmol, 4.5 equiv) was added dropwise with stirring at for 0.5 h at −78 °C. The resulting solution was diluted with 2000 mL of dichloromethane. The resulting mixture was washed with 1×1 L saturated aqueous sodium bicarbonate, and 1×1 L saturated aqueous sodium chloride respectively. The organic phase was dried over anhydrous sodium sulfate, filtered and concentrated under reduced pressure. The process above was repeated 2 times, 120 g (crude) 28 was obtained as yellow oil. This crude product was used in the next step without further purification. MS m/z [M+H]⁺ (ESI): 415.21.

To a solution of 28 (120 g, 289.5 mmol, 1.0 equiv) in 1.2 L of tetrahydrofuran with an inert atmosphere of nitrogen, methylmagnesium bromide (103.549 g, 868.4 mmol, 3.0 equiv) at −78 °C. The resulting solution was stirred at −78 °C for 2 hours. The resulting solution was diluted with 3 L dichloromethane and washed with 2×1000 mL water. The organic phase was dried over anhydrous sodium sulfate and concentrated under reduced pressure. The residue was applied onto a silica gel column with ethyl acetate/petroleum ether (1:10-5:1). The fractions of desired product were concentrated under reduced pressure. 86 g (62%) of 3-1 was obtained as a yellow oil. MS m/z [M+H]⁺ (ESI): 431.25.

To a solution of oxalyl chloride (50.7 g, 399.4 mmol, 2.0 equiv) in 860 ml of dichloromethane with an inert atmosphere of nitrogen, was added dimethyl sulfoxide (46.8 g, 599.12 mmol, 3.0 equiv) at −78 °C. The resulting solution was stirred for 0.5 h at −78 °C. And the 3-1(86 g, 199.7 mmol, 1.00 equiv) was added. The resulting solution was stirred at −78 °C for 3 hours. Then triethylamine (90.9 g, 898.7 mmol, 4.5 equiv) was added dropwise with stirring at −78 °C. The resulting solution was stirred for 0.5 h at −78 °C. The resulting solution was diluted with 1 L dichloromethane and washed with 3×600 mL water. The organic layer was dried over anhydrous sodium sulfate, filtered and concentrated under reduced pressure. The residue was applied onto a silica gel column with ethyl acetate/petroleum ether (1:50-3:1). The fractions of desired product were concentrated under reduced pressure. 72 g (80%) of 3-2 was obtained as a yellow oil. MS m/z [M+H]⁺ (ESI): 429.22. H NMR (300 MHz, DMSO-d6) δ 7.71-7.30 (m, 10H), 4.94 (d, J=1.2 Hz, 1H), 4.36 (m, 1H), 4.27 (d, J=6.8 Hz, 1H), 3.12 (s, 3H), 3.01 (m, 1H), 1.99 (d, J=4.9 Hz, 3H), 1.01 (s, 9H).

To a solution of 3-2 (72 g, 168.0 mmol, 1.0 equiv) in 720 mL of N,N-Dimethylformamide dimethyl acetal with an inert atmosphere of nitrogen. The resulting solution was stirred at 110 °C for 17 hours then diluted with 1 L dichloromethane and washed with 2×600 mL water. The resulting mixture was concentrated under reduced pressure. The residue was applied onto a silica gel column with ethyl acetate/petroleum ether (1:50-5:1). The fractions of desired product were concentrated under reduced pressure. 60 g (70%) of 4-1 was obtained as a yellow oil. MS m/z [M+H]⁺ (ESI): 484.26.

To a solution of 4-1 (30 g, 62.026 mmol, 1.0 equiv) in 100 mL of acetic acid and 200 mL of 1.4-dioxane with an inert atmosphere of nitrogen, was added diethyl phosphonate (17.1 g, 124.1 mmol, 2.0 equiv), manganese triacetate dihydrate (49.9 g, 186.1 mmol, 3.0 equiv) at room temperature. The resulting solution was stirred at 80 °C for 20 min. The resulting solution was extracted with 3×500 mL of ethyl acetate and the organic layers combined. The organic layers were washed with 2×200 mL of water and 2×200 mL of saturated sodium chloride respectively. The mixture was dried over anhydrous sodium sulfate and concentrated under reduced pressure. The process above was repeated 2 times. The crude product was purified by Flash-Prep-HPLC with the following conditions (IntelFlash-1): Column, C18 silica gel; mobile phase, water and acetonitrile (30% acetonitrile up to 100% in 10 min and hold 100% for 5 min); Detector, UV 254 nm. This resulted in 38 g (52%) of 4-52 as a yellow oil. MS m/z [M+H]⁺ (ESI): 565.25.

To a solution of 4-52 (38 g, 67.3 mmol, 1.0 equiv) in 380 mL of 1,1-dimethoxy-N,N-dimethylmethanamine. The resulting solution was stirred at 80 °C for 12 hours. The resulting solution was diluted with 500 mL dichloromethane and washed with 2×300 mL water. The crude product was purified by Flash-Prep-HPLC with the following conditions (IntelFlash-1): Column, C18 silica gel; mobile phase, water (containing 0.04% NH₄HCO₃) and acetonitrile (15% acetonitrile up to 100% in 25 min and hold 100% for 5 min); Detector, UV 254 nm. 27 g (62%) of 4-53 was obtained as a yellow solid. MS m/z [M+H]⁺ (ESI): 620.30.

To a solution of 4-53 (27 g, 43.56 mmol, 1.0 equiv) in 270 mL of ethanol with an inert atmosphere of nitrogen, was added hydroxylamine hydrochloride (9.1 g, 130.7 mmol, 3.0 equiv) and pyridine (34.5 g, 435.6 mmol, 10.0 equiv) at room temperature. The final reaction mixture was stirred at 60 °C for 1 hour. The resulting solution was diluted with 400 mL dichloromethane and washed with 2×200 mL water. The crude product was purified by Flash-Prep-HPLC with the following conditions (IntelFlash-1): Column, C18 silica gel; mobile phase, water and acetonitrile (30% acetonitrile up to 100% in 10 min and hold 100% for 5 min); Detector, UV 254 nm. 16 g (59.2%) of 4-4 was obtained as a yellow oil. MS m/z [M−H]⁻ (ESI): 588.25.

To a solution of 4-4 (16 g, 42.395 mmol, 1.0 equiv) in 160 mL of acetic acid and 80 mL of acetic anhydride was added sulfuric acid (133.1 mg, 1.357 mmol, 0.05 equiv) dropwise with stirring at 0 °C. The resulting solution was stirred at room temperature for 1 hour then diluted with 300 mL dichloromethane and washed with 2×100 mL water. The crude product was purified by Flash-Prep-HPLC with the following conditions (IntelFlash-1): Column, C18 silica gel; mobile phase, water (containing 0.04% NH₄HCO₃) and acetonitrile (15% acetonitrile up to 100% in 25 min and hold 100% for 5 min); Detector, UV 254 nm. 12 g (68%) of 4-24 was obtained as a yellow oil. MS m/z [M+H]⁺ (ESI): 618.24.

To a solution of uracil (0.544 g, 4.86 mmol, 1.5 equiv) in 80 mL of acetonitrile with an inert atmosphere of nitrogen, was added N, O-Bis (trimethylsilyl)acetamide (2.305 g, 11.3 mmol, 3.5 equiv) at room temperature. The resulting solution was stirred at 60 °C for 0.5 hour. The reaction mixture was cooled to 0 °C and 4-4 (2 g, 3.24 mmol, 1.0 equiv) was added. Then trimethylsilyl trifluoromethanesulfonate (1.44 g, 6.48 mmol, 2.0 equiv) was added dropwise with stirring at 0 °C. The resulting solution was allowed to react, with stirring at 60 °C for 1 hour. The resulting solution was diluted with dichloromethane and washed with water. The process above was repeated 6 times. The crude product was purified by Hash-Prep-HPLC with the following conditions (IntelFlash-1): Column, C18 silica gel; mobile phase, water (containing 0.04% NH₄HCO₃) and acetonitrile (30% acetonitrile up to 100% in 10 min and hold 100% for 5 min); Detector, UV 254 nm. 3.28 g (24%) of 4-5 was obtained as an off-white solid. MS m/z [M+H]⁺ (ESI): 670.25.

To a solution of triethylamine trihydrofluoride (7.9 g, 49.0 mmol, 10.0 equiv) in 20 mL of dry tetrahydrofuran with an inert atmosphere of argon was added triethylamine (9.9 g, 97.9 mmol, 20.0 equiv) at room temperature. The resulting solution was stirred at room temperature for 10 min. A solution of 4-5 (3.28 g, 4.9 mmol, 1.0 equiv) in 12 mL of dry tetrahydrofuran was added to the resulting solution. The reaction mixture was stirred overnight at room temperature and diluted with 200 mL of dichloromethane, washed with 2×200 mL of saturated aqueous sodium bicarbonate and 2×200 mL of saturated aqueous sodium chloride respectively. The organic phase was dried over anhydrous sodium sulfate, filtered and concentrated under reduced pressure. The crude product was purified by Flash-Prep-HPLC with the following conditions (IntelFlash-1): Column, C18 silica gel; mobile phase, water (containing 0.04% NH₄HCO₃) and acetonitrile (30% acetonitrile up to 100% in 10 min and hold 100% for 5 min); Detector, UV 254 nm. 1.3 g (62%) of 4-6 was obtained as a yellow oil. MS m/z [M+H]⁺ (ESI): 432.15. H NMR (400 MHz, DMSO-d6) δ 11.50-11.29 (m, 1H), 8.89 (s, 1H), 7.76 (d, J=8.1 Hz, 1H), 6.45 (d, J=5.0 Hz, 1H), 6.05 (d, J=6.0 Hz, 1H), 5.66 (m, 1H), 5.52 (d, J=6.1 Hz, 1H), 4.68 (m, 1H), 4.16 (t, J=4.8 Hz, 1H), 4.07 (m, 3.7 Hz, 4H), 3.36 (s, 3H), 1.24 (m, 6H).

To a solution of 4-6 (1 g, 2.318 mmol, 1.0 equiv) in 10 mL of dichloromethane was added 2-Cyanoethyl N,N,N′,N′-tetraisopropylphosphorodiamidite (838.6 mg, 2.782 mmol, 1.2 equiv) and 4,5-Dicyanoimidazole (273.8 mg, 2.318 mmol, 1.0 equiv) at room temperature. The resulting solution was stirred at room temperature for 1 hour. The reacting solution was diluted with 300 ml of dichloromethane, washed with 2×100 mL of saturated aqueous sodium bicarbonate and 1×100 mL of saturated aqueous sodium chloride respectively. The organic phase was dried over anhydrous sodium sulfate, filtered and concentrated till no residual solvent left under reduced pressure. The crude product was purified by Flash-Prep-HPLC with the following conditions (IntelFlash-1): Column, C18 silica gel; mobile phase, water (containing 0.04% NH₄HCO₃) and acetonitrile (30% acetonitrile up to 100% in 15 min and hold 100% for 5 min); Detector, UV 254 nm. The fraction was dilute with equal volume of dichloromethane. The organic phase layer was separated. The aqueous phase was extracted with 3×50 mL of dichloromethane. The organic phase was combined and dried over anhydrous sodium sulfate. The solid was filtered out. The filtrate was concentrated at 25 °C. 840.5 mg (57%) of 4-0 was obtained as a white solid. MS m/z [M−H]⁻ (ESI): 630.20. H NMR (300 MHz, DMSO-d6) δ 11.43 (s, 1H), 8.90 (s, 1H), 7.65 (m, 1H), 6.48 (d, J=4.4 Hz, 1H), 5.80-5.56 (m, 2H), 4.95 (m, 1H), 4.24 (m, 1H), 4.16-3.95 (m, 4H), 3.82-3.63 (m, 2H), 3.62-3.40 (m, 2H), 3.37 (d, J=3.5 Hz, 3H), 2.76 (m, 2H), 1.32-1.16 (m, 7H), 1.09 (t, J=6.8 Hz, 8H), 0.96 (d, J=6.8 Hz, 3H). P NMR (121 MHz, DMSO) δ 150.00, 149.96, 7.37, 7.35.

### Example 2-4

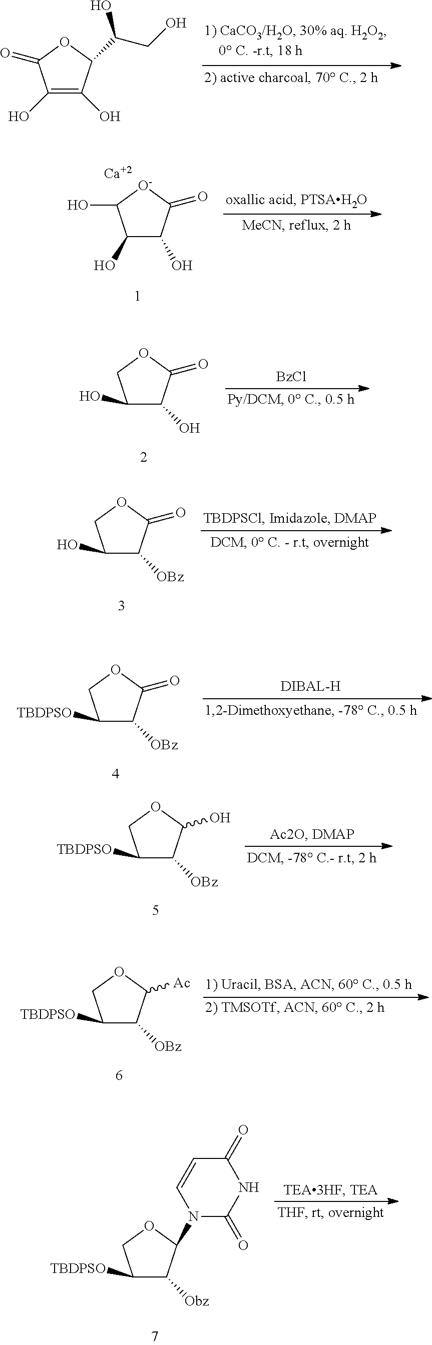

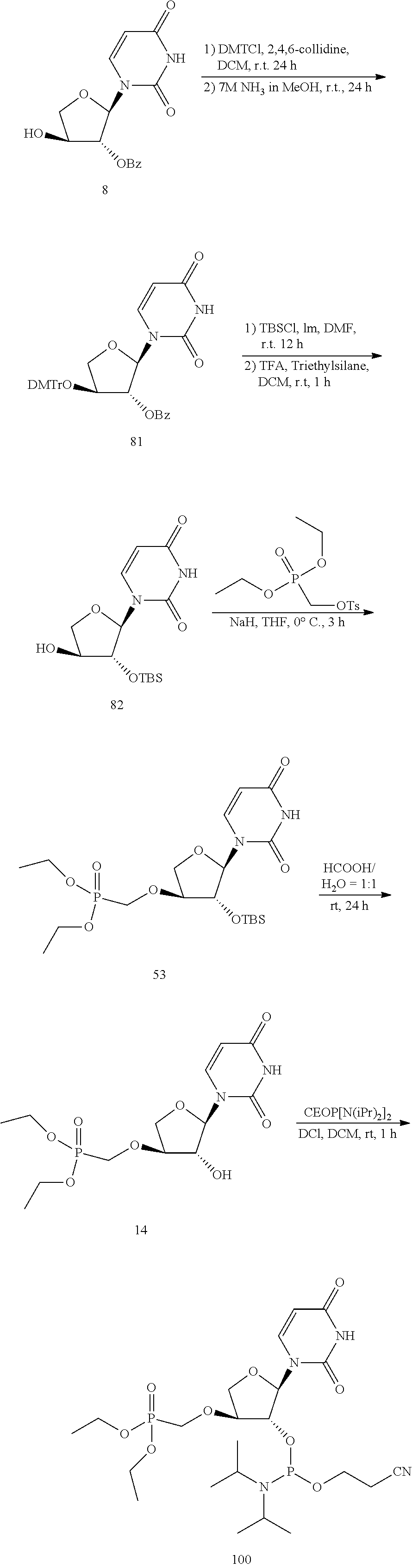

To a solution of L-ascorbic acid (125 g, 0.71 mol, 1.0 equiv) in 1 L of water with an inert atmosphere of argon was slowly added calcium carbonate (125 g, 1.25 mol, 1.76 equiv) over 30 min. The resulting solution was added hydrogen peroxide (250 mL, 30% aq.) dropwise over 1 h with stirring at 0 °C. The reaction mixture was allowed to warm to room temperature and stirred overnight. After then, the reaction mixture was filtered and the filter cake was washed with 2×100 mL of water. The filtrate was treated with activated carbon (25 g), then heated to 70 °C. The hot suspension was filtered and the solid material was washed with 2×50 mL of water. The filtrate was combined, crystallized by the addition of 2 volume equivalents of methanol while stirring for 16 h at 4 °C. The solid material was filtered, washed with 2×50 mL of methanol and dried under high vacuum at 40 °C. 88.1 g (75%) 1 was obtained as a white solid. No MS signal. ¹H NMR (400 MHz, D₂O): δ 3.98 (d, J=2.2 Hz, 1H), 3.88-3.72 (m, 1H), 3.62-3.49 (m, 2H).

To a solution of 1 (100 g, 0.30 mol, 1.0 equiv) in 500 mL of dry acetonitrile with an inert atmosphere of argon was added anhydrous oxalic acid (28.8 g, 0.32 mol, 1.0 equiv) and p-Toluenesulfonic acid monohydrate (1.0 g, 0.01 equiv) in order at room temperature. The mixture was stirred at reflux for 3 h. The hot mixture was allowed to cool to room temperature and filtered. The filter cake was washed with 50 mL of acetonitrile and the combined filtrate was evaporated under reduced pressure. The residue was dissolved in 100 mL of ethyl acetate and evaporated to dryness. 50.1 g (70%) 2 was obtained as a white solid. MS m/z [M+H]⁺ (ESI): 119. It was used at next step without further purification.

To a mixture solution of 2 (66.5 g, 0.56 mol, 1.0 equiv) in 1200 mL of dichloromethane and 135 mL of anhydrous pyridine with an inert atmosphere of argon was added benzoyl chloride (72.0 mL, 0.62 mol, 1.1 equiv) dropwise at 0 °C. The resulting solution was stirred for 30 min at 0 °C. The reaction mixture was quenched with 1 N HCl, and washed with 3×200 mL of saturated aqueous sodium chloride. The organic layer was added 2 volume equivalents of hexane over 1 h and stirred for 16 h at 0 °C. The desired product was collected by filtration and dried under vacuum. 75.1 g (60%) 3 was obtained as a white solid. MS m/z [M+H]⁺ (ESI): 223. ¹H NMR (400 MHz, DMSO-d₆): δ 8.08-7.98 (m, 2H), 7.78-7.67 (m, 1H), 7.59-7.51 (m, 2H), 6.12 (d, J=17.9 Hz, 1H), 5.73 (d, J=7.9 Hz, 1H), 4.74-4.71 (m, 1H), 4.55-4.44 (m, 1H), 4.09-4.03 (m, 1H).

To a solution of 3 (16.5 g, 74.3 mmol, 1.0 equiv) in 160 mL of dichloromethane with an inert atmosphere of argon was added 4-dimethylaminopyridine (60 mg, 6.0 mmol, 0.01 equiv), imidazole (10.2 g, 150 mmol, 2.0 equiv) and tert-butyldiphenylchlorosilane (21.4 g, 75.0 mmol, 1.05 equiv) in order at 0 °C. The reaction mixture was warmed to room temperature and stirred overnight. The resulting solution was evaporated under reduced pressure. The residue was dissolved in 300 mL of hexane, washed with 1×100 mL of 1 N HCl, 3×100 mL of water and 3×100 mL of saturated aqueous sodium chloride respectively. The organic phase was dried over anhydrous sodium sulfate, filtered and concentrated under reduced pressure. It was used at next step without further purification. MS m/z [M+H]⁺ (ESI): 461.

To a solution of crude 4 (35.0 g, 76 mmol, 1.0 equiv) in 150 mL of 1,2-dimethoxyethane with an inert atmosphere of argon was added diisobutylaluminium hydride (100 mL, 100 mmol, 1 M solution of in toluene) dropwise over 10 min −78 °C. The mixture solution was stirred for 30 min at −78 °C. The reaction was monitored by TLC and used at next step without further purification.

To a solution of 5 with an inert atmosphere of argon was added a premade solution containing acetic anhydride (35 mL, 367 mmol, 5.0 equiv) and 4-dimethylaminopyridine (14.0 g, 115 mmol, 1.5 equiv) in 40 mL of dichloromethane dropwise at −78 °C. After stirred for 10 min, the reaction mixture was warmed to room temperature and stirred for 2 h. The mixture solution was diluted with 200 mL of hexane and poured into 200 mL of cold 1 N aq. HCl solution. The organic layer was washed with 3×100 mL of water, 3×100 mL of saturated aqueous sodium bicarbonate aqueous and 3×100 mL of saturated aqueous sodium chloride respectively. The organic layer was dried over anhydrous sodium sulfate, filtered and concentrated under reduced pressure. The crude was purified by Flash-Prep-HPLC with the following conditions: Column, C18 silica gel; mobile phase, water (containing 0.1% FA) and acetonitrile (20% acetonitrile up to 100% in 10 min and 100% acetonitrile hold 8 min), UV 254 nm. The fractions were diluted with dichloromethane and dried over anhydrous sodium sulfate. The solid was filtered out and the filtrate was concentrated under reduced pressure. 21.1 g (56% over three steps) of 6 was obtained as a colorless oil. MS m/z [M+H]⁺ (ESI): 505. ¹H NMR (300 MHz, DMSO-d₆): δ 7.89-7.82 (m, 2H), 7.71-7.62 (m, 5H), 7.57-7.28 (m, 8H), 6.44-6.09 (m, 1H), 5.48-5.25 (m, 1H), 4.76-4.59 (m, 1H), 4.19-3.83 (m, 2H), 2.14 (s, 2H), 1.86 (s, 1H), 1.04 (d, J=15.2 Hz, 9H).

To a solution of 6 (10.0 g 59.4 mmol, 1.0 equiv) in 50 mL of anhydrous acetonitrile with an inert atmosphere of argon was added uracil (3.3 g, 29.7 mmol, 1.5 equiv) and N, O-bis(trimethylsilyl)acetamide (8.1 g, 39.6 mmol, 2.0 equiv) in order at room temperature. The mixture was stirred for 30 min at 60 °C. Then trimethylsilyl trifluoromethanesulfonate (7.0 g, 31.7 mmol, 1.5 equiv) was added dropwise and stirred for another 2 h at 60 °C. The reaction mixture was cooled to room temperature, diluted with 200 mL of ethyl acetate and poured into 100 mL of cold saturated aqueous sodium bicarbonate. The organic layer was washed with 3×100 mL of water and 3×100 mL of saturated aqueous sodium chloride respectively. The organic layer was dried over anhydrous sodium sulfate, filtrated and concentrated under reduced pressure. The crude product was used at next step directly without further purification. MS m/z [M+H]⁺ (ESI): 557.

To a solution of triethylamine trihydrofluoride (28.9 g, 17.9 mmol, 10.0 equiv) in 50 mL of dry tetrahydrofuran with an inert atmosphere of argon was added triethylamine (36.4 g, 35.9 mmol, 20.0 equiv) at room temperature. The resulting solution was stirred at room temperature for 10 min. A solution of 7 (10.0 g, 53.9 mmol, 1.0 equiv) in 50 mL of dry tetrahydrofuran was added to the resulting solution. The reaction mixture was stirred for overnight at room temperature and diluted with 300 mL of dichloromethane, washed with 2×100 mL of saturated aqueous sodium bicarbonate and 2×100 mL of saturated aqueous sodium chloride respectively. The organic layer was dried over anhydrous sodium sulfate, filtered and concentrated under reduced pressure. The residue was purified by Flash-Prep-HPLC with the following conditions: Column, C18 silica gel; mobile phase, water (containing 0.1% FA) and acetonitrile (10% acetonitrile up to 100% in 12 min), UV 254 nm. The fractions were diluted with dichloromethane, dried over anhydrous sodium sulfate and concentrated under reduced pressure. 5.0 g (80% over two steps) of 8 was obtained as a light yellow solid. MS m/z [M+H]⁺ (ESI): 319. ¹H NMR (400 MHz, DMSO-d₆): δ 11.35 (d, J=2.2 Hz, 1H), 8.05-7.94 (m, 2H), 7.76 (d, J=8.2 Hz, 1H), 7.73-7.65 (m, 1H), 7.54-7.32 (m, 2H), 5.92-5.75 (m, 2H), 5.67-5.46 (m, 1H), 5.26 (d, J=1.8 Hz, 1H), 4.36-4.25 (m, 1H), 4.14-3.89 (m, 2H).

To a solution of 8 (10.0 g, 31.4 mmol, 1.0 equiv) in 80 mL of dry dichloromethane with an inert atmosphere of argon was added 2,4,6-collidine (22.8 g, 188.1 mmol, 6.0 equiv) and 4,4′-dimethoxytrityl chloride (26.6 g, 78.7 mmol, 2.5 equiv) in order at room temperature. The resulting solution was stirred for 24 h at room temperature. The reaction mixture was quenched by 10 mL of methanol, diluted with 500 mL of dichloromethane, washed with 2×200 mL of saturated aqueous sodium bicarbonate and 2×200 mL of saturated aqueous sodium chloride respectively. The organic layer was dried over anhydrous sodium sulfate, filtered and concentrated under reduced pressure. The residue was dissolved in 400 mL of ammonia (7M NH₃ in methanol) and stirred for 24 h at room temperature with an inert atmosphere of argon. The reaction mixture was concentrated under reduced pressure. The residue was purified by Flash-Prep-HPLC with the following conditions: Column, C18 silica gel; mobile phase, water (containing 0.04% NH₄HCO₃) and acetonitrile (15% acetonitrile up to 100% in 10 min and 100% acetonitrile hold 6 min), UV 254 nm. The fractions were diluted with dichloromethane, dried over anhydrous sodium sulfate and concentrated under reduced pressure. 12.1 g (75%) of 81 was obtained as a light yellow oil. MS m/z [M−H]⁻ (ESI): 515.

To a solution of 81 (10.0 g, 19.4 mmol, 1.0 equiv) in 100 mL of dry N,N-dimethylformamide with an inert atmosphere of argon was added imidazole (3.9 g, 57.3 mmol, 3.0 equiv) and tert-Butyldimethylsilyl chloride (8.8 g, 58.1 mmol, 3.0 equiv) in order at room temperature. The resulting solution was stirred for 12 h at room temperature. The reaction mixture was diluted with 500 mL of dichloromethane, washed with 2×200 mL of saturated aqueous sodium bicarbonate and 1×200 mL of saturated aqueous sodium chloride respectively. The organic phase was dried over anhydrous sodium sulfate, filtered and concentrated under reduced pressure. The residue was dissolved in 120 mL of dichloromethane, added trifluoroacetic acid (21.7 g, 190.5 mmol, 10.0 equiv) and triethylsilane (11.1 g, 95.2 mmol, 5.0 equiv) in order at room temperature and stirred for 1 h with an inert atmosphere of argon at room temperature. The reaction mixture was diluted with 400 mL of dichloromethane, washed with 2×150 mL of saturated aqueous sodium bicarbonate and 1×150 mL of saturated aqueous sodium chloride respectively. The organic phase was dried over anhydrous sodium sulfate, filtered and concentrated under reduced pressure. The residue was purified by Flash-Prep-HPLC with the following conditions: Column, C18 silica gel; mobile phase, water (containing 0.1% FA) and acetonitrile (15% acetonitrile up to 100% in 10 min and 100% acetonitrile hold 6 min), UV 254 nm. The fractions were diluted with dichloromethane, dried over anhydrous sodium sulfate and concentrated under reduced pressure. 4.7 g (75%) of 82 was obtained as a light yellow solid. MS m/z [M−H]⁻ (ESI): 515. ¹H NMR (300 MHz, DMSO-d₆): δ 11.29 (s, 1H), 7.62 (d, J=8.2 Hz, 1H), 5.58-5.55 (m, 2H), 5.43 (d, J=2.6 Hz, 1H), 4.17-3.94 (m, 4H), 0.86 (s, 9H), 0.10 (d, J=4.0 Hz, 6H).

To a solution of 82 (4.0 g, 11.9 mmol, 1.0 equiv) in 40 mL of dry tetrahydrofuran with an inert atmosphere of argon was added (diethoxyphosphoryl)methyl 4-methylbenzenesulfonate (7.7 g, 23.9 mmol, 2.0 equiv) at room temperature. The reaction mixture was added sodium hydride (1.9 g, 47.7 mmol, 4.0 equiv) at 0 °C and stirred for 3 h at the same temperature with an inert atmosphere of argon. The reaction was quenched with 6 mL of acetic acid (1M) and diluted with 150 mL of ethyl acetate, washed with 1×50 mL of H₂O and 1×50 mL of saturated aqueous sodium chloride respectively. The organic phase was dried over anhydrous sodium sulfate, filtered and concentrated under reduced pressure. The residue was applied onto a silica gel column with ethyl acetate/petroleum ether (1:10-5:1). The fractions of desired product were concentrated under reduced pressure. 2.5 g (60%) of 53 was obtained as a light yellow oil. MS m/z [M+H]⁺ (ESI): 479. ¹H NMR (400 MHz, DMSO-d₆): δ11.32 (s, 1H), 7.53 (d, J=8.1 Hz, 1H), 5.60 (d, J=1.1 Hz, 1H), 5.51 (d, J=8.1 Hz, 1H), 4.35-4.28 (m, 2H), 4.07-3.84 (m, 8H), 1.24-1.20 (m, 6H), 0.87 (s, 9H), 0.13 (d, J=2.5 Hz, 6H). ³¹P NMR (162 MHz, DMSO): δ 20.74.

A mixture solution of 53 (3.2 g, 6.7 mmol, 1.0 equiv) in 32 mL of formic acid and H₂O (v/v=1:1) with an inert atmosphere of argon was stirred for 24 h at room temperature. The resulting solution was concentrated under vacuum. The crude product was diluted with 300 mL of ethyl acetate, washed 2×50 mL of water and 1×50 mL of saturated aqueous sodium bicarbonate respectively. The organic phase was dried over anhydrous sodium sulfate, filtered and concentrated under vacuum. The residue was purified by Flash-Prep-HPLC with the following conditions: Column, C18 silica gel; mobile phase, water and acetonitrile (15% acetonitrile up to 35% in 20 min), UV 254 nm. The fractions were diluted with dichloromethane, dried over anhydrous sodium sulfate and concentrated under reduced pressure. 1.9 g (80%) of 14 was obtained as a light yellow oil. MS m/z [M+H]⁺ (ESI): 365. ¹H NMR (300 MHz, DMSO-d₆): δ 11.31 (s, 1H), 7.55 (d, J=8.1 Hz, 1H), 5.94 (d, J=4.1 Hz, 1H), 5.68 (d, J=1.4 Hz, 1H), 5.53 (d, J=8.1 Hz, 1H), 4.28 (d, J=10.2 Hz, 1H), 4.17 (s, 1H), 4.09-3.97 (m, 6H), 3.95-3.84 (m, 2H), 1.23-1.09 (m, 6H). ³¹P NMR (121 MHz, DMSO): δ20.84.

To a solution of 14 (2.2 g, 6.0 mmol, 1.0 equiv) in 20 mL of dichloromethane with an inert atmosphere of argon was added 2-Cyanoethyl N,N,N′,N′-tetraisopropylphosphorodiamidite (2.4 g, 7.8 mmol, 1.3 equiv) and 4,5-Dicyanoimidazole (785 mg, 6.6 mmol, 1.1 equiv) in order at room temperature. The reaction mixture was stirred for 1 h at room temperature. The reacting solution was diluted with 200 mL of dichloromethane, washed with 2×100 mL of saturated aqueous sodium bicarbonate and 1×100 mL of saturated aqueous sodium chloride respectively. The organic phase was dried over anhydrous sodium sulfate, filtered and concentrated till no residual solvent left under reduced pressure. The residue was purified by Flash-Prep-HPLC with the following conditions: Column, C18 silica gel; mobile phase, water (containing 0.04% NH₄HCO₃) and acetonitrile (20% acetonitrile up to 100% in 10 min and 100% acetonitrile hold 5 min). The fraction was diluted with 500 mL of dichloromethane. The organic phase was dried over anhydrous sodium sulfate, filtered and concentrated under reduced pressure. 2.2 g (70%) 100 was obtained as a light yellow oil. MS m/z [M+H]⁺ (ESI):565. ¹H NMR (300 MHz, Acetonitrile-d₃): δ 9.05 (s, 1H), 7.57-7.54 (m, 1H), 5.88-5.83 (m, 1H), 5.57-5.47 (m, 1H), 4.55-4.41 (m, 1H), 4.39-4.29 (m, 1H), 4.23-4.00 (m, 6H), 3.95-3.76 (m, 4H), 3.73-3.57 (m, 2H), 2.75-2.65 (m, 2H), 1.31-1.27 (m, 6H), 1.22-1.19 (m, 12H). ³¹P NMR (121 MHz, CD₃CN): δ 151.05, 150.14, 19.82, 19.71.

### Example 2-5

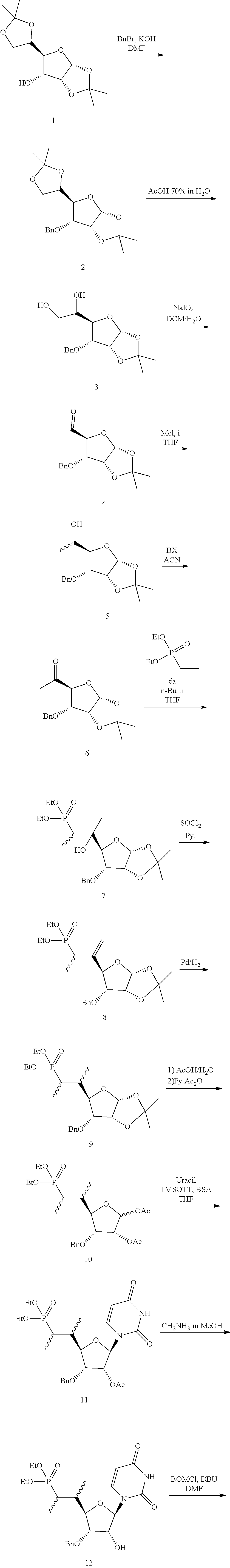

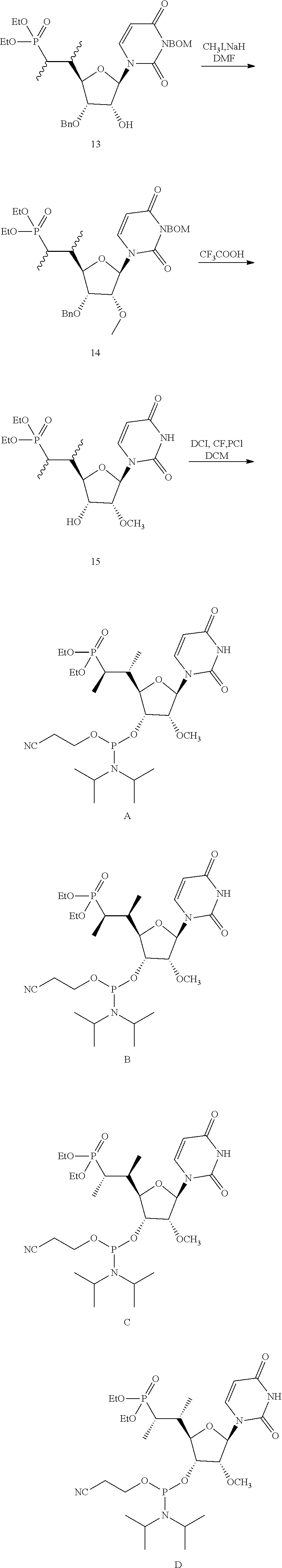

To a solution of 1(120.0 g, 461.5 mmol) and KOH (31.0 g, 553.6 mmol) in DMF (500.0 mL) was added BnBr (118.0 g, 690.1 mmol) at 0 °C for 30 min, then it was stirred at r.t. overnight. LC-MS and TLC monitored 1 was consumed completely. The reaction was dissolved with water, and the pH was adjusted to pH=9 by progressively adding solid NaHCO₃, then it was extracted by EA (700.0 mL*3). The organic layer was washed with water (1.0 L*2) and NaCl anhydrous, dried over anhydrous Na₂SO₄ and evaporated in the vacuo to give crude product 2 (150.0 g, crude) as white oil. ESI-MS: m/z 334.1.

Crude product 2 (150.0 g, crude) was dissolved in 70% AcOH in H₂O (700.0 ml) at r.t., then it was stirred at room temperature overnight. TLC showed 2 was consumed completely. The reaction was quenched with solid NaHCO₃ aqueous, and it was extracted by EA (700.0 mL*3). The organic layer was washed with water (1.0 L*2) and NaCl aqueous, dried over anhydrous Na₂SO₄ and evaporated in the vacuo to give crude product which was purified by column chromatography (SiO₂, PE/EA=2:1 to 0:1) to give 3 (110.0 g, 90.5% purity, 76.8% yield) as a light yellow solid. ESI-MS: m/z 333.1; ¹HNMR (400 MHz, CDCl₃): δ 7.36 (m, 5H), 5.76 (d, J=3.72 Hz, 1H), 4.78 (d, J=11.28 Hz, 1H), 4.60 (t, J=4.04 Hz, 1H), 4.56 (d, J=11.28 Hz, 1H), 4.11 (m, 1H), 4.00 (m, 1H), 3.93 (m, 1H), 3.67 (m, 2H), 2.55 (s, 2H), 1.59 (s, 3H), 1.36 (s, 3H).

To a stirred solution of 3 (110. g, 350.0 mmol) in DCM (1.2 L) was added the solution of NaIO₄ (153.0 g, 710 mmol) in H₂O (600.0 mL) at r.t., then it was stirred for 1.0 h at room temperature. TLC monitored it worked completed. The reaction was dissolved by water, and it was extracted by DCM (700.0 mL*3). The organic layer was washed with water (1.0 L*2) and NaCl aqueous, dried over anhydrous Na₂SO₄ and concentrated under reduced pressure to give 4 (92.0 g, crude) as a light yellow oil. ESI-MS: m/z 262.1; ¹HNMR (400 MHz, CDCl₃): δ 9.60 (d, J=1.76 Hz, 1H), 7.35 (m, 5H), 5.81 (d, J=3.44 Hz, 1H), 4.74 (d, J=12.12 Hz, 1H), 4.60 (m, 2H), 4.48(dd, J=1.68 Hz and 9.24 Hz, 1H), 3.85 (q, J=4.28 Hz 1H), 1.60 (s, 3H), 1.37 (s, 3H).

To a solution of 4 (85.0 g, crude) in THF (300.0 mL) was added the solution of MeLi in diethoxymethane (1.6M, 287.0 mL) dropwise at 0 °C for 0.5 h, then the reaction was stirred at 0 °C for 2.5 h. TLC showed 4 was consumed completely. The reaction was quenched by NH₄Cl aqueous, and it was extracted by EA (400.0 mL*3). The organic layer was washed with water (1.0 L*2) and NaCl aqueous, dried over anhydrous Na₂SO₄ and evaporated in the vacuo to give 5 (86.6 g, crude) as a light yellow oil. ESI-MS: m/z 278.1; ¹HNMR (400 MHz, CDCl₃): δ 7.36 (m, 5H), 5.73 (d, J=3.6 Hz, 1H), 4.76 (m, 1H), 4.58 (m, 2H), 4.02 (m, 1H), 3.91 (m, 1H), 3.81 (m, 1H), 1.59 (s, 3H), 1.36 (m, 3H), 1.25 (dd, J=6.6 Hz, 19.2 Hz, 3H).

The mixture of 5 (86.6 g, crude) and 2-Iodoxybenzoic acid (165.0 g, 589.3 mmol) in ACN (1.0 L) was stirred at 80 °C for 2 h, and then TLC showed 5 was consumed completely. Then it was filtered, the organic layer was concentrated to afford crude product which was purified by column chromatography (SiO₂, PE/EA=10:1 to 8:1) to give 6 (48.6 g, 90% purity, 56.5% yield) as yellow solid. ESI-MS: m/z 276.1; ¹HNMR (400 MHz, CDCl₃) δ 7.36 (m, 5H), 5.82 (d, J=3.56 Hz, 1H), 4.77 (d, J=11.96 Hz, 1H), 4.60 (m, 2H), 4.52 (d, J=9.16 Hz, 1H), 3.77 (m, 1H), 2.19 (s, 3H), 1.60 (s, 3H), 1.37 (s, 3H).

To a stirred solution of diethyl ethylphosphonate (20.7 g, 124.7 mmol) in dried THF (600.0 mL) was added n-BuLi (1.6M, 86.0 mL) at −70 °C dropwise, and it was stirred at −70 °C for 0.5 h. 6 (40.0 g, 137.0 mmol) in THF(300.0 mL) was added at −70 °C for 20 min, and it was stirred at −70 °C for 2 h. LCMS showed 6 was consumed completely. It was quenched by NH₄Cl aqueous and extracted by EA (600 mL*3). The organic layer washed with water (600.0 mL*2) and NaCl aqueous, dried by anhydrous Na₂SO₄ and filtered, then the organic layer was concentrated. The crude product was purified by column chromatography (SiO₂, PE/EA=1:1) to give 7 (33.6 g, 73.3 mmol, 93% purity) as yellow solid. ESI-MS: m/z 459.2 [M+H]⁺; ¹HNMR (400 MHz, CDCl₃) δ 7.34 (m, 5H), 5.77 (m, 1H), 4.83-4.71 (m, 1H), 4.64-4.52 (m, 2H), 4.23-3.86 (m, 7H), 1.75 (m, 1H), 1.58 (t, J=7.6 Hz, 3H), 1.30 (m, 15H). 31P-NMR (162 MHz, CDCl₃) δ=33.74, 32.93, 32.19, 31.53.

To a solution of 7 (33.6 g, 73.4 mmol) in pyridine (200.0 mL) was added SOCl₂ (86.5 g, 73.3 mmol) at ice bath for 30 min, it was stirred at r.t. for 30 min. LCMS showed 7 was consumed completely. The mixture was quenched by NaHCO₃ (200.0 mL) aqueous, extracted by EA (400.0 mL*2) and washed by NaCl aqueous. After dried by anhydrous Na₂SO₄ and filtered, the organic layer was concentrated to afford crude product which was purified by column chromatography (SiO₂, PE/EA=1:1) to give 8 (25.0 g, 56.8 mmol) as a light yellow solid. ESI-MS: m/z 441.2 [M+H]⁺; ¹HNMR (400 MHz, CDCl₃) δ 7.34 (m, 5H), 5.78 (t, J=3.32 Hz, 1H), 5.43 (m, 1H), 5.31 (m, J=5.36 Hz 1H), 4.75 (m, 1H), 4.56 (m, 3H), 4.07 (m, 4H), 3.69 (m, 1H), 2.81 (m, 1H), 1.60 (s, 3H), 1.30 (m, 12H); ³¹P-NMR (162 MHz, CDCl₃) δ=30.33, 29.97.

To a solution of 8 (25.5 g, 56.0 mmol) in CH₃OH (200.0 mL) was added Pd/C (3.8 g, 15%) at r.t., it was stirred at r.t. for 30 min under the atmosphere of N₂, then it was filtered. Another part of Pd/C (3.8 g, 15%) was added, and it was stirred at 50 °C for 3 h. LCMS showed 8 was consumed completely. After filtered, the organic layer was concentrated to afford crude product which was purified by column chromatography to afford 9 (15.0 g, 34.0 mmol, 58.6% yield) as a light yellow oil. ESI-MS: m/z 443.5 [M+H]⁺; ¹HNMR (400 MHz, CDCl₃) δ 7.35 (m, 5H), 5.69 (m, 1H), 4.81-4.73 (m, 1H), 4.60-4.49 (m, 2H), 4.14-4.01 (m, 5H), 3.56-3.48 (m, 1H), 2.47-2.10 (m, 2H), 1.58 (m, 3H), 1.37-1.23 (m, 15H). ³¹P-NMR (162 MHz, CDCl₃) δ=34.76, 34.17, 33.95, 33.55.

To a solution of 9 (15.0 g, 34.0 mmol) in 60% AcOH/H₂O (75.0 mL) was stirred at 100 °C for 3 h. TLC showed 9 was consumed completely, then it was concentrated in vacuum to give crude intermediate. The crude intermediate and DMAP (828.0 mg, 6.8 mmol) was dissolved in dry pyridine (60.0 mL), then Ac₂O (20.8 g, 203.9 mmol) was added at 0 °C for 10 min. It was stirred at r.t. for 2 h, then LCMS monitored it was completed. The mixture was quenched by NaHCO₃ (100.0 mL) aqueous and extracted by EA (100.0 mL*2). The organic layer was washed by NaCl aqueous and dried by anhydrous Na₂SO₄. After filtered, the organic layer was concentrated to afford crude product which was purified by column chromatography to afford 10 (13.8 g, 28.4 mmol, 83.7% yield) as yellow oil. ESI-MS: m/z 509.2 [M+Na]⁺; ¹HNMR (400 MHz, CDCl₃) δ 7.36-7.23 (m, 5H), 6.36-6.07 (m, 1H), 5.36-5.04 (m, 1H), 4.65-4.52 (m, 1H), 4.49-4.42 (m, 1H), 4.34-4.84 (m, 6H), 2.45-2.18 (m, 1H), 2.13-2.04 (m, 6H), 1.95-1.69 (m, 1H), 1.35-0.95 (m, 12H). ³¹PNMR (162 MHz, CDCl₃) δ=34.43, 34.17, 34.15, 33.56, 33.44, 33.39, 32.61.

A solution of uracil (4.1 g, 36.3 mmol) and BSA (15.9 g, 77.7 mmol) in dry ACN (80.0 mL) was stirred at 50 °C until the mixture to be clear. Then the solution of 10 (11.8 g, 24.3 mmol) in dry ACN (40.0 mL) was added and TMSOTf (5.5 g, 24.8 mmol) added dropwise at r.t. for 30 min. The mixture was stirred at r.t. for 5 h. LMCS showed 10 was consumed completely. The mixture was quenched by NaHCO₃ (100.0 mL) aqueous and extracted by EA (200.0 mL*2). The organic layer was washed by NaCl aqueous and dried by anhydrous Na₂SO₄. After filtered, the organic layer was concentrated to afford crude product which was purified by column chromatography (SiO₂, PE:EA=1:2) to afford 11 (10.3 g, 19.1 mmol, 78.8% yield) as yellow solid. ESI-MS: m/z 539.1 [M+H]⁺; ¹HNMR (400 MHz, CDCl₃) δ 9.94-9.86 (m, 1H), 7.37-7.27 (m, 6H), 5.77-5.71 (m, 2H), 5.45-5.29 (m, 1H), 4.60-4.46 (m, 2H), 4.32-4.26 (m, 1H), 4.14-4.03 (m, 5H), 2.38-2.34 (m, 1H), 2.22-2.16 (m, 1H), 2.11-2.08 (m, 3H), 1.38-1.02 (m, 12H). ³¹PNMR (162 MHz, CDCl₃) δ=34.16, 33.56, 33.29, 33.03, 32.44.

The solution of 11 (10.3 g, 19.1 mmol) in CH₃NH₂ (100.0 mL, 30% in MeOH) was stirred at r.t. for 1 h. LCMS showed 11 was consumed completely. Then the mixture was concentrated in vacuum to give 12 (11.0 g, crude). ESI-MS: m/z 497.2 [M+H]⁺; ³¹PNMR (162 MHz, CDCl₃) δ=34.18, 33.17, 33.02, 33.03, 32.40.

To a stirred solution of 12 (9.1 g, crude) and DBU (8.4 g, 55.2 mmol) in dry DMF (50.0 mL) was added Benzylchloromethyl ether (5.7 g, 36.4 mmol) at 0 °C for 10 min. Then the mixture was stirred at r.t. overnight. LCMS showed 12 was consumed completely. Then the mixture was quenched by NaHCO₃ (50.0 mL) aqueous and extracted by EA (100.0 mL*2). The organic layer was washed by NaCl aqueous and dried by anhydrous Na₂SO₄. After filtered, the organic layer was concentrated to afford crude product which product was purified by column chromatography (SiO₂, PE:EA=1:1) to give 13 (7.0 g, 11.4 mmol, 59.4% yield). ESI-MS: m/z 617.4 [M+H]⁺; ¹H NMR (400 MHz, CDCl₃) δ 9.94-9.86 (m, 1H), 7.38-7.17 (m, 11H), 5.83-5.74 (m, 2H), 5.66-5.43 (m, 3H), 4.78-4.55 (m, 4H), 4.37-4.29 (m, 1H), 4.22-3.93 (m, 6H), 3.10-2.99 (m, 1H), 2.45-2.11 (m, 1H), 2.04-1.93 (m, 3H), 1.65 (m, 1H), 1.34-1.03 (m, 12H). ³¹PNMR (162 MHz, CDCl₃) δ=34.15, 33.17, 33.03, 33.46.

The solution of 13 (7.0 g, 10.2 mmol), NaI (767.0 mg, 5.1 mmol) and Ag₂O (3.6 g, 15.3 mmol) in Mel (30.0 mL) was stirred at 40 °C for 1 h, LCMS showed 13 was consumed completely. After filtered, the organic layer was concentrated to afford crude product which was purified by column chromatography (SiO₂, PE:EA=1:1) to afford 14-P1 (3.9 g, 6.2 mmol) and 14-P2 (3.1 g, 4.9 mmol) as yellow solid. ESI-MS: m/z 631.3 [M+H]⁺; 14-P1: 31PNMR (162 MHz, CDCl₃) δ=33.03, 32.85; 14-P2: ³¹PNMR (162 MHz, CDCl₃) δ=34.27, 33.55.

The solution of 14-P1 (3.9 g, 6.2 mmol) in CF₃COOH (20.0 mL) was stirred at 80 °C for 1 h, LCMS showed 14 was consumed completely. After concentrated, the crude product was purified by column chromatography (SiO₂, PE:EA=0:1) to give the mixture product (2.5 g, 93% purity, 96.2% yield). The mixture was separated by SFC to give isomer 1 of 15 (500.0 mg, 1.2 mmol, 20.0% yield) and isomer 2 of 15 (760 mg, 1.8 mmol, 30.4% yield) as white solid. The solution of 14-P2 (3.1 g, 4.9 mmol) in CF₃COOH (20.0 mL) was stirred at 80 °C for 1 h, LCMS showed 14-P2 was consumed completely. After concentrated, the crude product was purified by column chromatography (SiO₂, PE:EA=0:1) to give the mixture product (1.6 g, 91% purity, 77.4% yield). The mixture was separated by SFC to afford isomer 3 of 15 (130.0 mg, 0.3 mmol, 8.1% yield) and isomer 4 of 15 (800 mg, 1.9 mmol, 50.0% yield) as white solid.

Isomer 1 of 15: ¹HNMR (400 MHz, CDCl₃) δ 8.98 (s, 1H), 7.33 (d, J=8.12 Hz, 1H), 5.76 (m, 2H), 4.24 (t, J=7.9 Hz, 1H), 4.11 (m, 5H), 3.89 (d, J=5.6 Hz, 1H), 3.59 (s, 3H), 2.38-2.27 (m, 1H), 2.15-2.04 (m, 1H), 1.35-1.18 (m, 12H). ³¹PNMR (162 MHz, CDCl₃) δ=33.29; ESI-MS: m/z 421.2[M+H]⁺

Isomer 2 of 15: ¹HNMR (400 MHz, CDCl₃) δ 8.69 (s, 1H), 7.40 (d, J=8.1 Hz, 1H), 5.72-5.69 (m, 2H), 4.04-3.97 (m, 5H), 3.83 (q, J=6.2 Hz, 1H), 3.72 (m, 1H), 3.52 (s, 3H), 2.27-2.16 (m, 1H), 2.03-1.88 (m, 1H), 1.26-1.17 (m, 12H). ³¹PNMR (162 MHz, CDCl₃) δ=33.56; ESI-MS: m/z 421.2[M+H]⁺

Isomer 3 of 15: ¹HNMR (400 MHz, CDCl₃) δ 8.69 (s, 1H), 7.40 (d, J=8.1 Hz, 1H), 5.83-5.76 (m, 2H), 4.20-4.05 (m, 4H), 3.91-3.88 (m, 1H), 3.74-3.71 (m, 2H), 3.61 (s, 3H), 2.46-2.31 (m, 2H), 1.35-1.32 (t, J=7.04 Hz, 6H), 1.19-1.12 (m, 6H). ³¹PNMR (162 MHz, CDCl₃) δ=33.51; ESI-MS: m/z 421.2[M+H]⁺

Isomer 4 of 15: ¹HNMR (400 MHz, CDCl₃) δ 8.89 (s, 1H), 7.33 (d, J=8.04 Hz, 1H), 5.82-5.70 (m, 2H), 4.11-4.02 (m, 4H), 3.88 (m, 1H), 3.72 (s, 1H), 3.63 (t, J=6.2 Hz, 9.28 Hz, 1H), 3.52 (s, 3H), 2.38-2.23 (m, 2H), 1.27-1.01 (m, 12H). ³¹PNMR (162 MHz, CDCl₃) δ=34.18; ESI-MS: m/z 421.1 [M+H]⁺ Synthesis of A

To a stirred solution of isomer 1 of 15 (500 mg, 1.2 mmol) and DCI (126.4 mg, 1.1 mmol) in dry DCM (5.0 mL) was added CEP[N(iPr)₂]₂ (430.0 mg, 1.4 mmol) under N₂ atmosphere. The mixture was stirred at 30 °C for 1 h. LCMS showed isomer 1 of 15 was consumed completely. Then the solution was diluted with DCM (10 mL) and washed with H₂O (10 mL*3). After dried by anhydrous Na₂SO₄, the organic layer was concentrated under reduced pressure and the residue was purified by Flash-Prep-HPLC with the following conditions (IntelHash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=6/1; Detector, UV 254 nm. This resulted in to give A (500 mg, 0.8 mmol, 95% purity, 65% yield) as a white solid. ESI-MS: m/z 619.2[M−H]⁻; ¹HNMR (400 MHz, CDCl₃) δ 9.10 (s, 1H), 7.30 (m, J=8.4 Hz, 1H), 5.82 (t, J=4.4 Hz, 1H), 5.76 (d, J=8.4 Hz, 1H), 4.34 (m, 1H), 4.10 (m, 5H), 3.84 (m, 5H), 3.48 (m, 5H), 3.50 (m, 3H), 2.72-2.66 (m, 2H), 2.20 (m, 1H), 2.04-1.97 (m, 1H), 1.34-1.17 (m, 24H); ³¹P NMR (162 MHz, CDCl₃) δ=149.59, 33.43, 34.03.

### Synthesis of B

To a stirred solution of isomer 2 of 15 (760 mg, 1.8 mmol) and DCI (192.2 mg, 1.6 mmol) in dry DCM (5.0 mL) was added CEP[N(iPr)₂]₂(653.6 mg, 2.1 mmol) under N₂ atmosphere. The mixture was stirred at 30 °C for 1 h. LCMS showed isomer 2 of 15 was consumed completely. Then the solution was diluted with DCM (10 mL) and washed with H₂O (10 mL*3). After dried by anhydrous Na₂SO₄, the organic layer was concentrated under reduced pressure and the residue was purified by Flash-Prep-HPLC with the following conditions (IntelHash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=6/1; Detector, UV 254 nm. This resulted in to give B (500 mg, 0.8 mmol, 95% purity, 45% yield) as a white solid. ESI-MS: m/z 619.2 [M−H]⁻; ¹HNMR (400 MHz, CDCl₃) δ 9.40 (s, 1H), 7.45 (m, 1H), 5.77 (m, 2H), 4.27-3.60 (m, 11H), 3.49 (d, J=14 Hz, 3H), 2.66 (m, 2H), 2.38-2.30 (m, 1H), 2.01 (m, 1H), 1.33-1.15 (m, 24H). ³¹PNMR (162 MHz, CDCl₃) δ=149.35, 32.76, 32.73.

### Synthesis of C

To a stirred solution of isomer 3 of 15 (130 mg, 0.3 mmol) and DCI (32.9 mg, 0.3 mmol) in dry DCM (2.0 mL) was added CEP[N(iPr)2]2 (111.8 mg, 0.4 mmol) under N₂ atmosphere. The mixture was stirred at 30 °C for 1 h. LCMS showed isomer 3 of 15 was consumed completely. Then the solution was diluted with DCM (10 mL) and washed with H₂O (10 mL*3). After dried by anhydrous Na₂SO₄, the organic layer was concentrated under reduced pressure and the residue was purified by Flash-Prep-HPLC with the following conditions (IntelHash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=6/1; Detector, UV 254 nm. This resulted in to give C (110 mg, 0.2 mmol, 95% purity, 57% yield) as a white solid. ESI-MS: m/z 619.2[M−H]⁻; ¹HNMR (400 MHz, CDCl₃) δ 8.50 (s, 1H), 7.31 (m, 1H), 5.84 (m, 1H), 5.75 (d, J=8.4 Hz, 1H), 4.16-3.59 (m, 11H), 2.70 (m, 2H), 2.40-2.18 (m, 2H), 1.41-1.12 (m, 24H). ³¹PNMR (162 MHz, CDCl₃) δ=150.21, 149.66, 33.61, 33.01.

### Synthesis of D

To a stirred solution of isomer 4 of 15 (800 mg, 1.9 mmol) and DCI (202.3 mg, 1.7 mmol) in dry DCM (5.5 mL) was added CEP[N(iPr)2]2 (688.0 mg, 2.3 mmol) under N₂ atmosphere. The mixture was stirred at 30 °C for 1 h. LCMS showed isomer 4 of 15 was consumed completely. Then the solution was diluted with DCM (10 mL) and washed with H₂O (10 mL*3). After dried by anhydrous Na₂SO₄, the organic layer was concentrated under reduced pressure and the residue was purified by Flash-Prep-HPLC with the following conditions (IntelHash-1): Column, C18 silica gel; mobile phase, CH₃CN/H₂O (0.5% NH₄HCO₃)=1/1 increasing to CH₃CN/H₂O (0.5% NH₄HCO₃)=1/0 within 20 min, the eluted product was collected at CH₃CN/H₂O (0.5% NH₄HCO₃)=6/1; Detector, UV 254 nm. This resulted in to give D (760 mg, 1.23 mmol, 95% purity, 64% yield) as a white solid. ESI-MS: m/z 619.2[M−H]⁻; ¹HNMR (400 MHz, CDCl₃) δ 9.40 (s, 1H), 7.45 (m, 1H), 6.03 (t, J=4.8 Hz, 1H), 5.70 (m, 1H), 4.22-4.06 (m, 5H), 3.94-3.61 (m, 6H), 3.48 (d, J=10.8 Hz, 3H), 2.64 (m, 2H), 2.49-2.29 (m, 2H), 1.39-1.29 (m, 6H), 1.24-1.15 (m, 12H), 1.12-1.04 (m, 6H). ³¹P NMR (162 MHz, CDCl₃) δ=150.3, 149.90, 34.52, 34.36.

### Example 3: Exemplary Synthesis of Oxetane VP

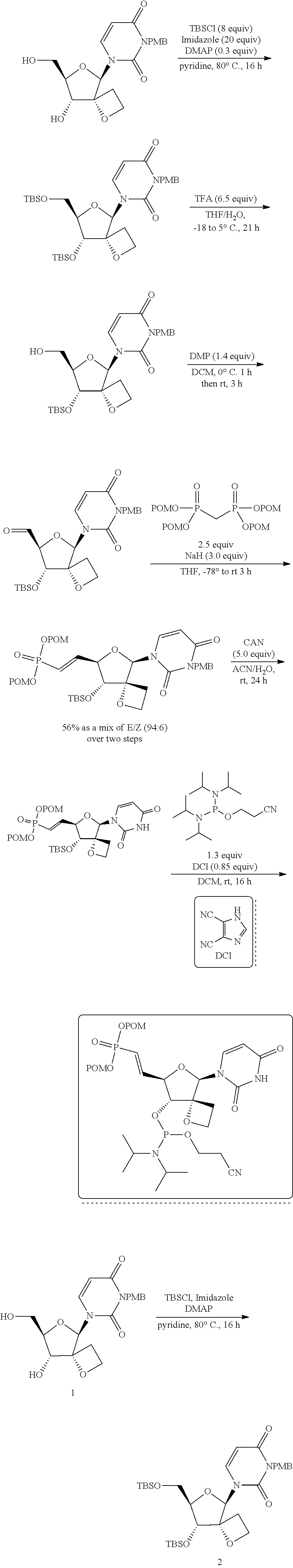

Under a N₂ atmosphere 1 (Prepared according to: T. Jonckers et al. J. Med. Chem. 2016, 59, 5790-5798) (10.0 g, 25.6 mmol), imidazole (34.9 g, 512 mmol) and DMAP (0.94 g, 7.7 mmol) were dissolved in DMF (100 ml). TBSCl (30.9 g, 205 mmol) was added portion wise at 0 °C. After complete addition the mixture was stirred at 80 °C for 16 h. To the reaction was added sat. aq. NH₄Cl solution and the aqueous layer was extracted with EtOAc (3×). The combined organic layers were washed with aq. 5% LiCl solution (2×), dried over Na₂SO₄ and concentrated in vacuo affording 2 (21.1 g) as a crude product, which was used in the next step without further purification.

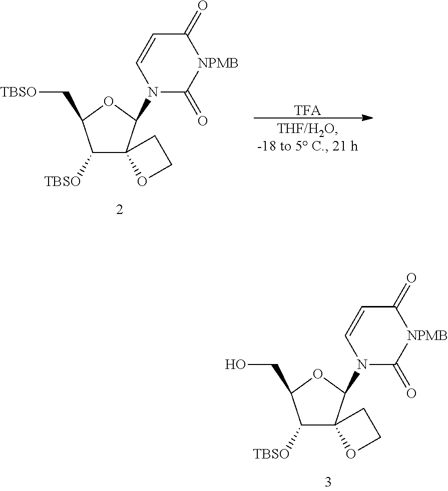

2 (15.8 g, 25.6 mmol) was dissolved in THF (51.7 ml) and distilled water (13.7 ml). The mixture was cooled to 0 °C and TFA (13.7 ml) was added dropwise over 15 min while the internal reaction temperature was maintained below 2 °C. The mixture was stirred at 0 °C for 1 h, then at −18 °C for 14 h. The reaction was warmed to 0 °C and stirring was continued for 4 h, followed by 4 h at 5 °C. The reaction was basified with NH₄OH solution to pH ~8, diluted with water and the aqueous layer was extracted with EtOAc (3×). The combined organic layers were dried over Na₂SO₄ and concentrated in vacuo. The crude was purified by flash silica gel chromatography (DCM/MeOH, gradient from 100:0 to 98:2) affording 3 (7.6 g, 59%).

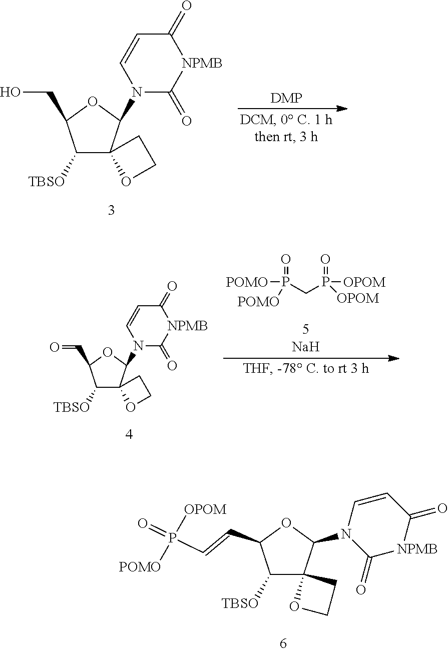

Under a N₂ atmosphere 3 (3.0 g, 6.0 mmol) was dissolved in DCM (100 ml). DMP (3.6 g, 8.4 mmol) was added at 0 °C and the mixture was stirred at 0 °C for 1 h, then at rt for another 3 h. To the mixture was added 50 ml of a mixture of 30% aq. Na₂S₂O₃ solution and sat. aq. NaHCO₃ (1:1) and the aqueous layer was extracted with EtOAc (3×). The combined organic layers were dried over Na₂SO₄ and concentrated in vacuo affording 4 (4.4 g) as a crude product, which was used in the next step without further purification.

Under a N₂ atmosphere NaH (60% dispersion in mineral oil, 720 mg, 18 mmol) was suspended in THF (10 ml) and the mixture was cooled to −78 °C. A solution of 5 (9.5 g, 14.5 mmol) in THF (11 ml) was added and the mixture was stirred at −78° for 20 min. To this mixture a solution of 4 (3.0 g, 6 mmol) in THF (21 ml) was added dropwise over a period of 30 min at −78 °C and stirring was continued for 100 min at the same temperature. The mixture was warmed to 0 °C and stirred for 1 h, followed by another 90 min at rt. The crude reaction mixture was poured into 100 ml sat. aq. NH₄Cl solution, and the product was extracted into 100 ml ethyl acetate (2×). The organic layer was washed with brine, dried over anhydrous Na₂SO₄. The solution was then concentrated under reduced pressure, and the residue was purified by flash silica gel chromatography (heptane:EtOAc, gradient from 100:0 to 0:100) to obtain 6 (2.7 g, 56%) as a 90:10 mixture of E:Z isomer.

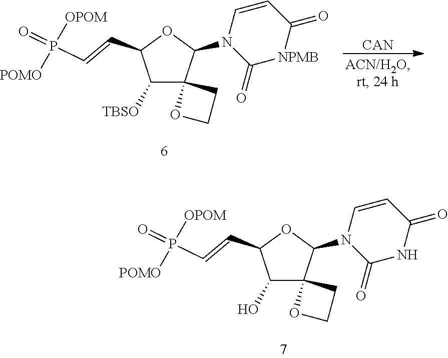

6 (2.7 g, 2.7 mmol) was dissolved in MeCN (26 ml) and distilled water (8.1 ml). CAN (7.5 g, 13.7 mmol) was added an the mixture was stirred at room temperature for 24 h. The reaction was basified with NH₄OH solution to pH 6-7, poured into brine solution (50 ml) and extracted with EtOAc (3×). The combined organic layers were dried over Na₂SO₄ and concentrated in vacuo. The crude was purified by flash silica gel chromatography (DCM/MeOH, gradient from 100:0 to 90:10) affording 7 (823 mg, 52%).

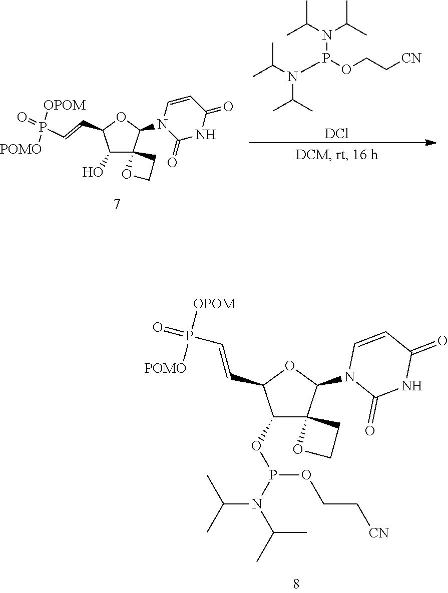

Under a N₂ atmosphere 7 (952 mg, 1.7 mmol) and 4,5-dicyanoimidazole (DCI, 166 mg, 1.4 mmol) were dissolved in dry DCM (9.2 ml). 2-cyanoethyl-N,N,N′,N′-tetraisopropylphosphordiamidit (0.684 ml, 2.2 mmol) was added an the mixture was stirred at room temperature for 16 h. Another portion of 2-cyanoethyl-N,N,N′,N′-tetraisopropylphosphordiamidit (0.158 ml, 0.5 mmol) was added and the mixture was at room temperature for 3 h. The organic layers were poured in water and washed with water (2×) and brine (1×). The organic layer was dried over Na₂SO₄ and concentrated in vacuo. The crude was purified by flash silica gel chromatography (heptane with 0.15% TEA/EtOAc with 0.15% TEA, gradient from 100:0 to 0:100) affording 8 (532 mg, 41%). ¹H NMR (400 MHz, DMSO-d₆) δ ppm 1.12-1.16 (m, 18H), 1.17-1.24 (m, 12H), 2.52-2.78 (m, H), 2.79-2.89 (m, 2H), 3.60-3.79 (m, 3H), 3.79-3.91 (m, 1H), 4.16-4.29 (m, 1H), 4.32-4.56 (m, 3H), 5.56-5.67 (m, 5H), 5.88-5.94 (m, 1H), 6.05-6.21 (m, 1H), 6.67-6.97 (m, 1H), 7.57-7.68 (m, 1H), 11.43-11.50 (m, 1H). ³¹P NMR (162 MHz, DMSO-d₆) δ ppm 16.4, 17.2, 148.7, 150.7 LCMS: Method A, Rt: 1.24, 775.5 [M+H]⁺

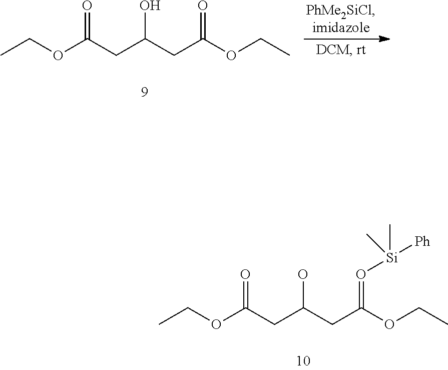

Into a 5 L 3-necked round-bottom flask were added chlorodimethylphenylsilane (183.90 g, 1077.25 mmol) and imidazole (173.35 g, 2546.24 mmol) and DCM (3000 mL) at room temperature. To the above mixture was added 9 (200 g, 979.32 mmol) dropwise over 2 h at 0 °C. The resulting mixture was stirred for additional overnight at room temperature. The reaction was quenched with water at room temperature. The resulting mixture was extracted with CH₂Cl₂ (2×2 L). The combined organic layers were washed with brine (1×2 L), dried over anhydrous Na₂SO₄. After filtration, the filtrate was concentrated under reduced pressure. The residue was purified by flash silica gel chromatography, eluted with PE/EA=10:1 to afford 10 (260 g, 78%).

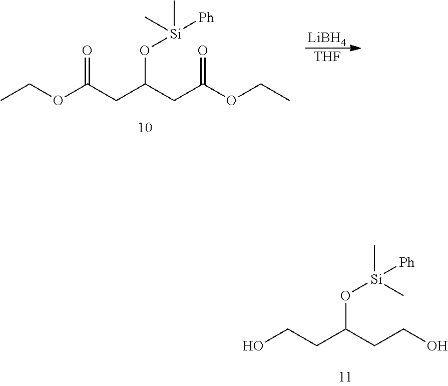

Into a 10 L 4-necked round-bottom flask were added LiBH₄ (50.19 g, 2304.45 mmol) and THF (6500 mL) at room temperature. To the above mixture was added 10 (260 g, 768.15 mmol) dropwise over 1.5 h at 0 °C. The resulting mixture was stirred overnight at room temperature. The reaction was quenched by the addition of sat. NH₄Cl (aq.) (3 L) at 0 °C. The resulting mixture was extracted with CH₂Cl₂ (2×1 L). The combined organic layers were washed with brine (1×2 L), dried over anhydrous Na₂SO₄. After filtration, the filtrate was concentrated under reduced pressure. The residue was purified by flash silica gel chromatography, eluted with PE/EA (1:1) to afford 11 (120 g, 61%).

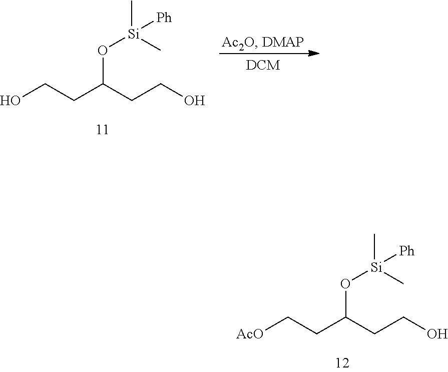 [SMILES: C[Si](C)(OC(CCO)CCO)c1ccccc1 ; CC(=O)OCCC(CCO)O[Si](C)(C)c1ccccc1]

Into a 10 L 4-necked round-bottom flask were added 11 (120 g, 471.69 mmol), DCM (6000 mL), Pyridine (37.68 g, 476.41 mmol) and DMAP (5.76 g, 47.17 mmol) at room temperature. To the above mixture was added Ac₂O (48.64 g, 476.41 mmol) dropwise over 1 h at 0 °C. The resulting mixture was stirred for additional overnight at room temperature. The reaction was quenched with NaHCO₃(aq.) at room temperature. The resulting mixture was extracted with CH₂Cl₂ (2×2 L). The combined organic layers were washed with brine (1×2 L), dried over anhydrous Na₂SO₄. After filtration, the filtrate was concentrated under reduced pressure. The residue was purified by flash silica gel chromatography, eluted with PE/EA (1:1) to afford 12 (64 g, 46%).

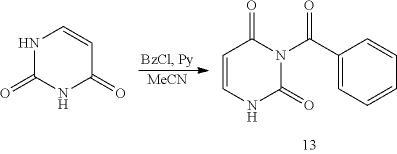

Into a 1 L 3-necked round-bottom flask were added uracil (70 g, 624.50 mmol), acetonitrile (210 mL) and Pyridine (123.50 g, 1.56 mol) at room temperature. To the above mixture was added benzyl chloroformate (234.38 g, 1.37 mol) dropwise over 1.5 h at 0 °C. The resulting mixture was stirred for additional overnight at room temperature. The resulting mixture was extracted with EtOAc (2×500 mL). The combined organic layers were washed with brine (1×500 mL), dried over anhydrous Na₂SO₄. After filtration, the filtrate was concentrated under reduced pressure. The crude product was re-crystallized from MeOH (200 mL) to afford 13 (80 g, 59%).

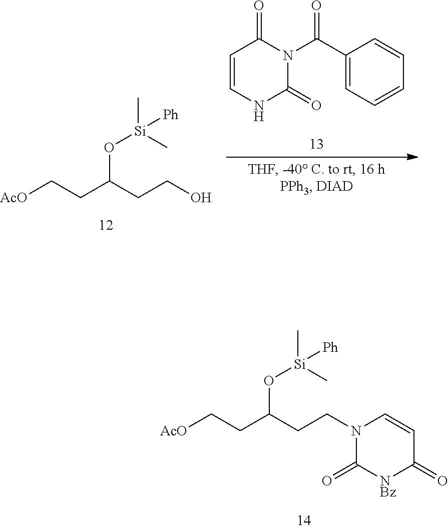

Into a 3 L 3-necked round-bottom flask were added 12 (64 g, 215.89 mmol), PPh₃ (124.58 g, 474.97 mmol) and THF (1600 mL) at room temperature. To the above mixture was added 13 (93.35 g, 431.79 mmol) and DIAD (96.04 g, 474.97 mmol) dropwise over 2 h at −10 °C. The resulting mixture was stirred for additional 3 h at room temperature. The reaction was quenched by the addition of water/ice (1 L) at 0 °C. The resulting mixture was extracted with EtOAc (2×1 L). The combined organic layers were washed with brine (1×1 L), dried over anhydrous Na₂SO₄. After filtration, the filtrate was concentrated under reduced pressure. The residue was purified by flash silica gel chromatography, eluted with PE/EA (1:1) to afford 14 (80 g, 75%).

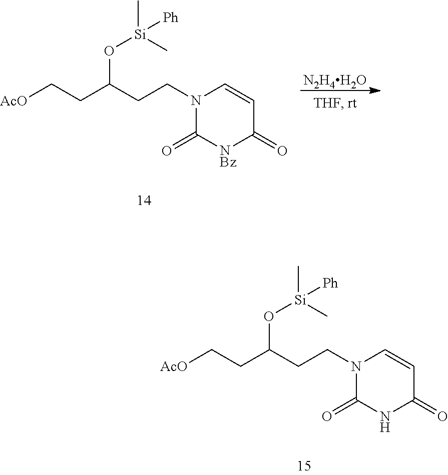

Into a 2 L 3-necked round-bottom flask were added 14 (80 g, 161.74 mmol) and THF (800 mL) at room temperature. To the above mixture was added hydrazine hydrate (12.15 g, 242.61 mmol) dropwise over 30 min at 0 °C. The resulting mixture was stirred for additional 3 h at room temperature. The resulting mixture was concentrated under reduced pressure. The residue was purified by flash silica gel chromatography, eluted with PE/EA (1:1) to afford 15 (55 g, 87%).

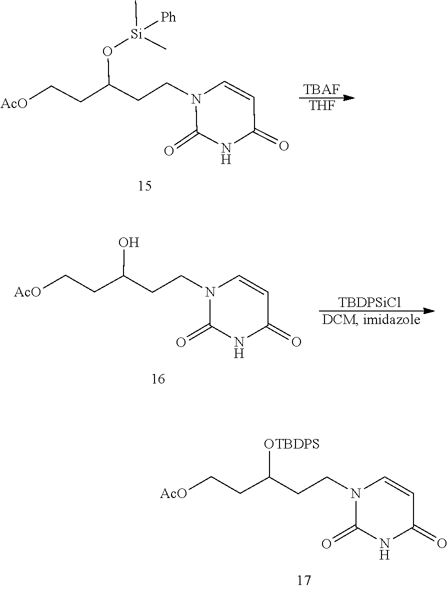

Into a 500 mL 3-necked round-bottom flask were added 15 (55 g, 140.84 mmol) and THF (550 mL) at room temperature. To the above mixture was added TBAF (36.82 g, 140.84 mmol) dropwise over 0.5 h at 0 °C. The resulting mixture was stirred for additional 3 h at room temperature. The resulting mixture was extracted with CH₂Cl₂ (2×1 L). The combined organic layers were washed with brine (1×1 L), dried over anhydrous Na₂SO₄. After filtration, the filtrate was concentrated under reduced pressure. This resulted in 16 (40 g, crude) which was used in the next step without further purification.

Into a 1 L 3-necked round-bottom flask were added 16 (40 g, 156.09 mmol), imidazole (31.42 g, 468.27 mmol) and THF (400 mL) at room temperature. To the above mixture was added tert-butyl(chloro)diphenylsilane (85.81 g, 312.18 mmol) dropwise 1 h at 0 °C. The resulting mixture was stirred for additional 3 h at room temperature. The resulting mixture was extracted with EtOAc (2×400 mL). The combined organic layers were washed with brine (1×400 mL), dried over anhydrous Na₂SO₄. After filtration, the filtrate was concentrated under reduced pressure. The residue was purified by flash silica gel chromatography, eluted with PE/EA (1:1) to afford 17 (52 g, 67%).

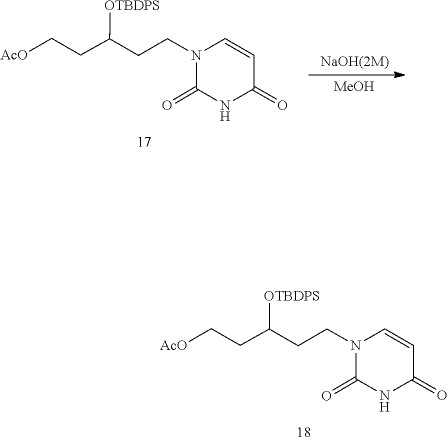

Into a 1 L 3-necked round-bottom flask were added 17 (52 g, 105.12 mmol) and methanol (500 mL) at room temperature. To the above mixture was added NaOH (2 M, 100 mL) dropwise over 1 h at 0 °C. The resulting mixture was stirred for additional 3 h at room temperature. The resulting mixture was extracted with EtOAc (2×200 mL). The combined organic layers were washed with brine (1×200 mL), dried over anhydrous Na₂SO₄. After filtration, the filtrate was concentrated under reduced pressure. The residue was purified by trituration with MeOH (100 mL) and the solids were collected by filtration to afford 18 (40.2 g, 84%).

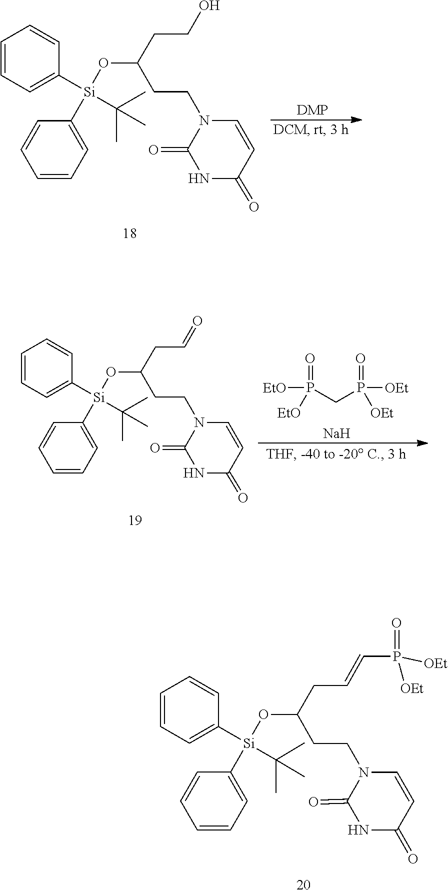

Under a N₂ atmosphere 18 (2.4 g, 5 mmol) was dissolved in DCM (75 ml). DMP (3.0 g, 7 mmol) was added at 0 °C and the mixture was stirred at rt for another for 2 h. To the mixture was added 50 ml of a mixture of 30% aq. Na₂S₂O₃ solution and sat. aq. NaHCO₃ (1:1) and the aqueous layer was extracted with EtOAc (3×). The combined organic layers were dried over Na₂SO₄ and concentrated in vacuo affording 19 as a crude product, which was used in the next step without further purification.

Under a N₂ atmosphere NaH (60% dispersion in mineral oil, 440 mg, 18 mmol) was suspended in THF (10 ml) and the mixture was cooled to −40 °C. Tetraethyl methylenediphosphonate (3.0 ml, 12 mmol) was added and the mixture was stirred at −40° for 30 min. To this mixture a solution of 19 (2.3 g, 5 mmol) in THF (25 ml) was added dropwise over a period of 10 min at −40 °C and the mixture was stirred for 100 min while the mixture was allowed to warm to −10 °C. The crude reaction mixture was poured into water and the product was extracted into ethyl acetate (3×). The organic layers were dried over anhydrous Na₂SO₄ and concentrated in vacuo. The crude was purified by flash silica gel chromatography (heptane/EtOAc:EtOH (3:1), gradient from 100:0 to 0:100) affording 20 as an inseparable mixture with tetraethyl methylene-diphosphonate (2.1 g, 35%).

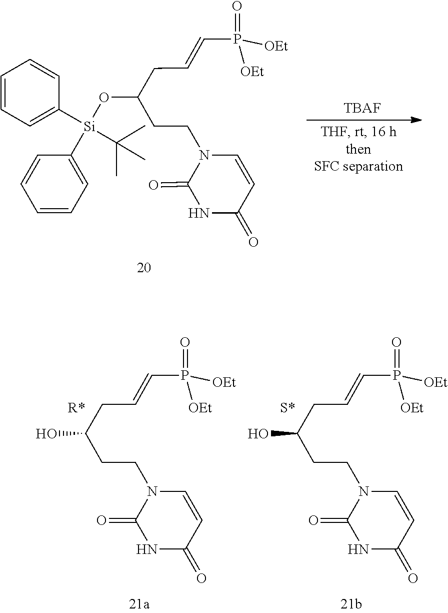 [SMILES: CCOP(=O)(/C=C/CC(CCn1ccc(=O)[nH]c1=O)O[Si](c1ccccc1)(c1ccccc1)C(C)(C)C)OCC ; "TBAF" ; CCOP(=O)(/C=C/C[C@@H](O)CCn1ccc(=O)[nH]c1=O)OCC ; CCOP(=O)(/C=C/C[C@H](O)CCn1ccc(=O)[nH]c1=O)OCC]

Under a N₂ atmosphere 20 (2.1 g, 1.8 mmol) was dissolved in THF (31 ml), TBAF (1M in THF, 2.3 ml, 2.3 mmol) was added at room temperature and the mixture was stirred at 16 h. The solvents were removed in vacuo and the crude was purified by flash silica gel chromatography (DCM/MeOH, gradient from 100:0 to 94:6) affording 21a and 21b (562 mg, 91%) as the racemate. A purification was performed via Prep SFC (Stationary phase: Chiralpak Diacel AD 20×250 mm, Mobile phase: CO₂, EtOH+0.4 iPrNH₂). 21a and 21b were each again purified via Prep SFC (Stationary phase: Torus Diol 30×150 mm, Mobile phase: CO₂, MeOH+0.4 iPrNH₂) affording 21a (192 mg, 31%). 21b was furthermore purified via Prep SFC (Stationary phase: Chiralpak Diacel AD 20×250 mm, Mobile phase: CO₂, EtOH+0.4 iPrNH₂) affording 21b (163 mg, 27%).

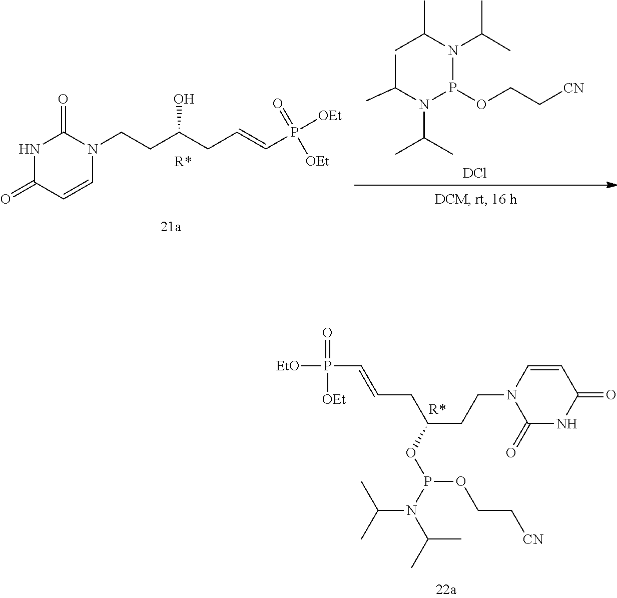

Under a N₂ atmosphere 7 (192 mg, 0.55 mmol) and 4,5-dicyanoimidazole (DCI, 56 mg, 0.47 mmol) were dissolved in dry DCM (3 ml). 2-cyanoethyl-N,N,N′,N′-tetraisopropylphosphordiamidit (0.23 ml, 0.72 mmol) was added an the mixture was stirred at room temperature for 16 h. The mixture was concentrated in vacuo and the crude was purified by flash silica gel chromatography (heptane with 0.15% TEA/EtOAc with 0.15% TEA, gradient from 100:0 to 0:100 then DCM/MeOH, gradient from 100:0 to 95:5) affording 22a (205 mg, 68%). ¹H NMR (400 MHz, acetonitrile-d₃) 6 ppm 1.14-1.22 (m, 12H), 1.23-1.30 (m, 6H), 1.78-1.90 (m, 2H), 2.50-2.65 (m, 2H), 2.65-2.72 (m, 2H), 3.56-3.78 (m, 4H), 3.78-3.94 (m, 2H), 3.94-4.10 (m, 5H), 5.49-5.57 (m, 1H), 5.72-5.87 (m, 1H), 6.59-6.80 (m, 1H), 7.27-7.37 (m, 1H), 8.79-9.00 (m, 1H). ³¹P NMR (162 MHz, acetonitrile-d₃) 6 ppm 16.9, 17.2, 146.8, 147.8. LCMS: Method A, Rt: 1.00, 545.2 [M−H]−, 1.02, 545.2 [M−H]⁻

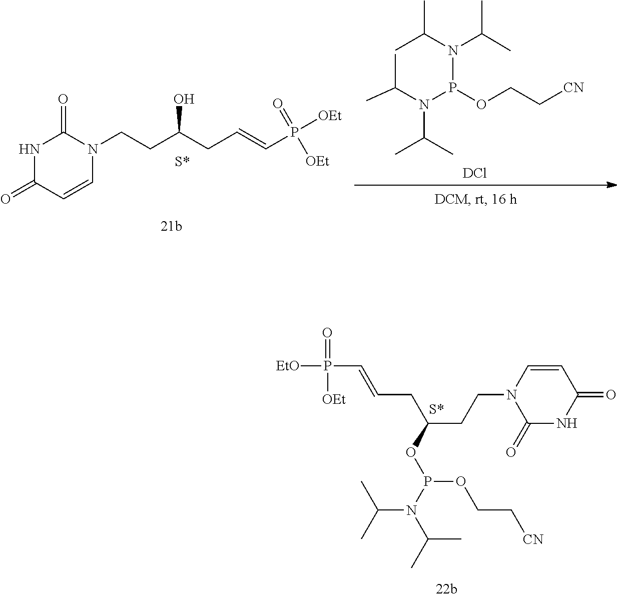

Under a N₂ atmosphere 21b (163 mg, 0.47 mmol) and 4,5-dicyanoimidazole (DCI, 47 mg, 0.40 mmol) were dissolved in dry DCM (2.6 ml). 2-cyanoethyl-N,N,N′,N′-tetraisopropylphosphordiamidit (0.19 ml, 0.61 mmol) was added an the mixture was stirred at room temperature for 16 h. The mixture was concentrated in vacuo and the crude was purified by flash silica gel chromatography (heptane with 0.15% TEA/EtOAc with 0.15% TEA, gradient from 100:0 to 0:100 then DCM/MeOH, gradient from 100:0 to 95:5) affording 22a (196 mg, 76%). ¹H NMR (400 MHz, acetonitrile-d₃) δ ppm 1.16-1.22 (m, 12H), 1.23-1.30 (m, 6H), 1.79-1.91 (m, 2H), 2.48-2.62 (m, 2H), 2.64-2.71 (m, 2H), 3.56-3.76 (m, 4H), 3.79-3.94 (m, 2H), 3.94-4.09 (m, 5H), 5.50-5.58 (m, 1H), 5.71-5.85 (m, 1H), 6.58-6.79 (m, 1H), 7.28-7.39 (m, 1H), 8.81-9.03 (m, 1H). ³¹P NMR (162 MHz, acetonitrile-d₃) δ ppm 16.9, 17.2, 146.8, 147.8. LCMS: Method A, Rt: 0.96, 545.3 [M−H]−, 0.99, 545.3 [M−H]⁻

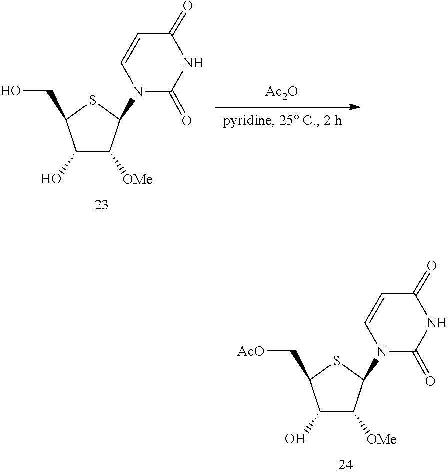 [SMILES: "Ac2O" ; CO[C@@H]1[C@H](O)[C@@H](CO)S[C@H]1n1ccc(=O)[nH]c1=O ; CO[C@@H]1[C@H](O)[C@@H](COC(C)=O)S[C@H]1n1ccc(=O)[nH]c1=O]

To a solution of compound 23 (9.00 g, 32.8 mmol) in pyridine (90.0 mL) was added Ac₂O (3.1 ml, 32.8 mmol) at 10-25 °C. The mixture was stirred at 0 °C for 2 h. The reaction mixture was concentrated under reduced pressure to remove solvent. The residue was purified by flash silica gel chromatography (PE/EtOAc, gradient from 100:0 to 0:100) to afford compound 13 (18.0 g, 86% yield)

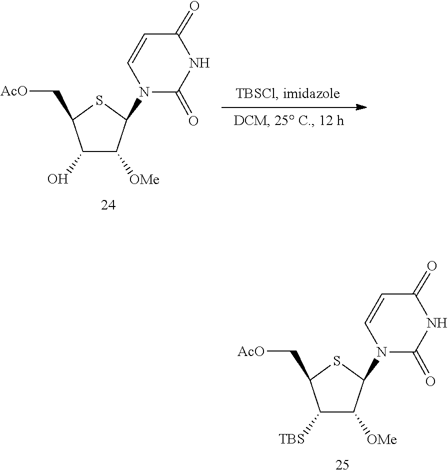

To a solution of compound 24 (18.0 g, 56.9 mmol) in DCM (100 mL) was added imidazole (11.6 g, 171 mmol). Then was slowly added TBSCl (20.9 ml, 170 mmol) in 25.0 mL Dichloromethane at 25 °C. The mixture was stirred at 25 °C for 12 h. The reaction mixture was quenched by addition H₂O 400 mL at 0 °C, then extracted with Ethyl acetate 600 mL (200 mL×3). The combined organic layers were washed with brine 200 mL, dried over Na₂SO₄, filtered, and concentrated under reduced pressure. The crude was purified by flash silica gel chromatography (PE/EtOAc, gradient from 100:0 to 80:20) to afford compound 25 (21.0 g, 85% yield)

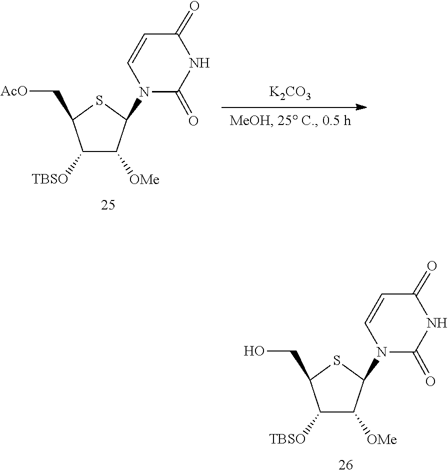

To a solution of compound 25 (21.0 g, 48.7 mmol) in MeOH (150 mL) was added K₂CO₃ (13.5 g, 97.5 mmol). The mixture was stirred at 25 °C for 0.5 h. The reaction mixture was quenched by addition H₂O 350 mL at 0 °C, then extracted with Ethyl acetate 600 mL (200 mL×3). The combined organic layers were washed with brine 200 mL, dried over Na₂SO₄, filtered, and concentrated under reduced pressure. The crude was purified by flash silica gel chromatography (PE/EtOAc, gradient from 100:0 to 0:100) to afford compound 25 (21.0 g, 85% yield). The crude product was further purified by reversed-phase SFC (column: DAICEL CHIRALPAK AS (250 mm×50 mm, 10 um); mobile phase: [Neu-ETOH]; B %: 20%-20%, min) to afford 26 (14.5 g, 84%).

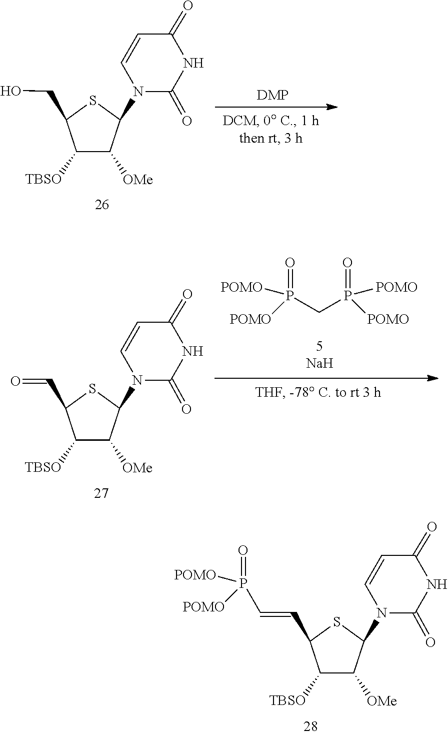

Under a N₂ atmosphere 26 (1.9 g, 5.0 mmol) was dissolved in DCM (50 ml). DMP (3.3 g, 7.8 mmol) was added, and the mixture was stirred at rt for 3 h. To the mixture was added 50 ml of a mixture of 30% aq. Na₂S₂O₃ solution and sat. aq. NaHCO₃ (1:1) and the aqueous layer was extracted with EtOAc (3×). The combined organic layers were dried over Na₂SO₄ and concentrated in vacuo affording 27 (2.5 g) as a crude product, which was used in the next step without further purification.

Under a N₂ atmosphere NaH (60% dispersion in mineral oil, 588 mg, 15 mmol) was suspended in THF (17 ml) and the mixture was cooled to −78 °C. A solution of 5 (7.7 g, 14.5 mmol) in THF (9 ml) was added and the mixture was stirred at −78° for 60 min. To this mixture a solution of 27 (3.0 g, 6 mmol) in THF (17 ml) was added dropwise over a period of 15 min at −78 °C and stirring was continued for 1 h at the same temperature. The mixture was warmed to 0 °C and stirred for 1 h, followed by another 1 h at rt. The crude reaction mixture was poured into 50 ml sat. aq. NH₄Cl solution, and the product was extracted into 50 ml ethyl acetate (3×). The organic layer was washed with brine, dried over anhydrous Na₂SO₄. The solution was then concentrated under reduced pressure, and the residue was purified by flash silica gel chromatography (heptane:EtOAc, gradient from 100:0 to 0:100) to obtain 28 (1.4 g, 40%).

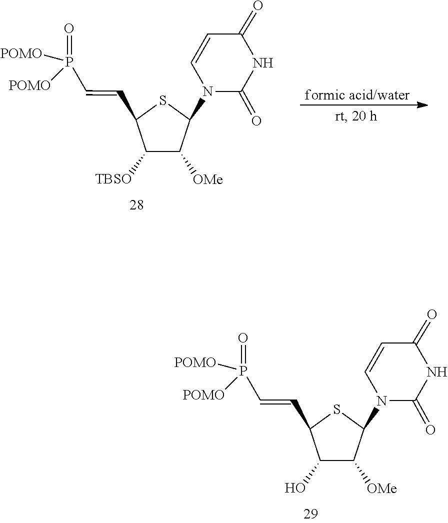

To a mixture of 28 (608 mg, 0.9 mmol) and distilled water (15 ml) was added formic acid (15 ml). The reaction was stirred at room temperature for 20 h. The solvents were removed in vacuo and the crude was purified by flash silica gel chromatography (DCM/MeOH, gradient from 100:0 to 95:5) affording 29 (402 mg, 79%).

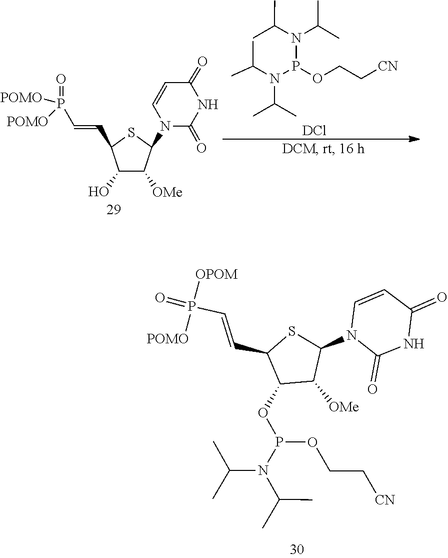

Under a N₂ atmosphere 7 (402 mg, 0.7 mmol) and 4,5-dicyanoimidazole (DCI, 70 mg, 0.6 mmol) were dissolved in dry DCM (3.9 ml). 2-cyanoethyl-N,N,N′,N′-tetraisopropylphosphordiamidit (0.29 ml, 0.9 mmol) was added an the mixture was stirred at room temperature for 16 h. The organic layers were poured in water and washed with water (2×) and brine (1×). The organic layer was dried over Na₂SO₄ and concentrated in vacuo. The crude was purified by flash silica gel chromatography (heptane with 0.15% TEA/EtOAc with 0.15% TEA, gradient from 100:0 to 0:100) affording 30 (163 mg, 30%) and another fraction of 30 (279 mg, 36%, 70% purity). ¹H NMR (400 MHz, acetonitrile-d₃) δ ppm 1.15-1.22 (m, 30H), 2.63-2.71 (m, 2H), 3.43-3.49 (m, 3H), 3.56-3.68 (m, 2H), 3.68-3.79 (m, 1H), 3.79-3.88 (m, 1H), 3.92-4.00 (m, 1H), 4.09-4.22 (m, 1H), 4.29-4.39 (m, 1H), 5.57-5.71 (m, 5H), 5.99-6.15 (m, 2H), 6.79-7.00 (m, 1H), 7.79-7.85 (m, 1H), 8.82-9.07 (m, 1H). ³¹P NMR (162 MHz, acetonitrile-d₃) δ ppm 15.7, 15.9, 149.9, 150.3. LCMS: Method A, Rt: 1.23, 779.4 [M+H]⁺, 1.24, 779.4 [M+H]⁺.

### Example 4—Example of Unlocked Vinyl Phosphonate Synthesis

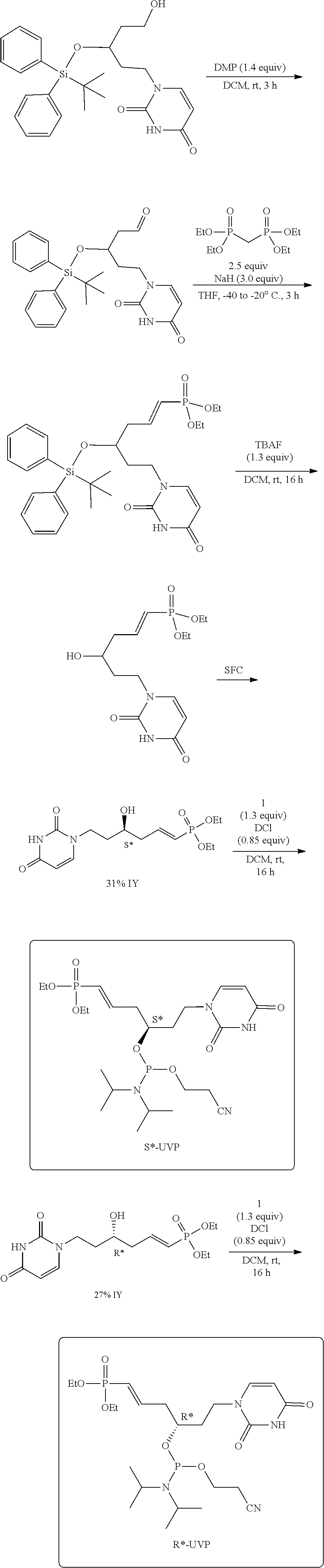

### LCMS Method:

| | | | | | Flow | |
|---|---|---|---|---|---|---|
| | | | | | ------------ | Run |
| Method code | Instrument | column | mobile phase | gradient | Col T | time |
| Method A | Waters: | Waters: | A: 0.1% | From | 0.8 | 2.0 |
| | Acquity ® | BEH | NH₄HCO₃ | 100% A to | ------- | |
| | UPLC ®- | (1.7 μm, | in 95% H2O + 5% | 5% A in | 55 | |
| | DAD and | 2.1*50 mm) | CH₃CN | 1.3 min, | | |
| | SQD | | B: CH₃CN | hold | | |
| | | | | 0.7 min | | |

CPG (25 umol, 30 mg) with synthesized n-1 oligonucleotide with 5′-DMTr on was placed in a 1 mL syringe provided with a filter and cap.

Step 1. The syringe was filled with 3% dichloroacetic acid in DCM and kept for 20 min at ambient temperature. Solvent was removed and CPG was washed 6 times with 0.6 ml of acetonitrile, 3 times with dry DCM, then dried in vacuum for 30 min.

Step 2. The syringe was filled with 200 μL of 0.1 M solution of phosphonate amidite in dry acetonitrile and 200 μL of 0.25 M 5-(thylthio)-1H-tetrazole solution in acetonitrile. The syringe was tightly closed and left gently shaken for 40 min at the ambient temperature. Solvents were removed and the operation repeated second time. Solvents were removed and CPG was washed with 0.6 ml of acetonitrile 6 times. 0.05 M Solution of iodine in pyridine/water 9:1 (v:v) was added and kept for 15 min, repeated twice. CPG was washed 6 times with 0.6 ml of acetonitrile, 3 times with dry DCM, then dried in vacuum for 30 min. An aliquot of CPG (2 mg) was taken, treated with AMA for 10 min at 60 °C, and MW of the obtained oligo was checked by LCMS. If the coupling was not running up to completion the step 2 was repeated.

Step 3. Dry CPG in the syringe was treated with 250 μL of 3.5% solution of trimethylsilyl iodide in acetonitrile/pyridine 50:1 (v/v) for 20 min at ambient temperature. This treatment was repeated 3 times. Solvent was removed and CPG was washed 6 times with 0.6 ml of acetonitrile, then treated with solution of 4% mercaptoethanol in pyridine/triethylamine 1:1 (v:v) for 20 min at ambient temperature. Solvent was removed and CPG was washed 6 times with 0.6 ml of acetonitrile, 3 times with dry DCM, then dried in vacuum for 30 min. An aliquot of CPG (2 mg) was taken, treated with AMA for 10 min at 60 °C, and MW of the obtained oligo was checked by LCMS. If the deprotection was not running up to completion the step 3 was repeated.

Step 4. CPG in the syringe was treated with 200 μL of AMA for 3 h and filtered. Solid support was washed 5 times with 250 μL of water. Filtrate and washings were combined, diluted up to 10 mL and target compound isolated by ion-exchange chromatography. The column GE Source 15Q 250×10 mm. Buffer A was 20 mM sodium phosphate in acetonitrile/water 9:1 (v:v), buffer B was 1.8 M sodium borate in buffer A (pH 7.5). Gradient from 5% to 60% over 10 column volumes was used, target compounds were eluted at 25-35% B. Obtained oligonucleotides were desalted and used for annealing.

Step 5. Annealing was achieved by mixing of equimolar amount of sense and antisense stands. Obtained siRNA were analyzed by LCMS at denaturizing conditions at 80 °C.

### Examples of Synthesized AS

| | | Purity by | SEQ |
|---|---|---|---|
| | | HPLC, | ID |
| AS Oligonucleotide | MS | % | NO |
| VP(Ecc)mUpsfGpsmCfCmUfAmAfUmGfAmGfCmCfAmCfAmCfUmUpsmUpsmU | 6919.5 | 99 | 1 |
| VP(Ecc)mUpsfUpsmGpsfCmGfA(UNA-A)fAmCfAmAfUmUfCmGfAmUfAmUmGpsmA | 7017.6 | 98 | 2 |
| DMP(RS)-mUpsfGpsmCfCmUfAmAfUmGfAmGfCmCfAmCfAmCfUmUpsmUpsmU | 6950.5 | 99 | 3 |
| DMP(SR)-mUpsfGpsmCfCmUfAmAfUmGfAmGfCmCfAmCfAmCfUmUpsmUpsmU | 6950.5 | 99 | 4 |
| DMP(RR)-mUpsfGpsmCfCmUfAmAfUmGfAmGfCmCfAmCfAmCfUmUpsmUpsmU | 6950.5 | 99 | 5 |
| DMP(SS)-mUpsfGpsmCfCmUfAmAfUmGfAmGfCmCfAmCfAmCfUmUpsmUpsmU | 6950.5 | 99 | 6 |
| P(ccB)mUpsfUpsmGpsfCmGfA(UNA-A)fAmCfAmAfUmUfCmGfAmUfAmUmGpsmA | 7047.7 | 99 | 7 |
| P(ccB)mUpsfGpsmCfCmUfAmAfUmGfAmGfCmCfAmCfAmCfUmUpsmUpsmU | 6949.2 | 99 | 8 |
| P(tcB)mUpsfUpsmGpsfCmGfA(UNA-A)fAmCfAmAfUmUfCmGfAmUfAmUmGpsmA | 7047.7 | 98 | 9 |
| P(tcB)mUpsfGpsmCfCmUfAmAfUmGfAmGfCmCfAmCfAmCfUmUpsmUpsmU | 6949.5 | 99 | 10 |

### Examples of Synthesized siRNAs

| | | Purity | SEQ |
|---|---|---|---|
| siRNA | MW | By HPLC | ID NO |
| (invabasic)psmUpsmCmAmUmAmU(J2- | 14680 | 93% | 117 |
| C15AdaC)mGfAfAfUmUmGmUmUmUmCmGmCmApsmAps(invabasic) | | | |
| P(ccB)mUpsfUpsmGpsfCmGfA(UNA-A)fAmCfAmAfUmUfCmGfAmUfAmUmGpsmA | | | |
| (invabasic)psmUpsmCmAmUmAmU(J2- | 14650 | 98% | 112 |
| C15AdaC)mGfAfAfUmUmGmUmUmUmCmGmCmApsmAps(invabasic) | | | |
| VP(Ecc)mUpsfUpsmGpsfCmGfA(UNA-A)fAmCfAmAfUmUfCmGfAmUfAmUmGpsmA | | | |
| (invabasic)psmUpsmCmAmUmAmU(J2- | 14680 | 95% | 119 |
| C15AdaC)mGfAfAfUmUmGmUmUmUmCmGmCmApsmAps(invabasic) | | | |
| P(tcB)mUpsfUpsmGpsfCmGfA(UNA-A)fAmCfAmAfUmUfCmGfAmUfAmUmGpsmA | | | |
| mApsmApsfGmUfGmUfGmGfCmUfCmAfUmUfAmGfGmCfApsmUpsmU-TEG-Chol | 14639 | 98% | 121 |
| VP(Ecc)mUpsfGpsmCfCmUfAmAfUmGfAmGfCmCfAmCfAmCfUmUpsmUpsmU | | | |
| mApsmApsfGmUfGmUfGmGfCmUfCmAfUmUfAmGfGmCfApsmUpsmU-TEG-Chol | 14670 | 98% | 1210 |
| P(tcB)mUpsfGpsmCfCmUfAmAfUmGfAmGfCmCfAmCfAmCfUmUpsmUpsmU | | | |
| mApsmApsfGmUfGmUfGmGfCmUfCmAfUmUfAmGfGmCfApsmUpsmU-TEG-Chol | 14670 | 98% | 128 |
| P(ccB)mUpsfGpsmCfCmUfAmAfUmGfAmGfCmCfAmCfAmCfUmUpsmUpsmU | | | |
| mApsmApsfGmUfGmUfGmGfCmUfCmAfUmUfAmGfGmCfApsmUpsmU-TEG-Chol | 14671 | 92% | 123 |
| DMP(RS)mUpsfGpsmCfCmUfAmAfUmGfAmGfCmCfAmCfAmCfUmUpsmUpsmU | | | |
| mApsmApsfGmUfGmUfGmGfCmUfCmAfUmUfAmGfGmCfApsmUpsmU-TEG-Chol | 14671 | 90% | 124 |
| DMP(SR)mUpsfGpsmCfCmUfAmAfUmGfAmGfCmCfAmCfAmCfUmUpsmUpsmU | | | |
| mApsmApsfGmUfGmUfGmGfCmUfCmAfUmUfAmGfGmCfApsmUpsmU-TEG-Chol | 14671 | 90% | 125 |
| DMP(RR)mUpsfGpsmCfCmUfAmAfUmGfAmGfCmCfAmCfAmCfUmUpsmUpsmU | | | |
| mApsmApsfGmUfGmUfGmGfCmUfCmAfUmUfAmGfGmCfApsmUpsmU-TEG-Chol | 14671 | 90% | 126 |
| DMP(SS)mUpsfGpsmCfCmUfAmAfUmGfAmGfCmCfAmCfAmCfUmUpsmUpsmU | | | |

### HTRA KD by Stable Phosphates in ARPE-19

| | | 0 nM | 0.1 nM | 1 nM | 10 nM |
|---|---|---|---|---|---|
| No oligo | | 101.8 | | | |
| control | | | | | |
| 1 | 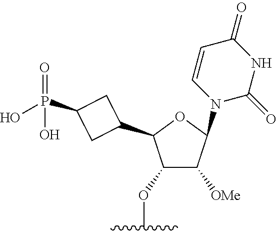 | | 2.40 | 1.14 | 1.67 |
| 2 | 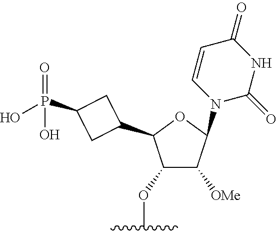 | | 2.66 | 1.78 | 1.41 |
| 3 | 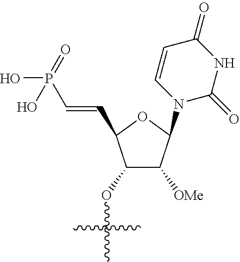 | | 5.53 | 2.71 | 1.88 |
| 4 | 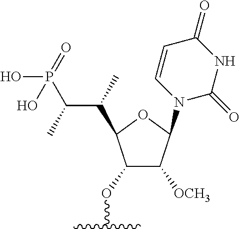 | | 9.28 | 2.17 | 1.83 |
| 5 | 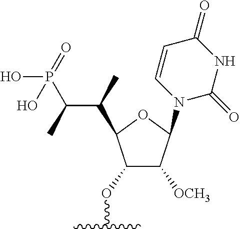 | | 10.19 | 3.93 | 2.51 |
| 6 | 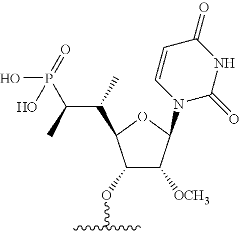 | | 35.43 | 6.79 | 2.35 |
| 7 | 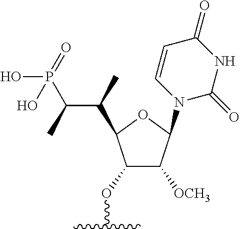 | | 78.10 | 27.31 | 10.16 |

The preceding table shows the percent HTRA remaining (n=3 samples) at 3 days in vitro for varying concentrations of oligonucleotides with the identifies phosphate attached, The oligonucleotides in 1 and 2 were different, and the oligonucleotides in the remaining samples were identical but for the identified phosphate.

### Novel Stable Phosphate Results KD at Week 4 Post-Intravitreal Injection

| | 2 week | 4 week |
|---|---|---|
| | KD (%) | KD (%) |
| PBS | 0 | 0 |
| 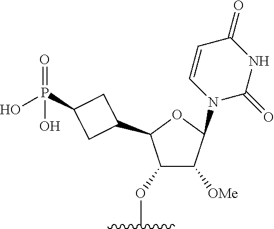 | 61 | 79 |
| 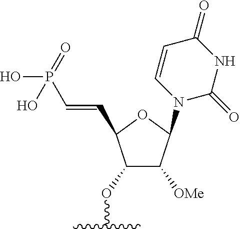 | 48 | 66 |

The preceding table shows novel stable phosphate results KD at week 2 and 4 post-intravitreal injection. The oligonucleotides in the samples were identical but for the identified phosphate.

### Additional Embodiments Include:

1. A compound represented by formula (Ia) or (Ib):

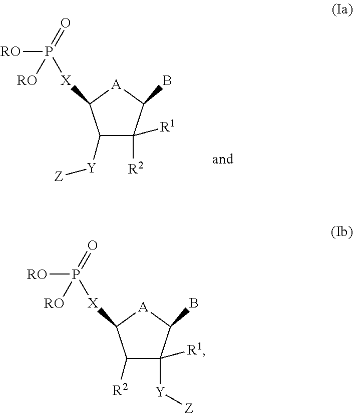

wherein:
X is selected from a 3- to 5-membered cycloalkyl, —CHCH—, a 3- to 5-membered heterocycle, and —CHR³CHR³—;
Y is selected from O or NR′;
R′ is a counterion, H or a protecting group
Z is selected from H, a counterion, an activating group, and an oligonucleotide;
A is selected from O, S, and CR⁴R⁴;
B is a nucleobase;
each R is independently selected from an oligonucleotide, a counterion, H and a protecting group, such as C₁-C₅ alkyl and POM or C₁-C₅ alkyl;
each R¹ and R² is independently selected from H, F, OH, and an optionally substituted O-alkyl, provided that R¹ and R² are not each H;
each R³ is independently selected from a C₁-C₃ alkyl;
each R⁴ is independently selected from H, F and C₁-C₅ alkyl;
provided that when A is O, then X is not —CHCH— and when X is cyclopropyl, then at least one of A or Y is not O.
2. The compound of embodiment 1, wherein the compound of formula (Ia) is represented by the following:

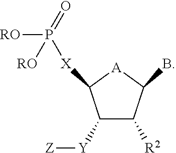

3. The compound of embodiment 1 or 2, wherein X is a cyclobutyl.
4. The compound of embodiment 3, wherein the cyclobutyl is represented by the following:

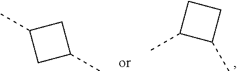

wherein the dashed lines represent connection points to adjacent atoms.
5. The compound of embodiment 1 or 2, wherein X is —CHR³CHR³—, and R³ is a methylene.
6. The compound of embodiment 5, wherein X is selected from the following:

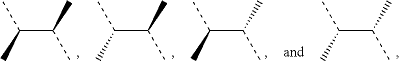

wherein the dashed lines represent connection points to adjacent atoms.
7. The compound of embodiment 1 or 2, wherein X is a 3- to 5-membered heterocycle selected from:

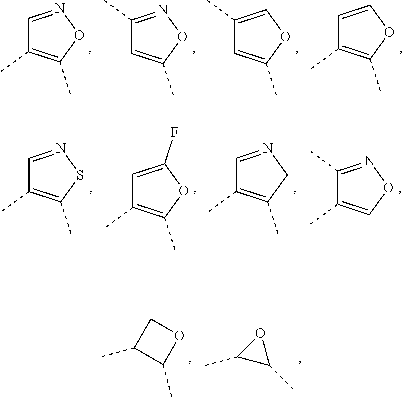

wherein the dashed lines represent connection points to adjacent atoms.
8. The compound of any one of embodiment 1 to 7, wherein Y is O.
9. The compound of any one of embodiment 1 to 8, wherein Z is an activating group.
10. The compound of embodiment 9, wherein the activating group is represented by:

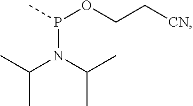

wherein the dashed line represents a connection point to the adjacent atom.
11. The compound of any one of embodiment 1 to 8, wherein Z is an oligonucleotide.
12. The compound of embodiment 11, wherein the oligonucleotide is an antisense strand of RNA, preferably an antisense strand of siRNA.
13. The compound of embodiment 11 or 12, wherein the 5′-end of the oligonucleotide is connected to Y.
14. The compound of any one of embodiment 1 to 13, wherein A is O.
15. The compound of any one of embodiment 1 to 13, wherein A is CH₂ or CHF.
16. The compound of embodiment 15, wherein X is —CHCH—.
17. The compound of any one of embodiment 1 to 16, wherein B is a uracil.
18. The compound of any one of embodiment 1 to 17, wherein R¹ is H and R² is OMe, OEt, MOE, or F.
19. The compound of any one of embodiment 1 to 18, wherein R is a protecting group, such as POM, Et, and Z is an activating group.
20. The compound of any one of embodiment 1 to 18, wherein R is H and Z is an oligonucleotide.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

We claim:

1. A compound represented by formula (Ia) or (Ib): 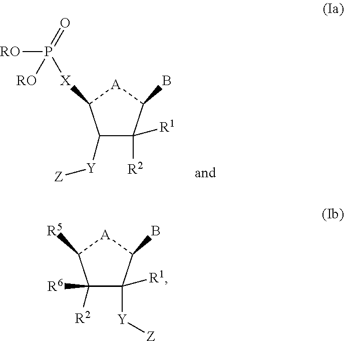
wherein:
X is selected from the group consisting of a 3- to 5-membered cycloalkyl, —CHCH—, a 3- to 5-membered heterocycle, and —CHR³CHR³—;
Y is O or NR′;
R′ is a counterion, H or a protecting group;
Z is selected from the group consisting of H, a counterion, an activating group, and an oligonucleotide;
A and the dashed lines connected are optional, and when present, A is selected from the group consisting of O, S, and CR⁴R⁴;
B is a nucleobase;
each R is independently selected from the group consisting of an oligonucleotide, a counterion, H and a protecting group, such as C₁-C₅ alkyl and POM or C₁-C₅ alkyl;
each R¹ and R² is independently selected from the group consisting of H, F, OH, and an optionally substituted O-alkyl, provided that when X is selected from the group consisting of a 3- to 5-membered cycloalkyl, —CHCH—, a 3- to 5-membered heterocycle, and —CHR³CHR³— then R¹ and R² are not each H, or R¹ and R² form an optionally substituted oxetine;
each R³ is a C₁-C₃ alkyl;
each R⁴ is independently selected from the group consisting of H, F and C₁-C₅ alkyl;
one of R⁵ and R⁶ is XP(O)(OR)₂ and the other is H;
provided that when A is O, then X is not —CHCH— and when X is cyclopropyl, then at least one of A or Y is not 0.

2. The compound of claim 1, wherein the compound is of formula (Ia) and is represented by the following:

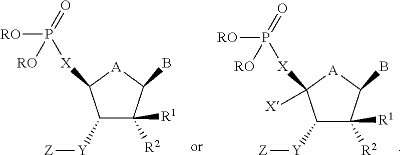

3. The compound of claim 1, wherein X is a cyclobutyl.

4. The compound of claim 3, wherein the cyclobutyl is represented by the following: 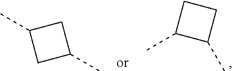
wherein the dashed lines represent connection points to adjacent atoms.

5. The compound of claim 1, wherein X is —CHR³CHR³—, and R³ is a methylene.

6. The compound of claim 5, wherein X is selected from the following: 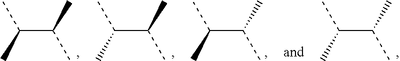
wherein the dashed lines represent connection points to adjacent atoms.

7. The compound of claim 1, wherein X is a 3- to 5-membered heterocycle selected from the group consisting of: 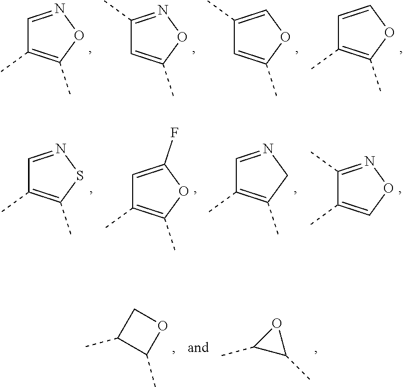
wherein the dashed lines represent connection points to adjacent atoms.

8. The compound of claim 1, wherein the compound of formula (Ia) and (Ib) are represented by the following:

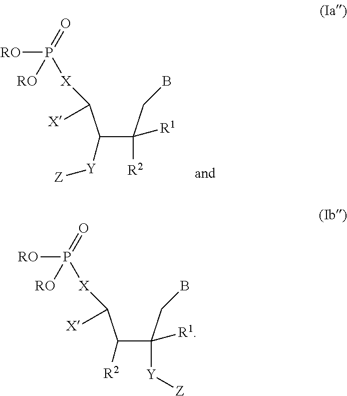

9. The compound of claim 1, wherein R² is an optionally substituted oxetine.

10. The compound of claim 9, wherein X is —CHCH—.

11. The compound of claim 1, wherein Y is O.

12. The compound of claim 1, wherein Z is an activating group.

13. The compound of claim 12, wherein the activating group is represented by: 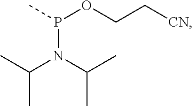
wherein the dashed line represents a connection point to the adjacent atom.

14. The compound of claim 1, wherein Z is an oligonucleotide.

15. The compound of claim 14, wherein the oligonucleotide is an antisense strand of RNA, preferably an antisense strand of siRNA.

16. The compound of claim 14, wherein the 5′-end of the oligonucleotide is connected to Y.

17. The compound of claim 1, wherein A is O.

18. The compound of claim 1, wherein A is CH₂ or CHF.

19. The compound of claim 18, wherein X is —CHCH—.

20. The compound of claim 1, wherein B is a uracil or thymine.

21. The compound of claim 1, wherein R¹ is H and R² is OMe, OEt, MOE, or F.

22. The compound of claim 1, wherein R is a protecting group, such as POM, Et, and Z is an activating group.

23. The compound of claim 1, wherein R is H and Z is an oligonucleotide.

24. The compound of claim 1, wherein the compound is selected from the group consisting of: 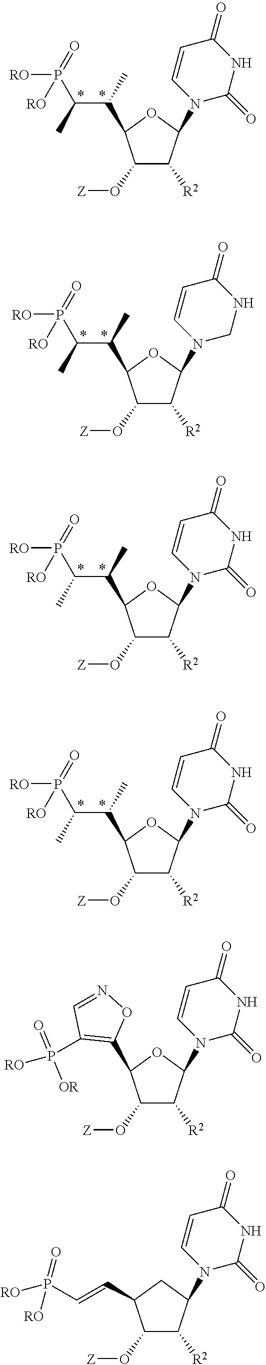 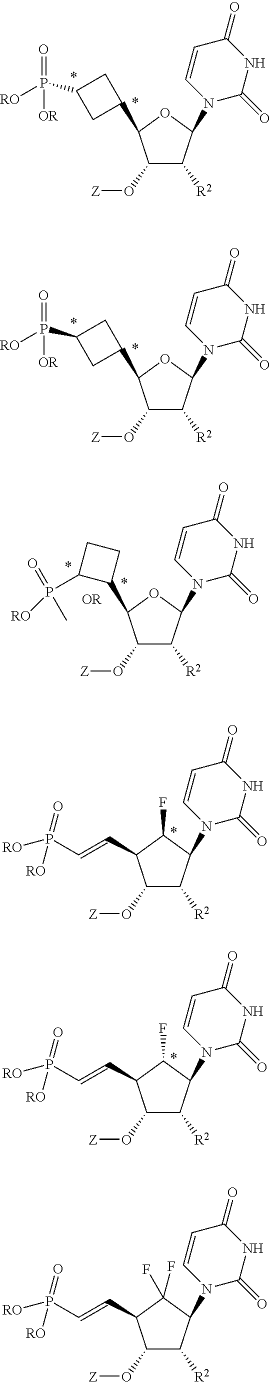 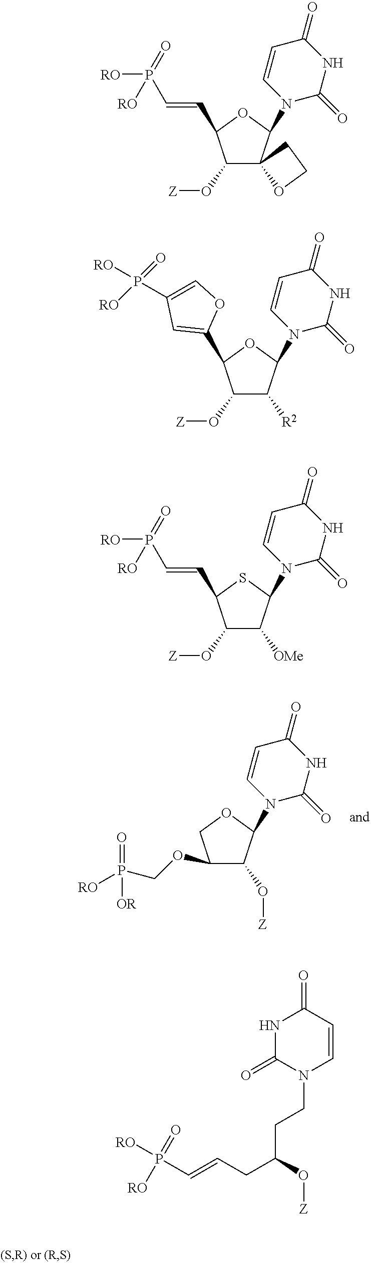
or wherein the compound is a compound of the table wherein the nucleobase is thymine or cytosine.

25. The compound of claim 24, wherein R² is —OMe.

26. The compound of claim 24, wherein R is Et and Z is an activating group.

27. The compound of claim 24, wherein R is H or counterion and Z is an oligonucleotide.
